(19) United States
(12) Patent Application Publication
(10) Pub. No.: US 2024/0218254 A1
(43) Pub. Date: Jul. 4, 2024

(54) ADDITIVE FOR SURFACE ACTIVATION, AND EMULSION COMPOSITION USING SAME

(71) Applicant: MIYOSHI OIL & FAT CO., LTD., Tokyo (JP)

(72) Inventors: Akira YASHITA, Tokyo (JP); Kotaro KANEKO, Tokyo (JP); Koji KAWAI, Tokyo (JP)

(73) Assignee: MIYOSHI OIL & FAT CO., LTD., Tokyo (JP)

(21) Appl. No.: 18/286,274

(22) PCT Filed: Apr. 22, 2022
(86) PCT No.: PCT/JP2022/018555

§ 371 Date: Oct. 10, 2023

(30) Foreign Application Priority Data
Apr. 23, 2021 (JP) 2021-073003

(51) Int. Cl. C09K 23/38; A61K 8/06; A61K 8/36; A61K 8/41; A61Q 19/00; C07C 53/00; C07C 57/03; C07C 215/06; C07C 215/12; C09K 23/16; C09K 23/18; C10M 133/08; C10M 133/36; C10M 173/00; C10N 50/00

(52) U.S. Cl. CPC C09K 23/38; A61K 8/06; A61K 8/36; A61K 8/41; A61Q 19/00; C07C 53/00; C07C 57/03; C07C 215/06; C07C 215/12; C09K 23/16; C09K 23/18; C10M 133/08; C10M 133/36; C10M 173/00; A61K 2800/10; C10M 2201/02; C10M 2215/26; C10N 2050/01

(57) ABSTRACT

The present invention addresses the problem of providing a novel additive for surface activation and an emulsion composition using same, in which an excellent emulsion stability is obtained. The present invention also addresses the problem of improving the usage feel in a cosmetic and the lubrication performance in a lubricant. This additive for surface activation is one in which the following components (A) and (B) are compounded. (A) An amine or ammonium compound having a hydroxyaliphatic hydrocarbon group that contains one or more hydroxy groups and optionally contains an oxygen atom unassociated with the hydroxy groups; and (B) an unsaturated or branched aliphatic acid having 8 to 22 carbon atoms or a salt thereof. In addition, this emulsion composition is one in which the above additive has been added.

### TECHNICAL FIELD

The present invention relates to an additive for surface activation, and an emulsion composition using the same.

### BACKGROUND ART

Emulsion compositions are used in various fields, among which emulsion compositions for use in, for example, cosmetics or lubricants require emulsion stability. A surfactant is typically used in emulsion composition, and the HLB (Hydrophilic-Lipophilic Balance) value of the surfactant provides an indication of emulsification, and a surfactant is selected based on the ratio of oil and water. Surfactants that have been conventionally used include a non-ionic surfactant which is based on, for example, Span or Tween but such non-ionic surfactants have not been particularly favorable as cosmetics or topical products for the skin in terms of the feeling of use such as stickiness. Further, a surfactant that is more excellent in terms of lubricity and is widely applicable for various water-to-oil ratios is in need.

The applicant has proposed an organic ammonium salt (ionic liquid) having a hydrogen-bonding functional group(s) in a cation and/or an anion (Patent documents 1 to 3). It has been found that this organic acid has hydrophilic property, is liquid at room temperature and is excellent in water retaining and moisturizing capabilities.

### PRIOR ART DOCUMENTS

### Patent Documents

[Patent document 1] JP-A-2014-131974
[Patent document 2] JP-A-2014-131975
[Patent document 3] JP-A-2019-023185

### SUMMARY OF THE INVENTION

### Problems to be Solved by the Invention

Nevertheless, no study has ever been focused on the improvement in surfactant capability or in emulsion stability to be brought thereby, and particularly no study has ever been made for a combination of an aliphatic acid and an amine which are respectively an anion and a cation of an organic ammonium salt that is suitable for the present objective.

The present invention has been made in view of the above circumstances, and it is an object of the invention to provide a novel additive for surface activation, and an emulsion composition using the same, and further to improve the feeling of use in cosmetics and the lubricating capability of lubricants.

### Means to Solve the Problems

In order to solve the aforementioned problem, the additive for surface activation according to the present invention is characterized in that the additive is a formulation comprising the following components (A) and (B), wherein the component (A) is an amine or ammonium compound comprising a hydroxyaliphatic hydrocarbon group having one or more hydroxy groups and optionally containing an oxygen atom unassociated with the hydroxy groups, and the component (B) is an unsaturated or branched aliphatic acid having 8 to 22 carbon atoms or a salt thereof.

It is preferred that the additive of the present invention comprise an organic salt formed by: a cation originated from the component (A); and an anion originated from an anionic residue of the component (B), wherein said cation optionally contains a cationic residue of the component (B). The emulsion composition of the present invention is made by adding the additive. The cosmetic of the present invention is made by adding the additive. The lubricant of the present invention is made by adding the additive.

### Effects of the Invention

The emulsion composition to which the additive for surface activation according to the present invention has been added exhibits superior emulsion stability.

The emulsion composition of the present invention is excellent in emulsion stability, has a favorable feeling of use when applied to the skin when the composition is used as a cosmetic, and is excellent in lubricity when the composition is used as a lubricant.

### MODE FOR CARRYING OUT THE INVENTION

The present invention will be described in greater detail hereinbelow.

### (Additive for Surface Activation)

The additive according to the present invention is mainly directed to an additive to be mixed in any process(es) of manufacturing an intended composition, particularly an emulsified composition. The additive as used herein refers to the one having components (A) and (B) being mixed therein. The expression being that the components (A) and (B) are “mixed” as used herein encompasses a case in which the components (A) and (B) have been mixed before finally preparing the additive. The expression also encompasses a case in which a salt is formed by the components (A) and (B) serving as starting materials, and the salt is used as an additive, and a case in which the aforementioned salt is mixed, as needed, with further components such as water to make an additive. The additive as used herein may be a mixture composed only of the components (A) and (B) (including a case of the salts thereof), or a compound further containing a component such as water other than the components (A) and (B) and their salts.

The expression “for surface activation” as used herein in connection with the additive encompasses a meaning that the additive has a functionality as a surfactant.

As for the additive of the present invention, skeletons of the amine compounds in component (A) are not particularly limited, and examples of which include amines (ammonia, primary amine, secondary amine, tertiary amine) such as, although not limited to the following, cyclic amines such as imidazole, pyridine, pyrrolidine, piperidine, pyrroline, pyrazine, triazole, isoquinoline, oxazoline, thiazoline, morpholine, guanidine, pyrimidine, piperazine, triazine, quinoline, indole, quinoxaline, and isoxazoline; and various amino acids (such as glycine, L-alanine, L-valine, L-leucine, L-isoleucine, sarcosine, L-serine, L-threonine, L-cysteine, L-methionine, L-cystathionine, L-asparagine, L-glutamine, citrulline, L-proline, L-hydroxyproline, L-phenylalanine, L-tyrosine, L-tryptophan, 1-methylhistidine, 3-methylhistidine, anserine, carnosine, β-alanine, β-aminoisobutyric acid, γ-aminobutyric acid, ε-aminocaproic acid, glutamic acid, aspartic acid, α-aminoadipic acid, arginine, lysine, histidine, 5-hydroxylysine, and omitin), among which preferred are amines, imidazole, pyridine, pyrrolidine, piperidine and morpholine, and amines are more preferred. Further, they may contain, for example, a substituent group as outlined in the section of [Substituent Group] to be described below.

Examples of the ammonium compound in component (A) include salts of the aforementioned amine compounds, or compounds obtained by quaternizing the aforementioned amine compounds.

The component (A) contains a hydroxyaliphatic hydrocarbon group having one or more hydroxy groups and optionally containing an oxygen atom unassociated with the hydroxy groups.

This hydroxyaliphatic hydrocarbon group may be linear or branched but it is preferred that the group be a branched hydroxyaliphatic hydrocarbon group, and the hydroxyaliphatic hydrocarbon group may be saturated or unsaturated but it is more preferred that the group be a saturated aliphatic hydrocarbon group. Examples of the aliphatic hydrocarbon group include those listed in the section of “Hydrocarbon group” as hereinafter defined.

The linear hydroxyaliphatic hydrocarbon group has at least one hydroxy group, and the hydrocarbon moiety thereof is a linear moiety having, preferably 1 to 22 carbon atoms, more preferably 1 to 18 carbon atoms, even more preferably 1 to 12 carbon atoms, particularly more preferably 1 to 6 carbon atoms; such hydrocarbon moiety may also contain an oxygen atom(s). It is preferred that the number of hydroxy groups in one linear hydroxyaliphatic hydrocarbon group be 1 to 8, more preferably 1 to 6, even more preferably 1 to 3.

The branched hydroxyaliphatic hydrocarbon group may have one or more hydroxy groups, and the hydrocarbon moiety thereof is branched and has preferably 3 to 22, more preferably 3 to 12, even more preferably 3 to 8, particularly preferably 3 to 6, most preferably 4 carbon atoms, and the hydrocarbon moiety optionally contains an oxygen atom(s).

It is preferred that the number of hydroxy groups in one branched hydroxyaliphatic hydrocarbon group be 1 to 8, more preferably 1 to 3, and even more preferably 3. It is preferred that the number of the branched hydroxyaliphatic hydrocarbon groups in the amine or ammonium compound be 1 to 4, more preferably 1 to 3, and even more preferably 1 to 2, and particularly preferably 1.

Here, when the hydrocarbon moiety contains an oxygen atom(s), such oxygen atom(s), for example, form an ether bond, a carbonyl group, an ester bond, an amide bond, a urea bond or a urethane bond in the hydrocarbon moiety. Therefore, in this invention, the expression “the hydrocarbon moiety contains an oxygen atom(s)” encompasses a case where the hydrocarbon moiety is interrupted by, or the hydrogen atom(s) are substituted by a group serving as an oxygen atom-containing atom group that even may contain a hetero atom(s) such as a nitrogen atom or a case where the group is present therein at its base end.

Examples of the linear hydroxyaliphatic hydrocarbon group include, but are not particularly limited to, a group having one hydroxy group such as a hydroxymethyl group, 1-hydroxyethyl group, 2-hydroxyethyl group, 1-hydroxypropan-1-yl group, 2-hydroxypropan-1-yl group, 3-hydroxypropan-1-yl group, 1-hydroxybutan-1-yl group, 2-hydroxybutan-1-yl group, 3-hydroxybutan-1-yl group, 4-hydroxybutan-1-yl group, 5-hydroxypentan-1-yl group, 6-hydroxyhexan-1-yl group, 7-hydroxyheptan-1-yl group, 8-hydroxyoctan-1-yl group, 9-hydroxynonan-1-yl group, 10-hydroxydecane-1-yl group; and a group having two or more hydroxy groups such as a dihydroxyethyl group such as 1,2-dihydroxyethyl group; a dihydroxypropan-1-yl group such as 1,2-dihydroxypropan-1-yl group and 2,3-dihydroxypropan-1-yl group; a trihydroxypropan-1-yl group; a dihydroxybutan-1-yl group such as 1,2-dihydroxybutan-1-yl group, 1,3-dihydroxybutan-1-yl group, 1,4-dihydroxybutan-1-yl group, 2,3-dihydroxybutan-1-yl group, 2,4-dihydroxybutan-1-yl group, and 3,4-dihydroxybutan-1-yl group; a trihydroxybutan-1-yl group such as 1,2,3 trihydroxybutan-1-yl group, 1,2,4 trihydroxybutan-1-yl group, 1,3,4 trihydroxybutan-1-yl group and 2,3,4 trihydroxybutan-1-yl group; a tetrahydroxybutan-1-yl group; a di-, tri-, tetra- or pentahydroxypentan-1-yl group; a di-, tri-, tetra-, penta-, or hexahydroxyhexan-1-yl group; a di-, tri-, tetra-, penta-, hexa- or heptahydroxyheptan-1-yl group; and a di-, tri-, tetra-, penta-, hexa-, hepta-, or octahydroxyoctan-1-yl group. In terms of safety, a 1-hydroxyethyl group and a 2-hydroxypropyl group listed in Japanese standards of quasi-drug ingredients (JSQI) are preferred.

Examples of the branched hydroxyaliphatic hydrocarbon group include, but are not particularly limited to, a group represented by the following formula (IV):

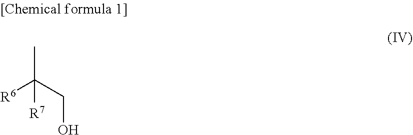

In the formula, R⁶ represents a hydrogen atom, a linear alkyl group having 1 to 8 carbon atoms or a linear hydroxyalkyl group having 1 to 8 carbon atoms. R⁷ represents a linear alkyl group having 1 to 4 carbon atoms or a linear hydroxyalkyl group having 1 to 8 carbon atoms.) Particularly, it is preferred that at least one of the R⁶ and R⁷ in the component (A) be a hydroxyalkyl group having 1 to 4 carbon atoms, and it is more prefeed that both of the R⁶ and R⁷ in the component (A) be a hydroxyalkyl group having 1 to 4 carbon atoms.

In the above formula (IV), it is preferred that the branched hydroxyaliphatic hydrocarbon group have 3 to 6, more preferably 3 to 4 carbon atoms. It is preferred that the number of hydroxyl groups in one branched hydroxyaliphatic hydrocarbon group be 1 to 8, more preferably 1 to 4, even more preferably 2 to 4, particularly preferably 2 to 3, and most preferably 3.

Examples of the hydroxyaliphatic hydrocarbon group as represented in the formula (IV) include, but are not particularly limited to, a 1,3-dihydroxy-2-hydroxymethylpropan-2-yl group, 1,3-dihydroxypropan-2-yl group, 1,3-dihydroxy-2-ethylpropan-2-yl group, 1,3-dihydroxy-2-methylpropan-2-yl group, 1-hydroxy-2-methylpropan-2-yl group, 1-hydroxy-3-methylbutan-2-yl group, 2-hydroxy-2-methylpropan-2-yl group, 2-hydroxy-2-methylbutan-2-yl group, 2-ethyl-2-hydroxybutan-2-yl group, 2-hydroxy-3-methylpentan-2-yl group, 3-ethyl-2-hydroxypentan-2-yl group, 2-ethyl-1-hydroxy-3-methylbutan-2-yl group, 1-hydroxy-3-methyl-2-(1-methylethyl) butan-2-yl group, 2-ethyl-1-hydroxypentan-2-yl group, 1-hydroxy-2-propylpentan-2-yl group, 4-ethyl-3-hydroxyhexan-4-yl group, 3-ethyl-2-hydroxy-2-methylpentan-3-yl group, 2-ethyl-1-hydroxyhexan-2-yl group, 1-hydroxy-2-propylhexan-2-yl group, 2-ethyl-1-hydroxyheptan-2-yl group, 2-ethyl-1-hydroxy-4-methylpentan-2-yl group, 1-hydroxy-2-(1-methylethyl)pentan-2-yl group, 3-ethyl-4-hydroxyheptan-3-yl group, 1,1-dihydroxybutan-2-yl group, 1,1-dihydroxypentan-2-yl group, 1,1-dihydroxy-5-methylhexan-2-yl group, 1,1-dihydroxypropan-2-yl group and 1,1-dihydroxy-4-(-4-hydroxyphenyl)butan-2-yl group, among which, in terms of safety, preferred are, although not limited to the following, a 1,3-dihydroxy-2-hydroxymethylpropan-2-yl group, 1,3-dihydroxy-2-methylpropan-2-yl group, 1-hydroxy-2-methylpropan-2-yl group, and 1,3-dihydroxy-2-methylpropan-2-yl group that are listed in Japanese standards of quasi-drug ingredients (JSQI), Japanese standards of quasi-drug additives, Japanese pharmacopoeia (JP), Japanese pharmaceutical codex (JPC), Japanese pharmaceutical excipients (JPE) and Japan's specifications and standards for food additives (JSFA). Meanwhile, in terms of the feeling of use as cosmetics and lubricity functionality of the additive, preferred are a 1,3-dihydroxy-2-hydroxymethylpropan-2-yl group, 1,3-dihydroxypropan-2-yl group, 1,3-dihydroxy-2-ethylpropan-2-yl group, 1,3-dihydroxy-2-methylpropan-2-yl group and 1-hydroxy-2-methylpropan-2-yl group; and in terms of emulsification capability, more preferred are a 1,3-dihydroxy-2-hydroxymethylpropan-2-yl group, 1,3-dihydroxypropan-2-yl group and 1,3-dihydroxy-2-ethylpropan-2-yl group, among which 1,3-dihydroxy-2-hydroxymethylpropan-2-yl group is even more preferred.

Examples of the substitutes in the component (A) or in a cation originated from the component (A) include not only the hydroxyaliphatic hydrocarbon groups but also organic groups.

### [Organic Group]

The term “organic group” as used herein refers to a group that essentially contains carbon atom(s) and optionally contains at least one further species selected from hydrogen atom, oxygen atom, nitrogen atom, sulfur atom, phosphorus atom, and halogen atom. Examples of atom group(s) contained in the organic group include, but are not limited to, a hydrocarbon group, a heterocyclic group, and a substituent group as set forth in the section of “substituent group” to be hereinafter described. Examples of such include a group in which the substituent group, as set forth in the section of “substituent group” interrupts a hydrocarbon moiety, or the substituent is present at a base end(s) thereof, or a hydrogen atom(s) is/are substituted by the substituent group. The number of carbon atoms in the organic group may be, although not limited to the following, for example, 1 to 22, 1 to 18, 1 to 12, 1 to 8, or 1 to 6.

### [Hydrocarbon Group]

Examples of the hydrocarbon group as used herein include, but are not limited to, a saturated or unsaturated aliphatic hydrocarbon group, a saturated or unsaturated alicyclic hydrocarbon group, an aromatic hydrocarbon group and a hydrocarbon group of any combination thereof. The group may be monovalent or multivalent depending on the context of the application, and examples of a saturated or unsaturated monovalent aliphatic hydrocarbon group include, but are not limited to, a linear or branched alkyl group, alkenyl group, and alkynyl group. Examples of the alkyl group include linear or branched ones, and include, but are not particularly limited to, a methyl group, ethane-1-yl group, propane-1-yl group, 1-methyl ethane-1-yl group, butane-1-yl group, butane-2-yl group, 2-methyl propane-1-yl group, 2-methyl propane-2-yl group, pentane-1-yl group, pentane-2-yl group, hexane-1-yl group, heptane-1-yl group, octane-1-yl group, 2-ethyl hexane-1-yl group, 1,1,3,3-tetramethyl butane-1-yl group, nonane-1-yl group, decane-1-yl group, undecane-1-yl group, dodecane-1-yl group, tridecane-1-yl group, tetradecane-1-yl group, pentadecane-1-yl group, hexadecane-1-yl group, 2-hexyl decane-1-yl group, heptadecane-1-yl group, octadecane-1-yl group, nonadecane-1-yl group, icosane-1-yl group, henicosane-1-yl group, docosane-1-yl group, and 4,8,12-trimethyl tridecane-1-yl group. Examples of the alkenyl group include linear or branched ones, and include, but are not particularly limited to, a vinyl group, prop-1-en-1-yl group, allyl group, isopropenyl group, but-1-en-1-yl group, but-2-en-1-yl group, but-3-en-1-yl group, 2-methylprop-2-en-1-yl group, 1-methylprop-2-en-1-yl group, pent-1-en-1-yl group, pent-2-en-1-yl group, pent-3-en-1-yl group, hent-4-en-1-yl group, 3-methylbut-2-en-1-yl group, 3-methylbut-3-en-1-yl group, hex-1-en-1-yl group, hex-2-en-1-yl group, hex-3-en-1-yl group, hex-4-en-1-yl group, hex-5-en-1-yl group, 4-methylpent-3-en-1-yl group, 4-methylpent-3-en-1-yl group, hept-1-en-1-yl group, hept-6-en-1-yl group, oct-1-en-1-yl group, oct-7-en-1-yl group, non-1-en-1-yl group, non-8-en-1-yl group, dec-1-en-1-yl group, dec-9-en-1-yl group, undec-1-en-1-yl group, undec-10-en-1-yl group, dodec-1-en-1-yl group, dodec-11-en-1-yl group, tridec-1-en-1-yl group, tridec-12-en-1-yl group, tetradec-1-en-1-yl group, tetradec-13-en-1-yl group, pentadec-1-en-1-yl group, pentadec-14-en-1-yl group, hexadec-1-en-1-yl group, hexadec-15-en-1-yl group, heptadec-1-en-1-yl group, heptadec-16-en-1-yl group, octadec-1-en-1-yl group, octadec-9-en-1-yl group, octadec-17-en-1-yl group, nonadec-1-en-1-yl group, icos-1-en-1-yl group, henicos-1-en-1-yl group, and a docos-1-en-1-yl group. Examples of the alkynyl group include linear or branched ones, and include, but are not particularly limited to, an ethynyl, prop-1-yn-1-yl group, prop-2-yn-1-yl group, but-1-yn-1-yl group, but-3-yn-1-yl group, 1-methylprop-2-yn-1-yl group, pent-1-yn-1-yl group, pent-4-yn-1-yl group, hex-1-yn-1-yl group, hex-5-yn-1-yl group, hept-1-yn-1-yl group, hept-6-yn-1-yl group, oct-1-yn-1-yl group, oct-7-yn-1-yl group, non-1-yn-1-yl group, non-8-yn-1-yl group, dec-1-yn-1-yl group, dec-9-yn-1-yl group, undec-1-yn-1-yl group, undec-10-yn-1-yl group, dodec-1-yn-1-yl group, dodec-11-yn-1-yl group, tridec-1-yn-1-yl group, tridec-12-yn-1-yl group, tetradec-1-yn-1-yl group, tetradec-13-yn-1-yl group, pentadec-1-yn-1-yl group, pentadec-14-yn-1-yl group, hexadec-1-yn-1-yl group, hexadec-15-yn-1-yl group, heptadec-1-yn-1-yl group, heptadec-16-yn-1-yl group, octadec-1-yn-1-yl group, octadec-17-yn-1-yl group, nonadec-1-yn-1-yl group, icos-1-yn-1-yl group, henicos-1-yn-1-yl group, and a docos-1-yn-1-yl group.

The saturated or unsaturated alicyclic hydrocarbon group is preferably a saturated alicyclic hydrocarbon group, and examples of which include, but are not particularly limited to, a cyclopropyl group, cyclobutyl group, cyclopentyl group, cyclohexyl group, cycloheptyl group and a cyclooctyl group as monovalent groups, as well as groups containing alicyclic residues of those or other residues.

Examples of the aromatic hydrocarbon group include, but are not particularly limited to, a phenyl group, naphthalene group, and anthracene group, as well as groups containing aromatic ring residues of those or other residues. The group may form a condensed ring together with the substituent groups as set forth in [Substituent Group] to be described below. Examples of the monovalent aromatic hydrocarbon group include, but are not particularly limited to, a phenyl group, 2-methylphenyl group, 3-methylphenyl group, 4-methylphenyl group, 2,4-dimethylphenyl group, 2,5-dimethylphenyl group, 3,4-dimethylphenyl group, 3,5-dimethylphenyl group, 2,4,5-trimethylphenyl group, 2,4,6-trimethylphenyl group, 4-ethylphenyl group, 4-propylphenyl group, 4-isopropylphenyl group, 4-butylphenyl group, 4-tert-butylphenyl group, 4-pentylphenyl group, 4-tert-pentylphenyl group, 2,4-bis(4-tert-pentyl)phenyl group, 1,1,3,3-tetramethylbutylphenyl group, 2-methyl-5-tert-butylphenyl group, 4-pentylphenyl group, 4-hexylphenyl group, 4-heptylphenyl group, 4-octylphenyl group, 4-nonylphenyl group, 4-decanylphenyl group, 4-undecylphenyl group, 4-dodecylphenyl group, 4-tridecylphenyl group, 4-tetradecylphenyl group, 4-pentadecylphenyl group, 4-hexadecylphenyl group, 4-heptadecylphenyl group, 4-octadecylphenyl group, benzyl group, α, α-dimethyl benzyl group, 4-biphenyl group, 2-methoxyphenyl group, 3-methoxyphenyl group, 4-methoxyphenyl group, 2-ethoxyphenyl group, 3-ethoxyphenyl group, 4-ethoxyphenyl group, 2-chlorophenyl group, 2-fluorophenyl group, 4-fluorophenyl group, 2-trifluoromethylphenyl group, 4-trifluoromethylphenyl group, 4-hydroxyphenyl group, 1-naphthyl group, 2-naphthyl group, 1-anthracenyl group, 2-anthracenyl group and 9-anthracenyl group.

Examples of the divalent hydrocarbon group include groups having a structure established by subtracting one hydrogen atom from the aforementioned groups.

### [Substituent Group]

Examples of the above-mentioned substituent group include, but are particularly limited to, a hydrocarbon group, an oxygen-containing group, a nitrogen-containing group, a sulfur-containing group, a phosphorus-containing group, and a halogen. The substituent group also includes a group to which these substituent groups are connected.

Examples of the above-mentioned hydrocarbon group include those listed in the section of [Substituent Group].

Examples of the oxygen-containing group include, but are not particularly limited to, a hydroxy group, an alkoxy group, an acetoxy group, an acetyl group, an aldehyde group, a carboxy group, a carboxylate group, a urea group, a urethane group, an amide group, an imide group, an ether group, a carbonyl group, an ester group, an oxazole group, a morpholin group, a carbamate group, a carbamic acid group, a carbamoyl group, a polyoxyethylene group, a tocopheryl group, a chroman group, a dihydropyran group, a glyceryl group, and a glyceryl ether group.

Examples of the nitrogen-containing group include, but are not particularly limited to, an amino group, a cyano group, a cyanato group, an isocyanate group, a nitro group, a nitroalkyl group, an amide group, a urea group, a urethane group, an imide group, a carbodiimide group, an azo group, a pyridine group, an imidazole group, a pyrrolidine group, a piperidine group, pyrroline group, a pyrazine group, a triazole group, an isoquinoline group, an oxazoline group, a thiazoline group, a morpholine group, a guanidine group, a pyrimidine group, a piperazine group, a triazine group, a quinoline group, an indole group, a quinoxaline group, an isooxazoline group, a primary amino group, a secondary amino group, a tertiary amino group, a quaternary ammonium group, and an aminoalkyl group.

Examples of the sulfur-containing group include, but are not particularly limited to, a sulfate group, a sulfonyl group, a sulfonate group, a mercapto group, a thioether group, a thiocarbonyl group, a thiourea group, a thiocarboxy group, a thiocarboxylate group, a dithiocarboxy group, a dithiocarboxylate group, a sulfuric ester, a thiophene group, a thiazole group, a thiol group, a sulfo group, a sulfide group, a disulfide group, a thioester group, a thioamide group, a thiocarbamate group, and a dithiocarbamate group, and esters thereof.

Examples of the phosphorus-containing group include, but are not particularly limited to, a phosphate group, a phosphorous acid group, a phosphonic acid group, a phosphinic acid group, a phosphonous acid group, a phosphinous acid group, a pyrophosphate group, a phosphate group, a phosphorous acid ester group, a phosphonic acid ester group, a pyrophosphate group and esters thereof.

Examples of the halogen include fluorine, chlorine, bromine, and iodine.

Examples of the organic group include a hydrocarbon group that may contain a substituent group, wherein the hydrocarbon moiety thereof may also contain an oxygen atom(s). Examples of the above-mentioned hydrocarbon group include those as referred to and set forth in the section of [Substituent Group]. The hydrocarbon group is preferably an aliphatic hydrocarbon group, among which a saturated aliphatic hydrocarbon group (such as an alkyl group) is more preferred. It is preferred in terms of activating interface that such an alkyl group be a group having 1 to 22 carbon atoms, more preferably 4 to 22 carbon atoms, even more preferably 8 to 22 carbon atoms, particularly preferably 12 to 22 carbon atoms, and the group may be branched or linear. Meanwhile, it is preferred in terms of readily allowing the additive of the present invention to be liquid at 25° C. that such an alkyl group be a group having 1 to 18 carbon atoms, more preferably 1 to 12 carbon atoms, and even more preferably 1 to 4 carbon atoms, and the group may be branched or linear. The aforementioned hydrocarbon groups may contain a substituent group(s), and examples of such substituent groups include, but are not particularly limited to, those listed in the [Substituent Group] as mentioned above. Among the substituent groups, preferred are those having an oxygen-containing group, among which a hydroxy group, a carboxy group, a carboxylate group, an ether group and an alkoxy group are more preferred. Among them, even more preferred are a hydroxy group, a carboxy group and a carboxylate group; and particularly preferred are a hydroxy group and a carboxy group.

The aforementioned hydrocarbon moiety may contain an oxygen atom(s), in which case such hydrocarbon moiety contains the aforementioned oxygen-containing group, and forms or contains, for example, an ether bond, a carbonyl group, a hydroxy group, a carboxylate group, an ester bond, an amide bond, a urea bond or a urethane bond. Therefore, the wording “hydrocarbon moiety contains an oxygen atom(s)” as used herein encompasses a case where the hydrocarbon moiety is interrupted by, or the hydrogen atom(s) thereof are substituted by a group serving as an oxygen atom-containing atom group that even may contain optionally a hetero atom(s) such as a nitrogen atom, or a case where the group is present at its base end.

It is preferred in terms of enhancing the efficacy of the present invention that at least one moiety in component (A) into which a functional group can be introduced (atoms contained in chemical structures as basic skeletons, such as a nitrogen moiety, and a carbon moiety composing a ring together with nitrogen) be substituted by a hydroxyaliphatic hydrocarbon group(s), and the rest of those moieties independently be hydrogen atoms, or substituted by an organic group(s) having a hydrogen-bonding functional group. Further, it is a preferable embodiment when the component (A) is an amine composed only of a hydroxyaliphatic hydrocarbon group(s) and hydrogen atom(s) directly bonded to nitrogen or an amine composed only of hydroxyaliphatic hydrocarbon groups.

As for the organic group having a hydrogen-bonding functional group(s) as mentioned above, examples of the hydrogen-bonding functional group include, but are not particularly limited to, an oxygen-containing group, a nitrogen-containing group, a sulfur-containing group, a phosphorus-containing group, and a hydrogen atom directly bonded to nitrogen.

It is preferred in terms of affinity to water and affinity to metallic coordination that a hydrogen-bonding functional group contained in component (A) be a hydroxy group, a carboxy group, a carboxylate group, an ester group, an ether group, an alkoxy group, or a hydrogen atom directly bonded to nitrogen. Among them, more preferred are a hydroxy group, a carboxy group, a carboxylate group, an ether group, an alkoxy group, and a hydrogen atom directly bonded to nitrogen; even more preferred are a hydroxy group, a carboxy group, a carboxylate group, and a hydrogen atom directly bonded to nitrogen; particularly preferred are a hydroxy group, a carboxy group, and a hydrogen atom directly bonded to nitrogen; and most preferred are a hydroxy group and a hydrogen atom directly bonded to nitrogen. A preferable example of the organic group having such hydrogen-bonding functional group includes a hydrocarbon group having a hydrogen-bonding functional group. Examples of the organic group having such hydrogen-bonding functional group include a hydrocarbon group containing a hydroxy group (hydroxy hydrocarbon group), a hydrocarbon group containing a carboxy group (carboxy hydrocarbon group), a hydrocarbon group containing a hydroxy group and a carboxy group (hydroxycarboxy hydrocarbon group), a hydrocarbon group including a carboxylate group, a hydrocarbon group including an ester group, a hydrocarbon group containing an ether group, and a hydrocarbon group containing an alkoxy group.

At least one moiety in the component (A) into which a functional group can be introduced (atoms contained in chemical structures as basic skeletons, such as a nitrogen moiety, and a carbon moiety composing a ring together with nitrogen) may be substituted by an organic group having no hydrogen-bonding functional group. Examples of such organic group include a hydrocarbon group. Examples of the hydrocarbon group include an alkyl group. It is preferred that such an alkyl group be a linear or branched group having 1 to 22 carbon atoms, more preferably a linear or branched group having 1 to 18 carbon atoms, even more preferably a linear or branched group having 1 to 12 carbon atoms, particularly preferably a linear or branched group having 1 to 8 carbon atoms, and most preferably a linear or branched group having 1 to 6 carbon atoms.

A preferable embodiment of the component (A) includes an amine compound wherein 1 to 3 linear monohydroxyaliphatic hydrocarbon groups having one hydroxy group are bonded to a nitrogen atom whose remaining bonding site(s) is/are bonded to a hydrogen atom(s). Another preferable embodiment thereof includes an amine compound wherein 1 to 3 linear monohydroxyaliphatic hydrocarbon groups having one hydroxy group and 1 to 4 carbon atoms are bonded to a nitrogen atom whose remaining bonding site(s) is/are bonded to a hydrogen atom(s). Another preferable embodiment thereof includes an amine compound wherein one branched aliphatic hydrocarbon groups having 1 to 3 hydroxy groups is bonded to a nitrogen atom whose remaining bonding sites are bonded to hydrogen atoms. Another preferable embodiment thereof includes an amine compound wherein one branched aliphatic hydrocarbon group having 1 to 3 hydroxy groups and 3 to 5 carbon atoms is bonded to a nitrogen atom whose remaining bonding sites are bonded to hydrogen atoms. Another preferable embodiment thereof includes an amine compound having three hydroxy groups in one molecule, wherein 1 to 3 hydroxyaliphatic hydrocarbon groups are bonded to a nitrogen atom whose remaining bonding sites is/are bonded to a hydrogen atom(s).

In terms of usage and emulsification capability, it is more preferred that the component (A) be a monoethanolamine, diethanolamine, triethanolamine, 2-amino-2-hydroxymethyl-1,3-propanediol, 2-amino-1,3-propanediol, 2-amino-2-ethyl-1,3-propanediol or 2-amino-2-methyl-1,3-propanediol, among which 2-amino-2-hydroxymethyl-1,3-propanediol is even more preferred. Further, in terms of safety, it is more preferred that the component (A) be a diethanolamine, triethanolamine, 2-amino-2-hydroxymethyl-1,3-propanediol, 2-amino-1,3-propanediol, 2-amino-2-ethyl-1,3-propanediol or 2-amino-2-methyl-1,3-propanediol.

When component (A) is an ammonium compound, examples of the anions include, but are not particularly limited to, a hydroxide anion, a halogen-based anion, a sulfur-based anion, a phosphorus-based anion, a cyano-based anion, boron-based anion, a fluorine-based anion, a nitrogen oxide-based anion and a carboxylic acid anion, among which preferred is a hydroxide anion.

When the ammonium compound or amine of the component (A) is an amine compound, the aforementioned component (A) is preferably an amine compound represented by the following formula (I).

[Chemical formula 2]

N[R¹]ₘ[R²]₃₋ₘ (I)

In the formula, each R¹ independently represents a hydroxyaliphatic hydrocarbon group in which there is at least one hydroxy group, in which a hydrocarbon moiety thereof has 1 to 22 carbon atoms and optionally contains an oxygen atom(s), and in which each R₂ independently represents a hydrogen atom or an organic group having 1 to 22 carbon atoms, and m represents an integer of 0 to 3.

It is preferred that m in the formula (I) represent an integer of 1 to 2.

It is preferred that all of the R²s in the formula (I) be hydrogen atoms.

It is preferred that the hydroxyaliphatic hydrocarbon group in the formula (I) be a saturated hydroxyaliphatic hydrocarbon group whose hydrocarbon moiety has 1 to 22 carbon atoms and optionally contains an oxygen atom.

It is preferred that at least one of the R¹s in the formula (I) be a saturated hydroxyaliphatic hydrocarbon group having 1 to 3 hydroxy groups in which a hydrocarbon moiety thereof has 1 to 22 carbon atoms, said hydrocarbon moiety optionally containing an oxygen atom.

It is preferred that at least one of the R¹s in the formula (I) be a saturated hydroxyaliphatic hydrocarbon group having one hydroxy group in which a hydrocarbon moiety thereof has 1 to 22 carbon atoms, said hydrocarbon moiety optionally containing an oxygen atom.

It is preferred that at least one of the R¹s in the formula (I) be a branched hydroxyaliphatic hydrocarbon group having one or more hydroxy groups in which a hydrocarbon moiety thereof has 3 to 12 carbon atoms.

As for the respective preferable examples as shown in the formula (I), at least one of these combinations may be a more preferred embodiment(s).

A preferable embodiment of the formula (I) includes the one in which R¹ has one hydroxy group and is a linear monohydroxyaliphatic hydrocarbon group whose hydrocarbon moiety is linear and has 1 to 4 carbon atoms and in which all of the R²s are hydrogen atoms and m is an integer of 1 to 3. Another preferable embodiment thereof includes the one in which R¹ has 1 to 3 hydroxy groups and is a saturated hydroxyaliphatic hydrocarbon group whose hydrocarbon moiety is branched and has 3 to 5 carbon atoms and in which all of the R²s are hydrogen atoms and m is an integer of 1.

When the ammonium compound or amine of the component (A) is an ammonium compound, the aforementioned component (A) is preferably an ammonium compound represented by the following formula (II).

[Chemical formula 3]

N⁺[R³]ₙ[R⁴]₄₋ₙX⁻ (II)

In the formula, each R³ independently represents a hydroxyaliphatic hydrocarbon group in which there is at least one hydroxy group in which a hydrocarbon moiety thereof has 1 to 22 carbon atoms and optionally contains an oxygen atom(s), and in which each R⁴ independently represents a hydrogen atom or an organic group having 1 to 22 carbon atoms, and n represents an integer of 0 to 3. X⁻ represents an anion.

It is preferred in the formula (II) that the anion represented by X⁻ be a hydroxide ion.

It is preferred in the formula (II) that n be an integer of 1 to 3.

It is preferred in the formula (II) that all of the R¹s be hydrogen atoms.

It is preferred in the formula (II) that the hydroxyaliphatic hydrocarbon group represents a saturated hydroxyaliphatic hydrocarbon group whose hydrocarbon moiety has 1 to 22 carbon atoms, said hydrocarbon moiety optionally containing an oxygen atom.

It is preferred in the formula (II) that at least one of the R³s be a saturated hydroxyaliphatic hydrocarbon group having 1 to 3 hydroxy groups in which a hydrocarbon moiety thereof has 1 to 22 carbon atoms, said hydrocarbon moiety optionally containing an oxygen atom.

It is preferred in the formula (II) that at least one of the R³s be a saturated hydroxyaliphatic hydrocarbon group having one hydroxy group in which a hydrocarbon moiety thereof has 1 to 22 carbon atoms, said hydrocarbon moiety optionally containing an oxygen atom.

It is preferred in the formula (II) that at least one of the R¹s is a branched hydroxyaliphatic hydrocarbon group having one hydroxy group in which a hydrocarbon moiety thereof has 3 to 12 carbon atoms.

As for the respective preferable examples as shown in the formula (II), at least one of these combinations may be a more preferred embodiment(s).

A preferable embodiment of the additive according to the present invention includes an organic ammonium salt formed by the components (A) and (B).

The additive according to the present invention preferably includes an organic salt formed by: a cation originated from the component (A); and an anion originated from an anionic residue of the component (B), wherein said cation optionally contains a cationic residue of the component (B).

The cation is preferably an ammonium cation.

The residues in component (B) as used herein refer to atoms or atomic groups without a charge, and those having a charge to be cations are referred to as cationic residues while those to be anions are referred to as anionic residues.

According to the present invention, an unsaturated or branched aliphatic acid having 8 to 22 carbon atoms or a salt thereof in the component (B) has cationic and anionic residues. The cationic residues are hydrogen atoms, or groups (atom groups) that are bonded to a nitrogen atom of the component (A) to be hydrogen-bonding functional groups or organic groups. It is preferred that the acid of the component (B) is a compound composed of a hydrogen atom(s), serving as a proton, and an anionic residue(s).

In the present invention, the organic ammonium salt contains an organic cation or NH₄⁺ with a nitrogen atom being an ion center, and an organic anion. The salt particularly contains an organic cation and an organic anion. The term “organic” as used herein refers to those having hydrogen(s) and carbon(s) as element(s).

Examples of the cation originated from the component (A) include an ammonium cation (NH₄⁺, a primary ammonium cation, a secondary ammonium cation, a tertiary ammonium cation) such as, although not limited to the following, amines originated from cyclic amines such as an imidazolium cation, pyridinium cation, pyrrolidinium cation, piperidinium cation, pyrrolinium cation, pyrazinium cation, triazolium cation, isoquinolinium cation, oxazolinium cation, thiazolinium cation, morpholinium cation, guanidium cation, pyrimidinium cation, piperazinium cation, triadinium cation, quinolinium cation, indolinium cation, quinoxalinium cation, isooxazolium cation, and cationic amino acids. Among them, preferred are an ammonium cation, an imidazolium cation, a pyridinium cation, a pyrrolidinium cation, a piperidinium cation and a morpholinium cation; more preferred is an ammonium cation.

A cation of the ammonium salt formed by the components (A) and (B) is preferably a cation expressed by the following formula (III). In a case where the component (A) is an ammonium compound, the cation thereof is also preferably a cation expressed by the following formula (III).

[Chemical formula 4]

N⁺[R⁵]ₒ[R⁶]₄₋ₒ (III)

In the formula, each R⁵ independently represents a hydroxyaliphatic hydrocarbon group having at least one hydroxy group in which a hydrocarbon moiety thereof has 1 to 22 carbon atoms, and the hydrocarbon moiety optionally contains an oxygen atom(s), and wherein each R⁶ independently represents a hydrogen atom or an organic group having 1 to 22 carbon atoms, and o represents an integer of 0 to 4.

It is preferred in the formula (III) that o be an integer of 1 to 3.

It is preferred in the formula (III) that all of the R¹s be hydrogen atoms.

It is preferred in the formula (III) that the hydroxyaliphatic hydrocarbon group represents a saturated hydroxyaliphatic hydrocarbon group whose hydrocarbon moiety has 1 to 22 carbon atoms, said hydrocarbon moiety optionally containing an oxygen atom.

It is preferred in the formula (II) that at least one of the R¹s is a saturated hydroxyaliphatic hydrocarbon group having 1 to 3 hydroxy groups in which a hydrocarbon moiety thereof has 1 to 22 carbon atoms, said hydrocarbon moiety optionally containing an oxygen atom.

It is preferred in the formula (III) that at least one of the R¹s be a saturated hydroxyaliphatic hydrocarbon group having one hydroxy group in which a hydrocarbon moiety thereof has 1 to 22 carbon atoms, said hydrocarbon moiety optionally containing an oxygen atom.

It is preferred in the formula (III) that at least one of the R¹s is a branched hydroxyaliphatic hydrocarbon group having one hydroxy group in which a hydrocarbon moiety thereof has 3 to 12 carbon atoms.

When at least one of the R⁶s in the cation expressed by the formula (III) is a hydrogen atom, a proton corresponding to said hydrogen atom is preferably originated from the component (B).

As for the respective preferable examples as shown in the formula (III), at least one of these combinations may be a more preferred embodiment(s).

The cation expressed by the formula (III) includes, but are not particularly limited to, a cation in which a hydrogen ion(s) is/are bonded to the amine as expressed by the formula (I), and examples of such include, a monoethanol ammonium cation, a diethanol ammonium cation, a triethanol ammonium cation, a 2-amino-2-hydroxymethyl-1,3-propanediol ammonium cation, a 2-amino-1,3-propanediol ammonium cation, a 2-amino-2-ethyl-1,3-propanediol ammonium cation, a 2-amino-2-methyl-1,3-propanediol ammonium cation and a 2-amino-2-methyl-1-propanol ammonium cation. It is preferred that the cation be a 2-amino-2-hydroxymethyl-1,3-propanediol ammonium cation, 2-amino-1,3-propanediol ammonium cation, 2-amino-2-ethyl-1,3-propanediol ammonium cation, 2-amino-2-methyl-1,3-propanediol ammonium cation or 2-amino-2-methyl-1-propanol ammonium cation in terms of the enhanced affinity to the water phase, enhanced emulsion stability, improved feelings of use when it is used in a creamy composition and achieving a lubricity when used in a lubricant composition where it has enhanced emulsion stability during operation and does not collapse its emulsion to form two layers. Among them, a 2-amino-2-hydroxymethyl-1,3-propanediol ammonium cation may more preferably be used.

In the additive of the present invention, the component (B) may be an unsaturated or branched aliphatic acid having 8 to 22 carbon atoms or a salt thereof. The number of carbon atoms in the aliphatic acid as used herein include the number of carbon atoms in carboxy group(s). The aliphatic acid may have one or more carboxy groups or have a hydroxy group(s).

The unsaturated aliphatic acid having 8 to 22 carbon atoms may be linear or branched, and has preferably 8 to 18, more preferably 12 to 18 carbon atoms. Specific examples thereof include, for example, an octenoic acid, nonenoic acid, decenoic acid, undecenoic acid, undecylenic acid, dodecenoic acid, tridecenoic acid, tetradecenoic acid, myristoleic acid, pentadecenoic acid, hexadecenoic acid, palmitoleic acid, sapienic acid, heptadecenoic acid, octadecenoic acid, oleic acid, elaidin acid, vaccenic acid, nonadecenic acid, icosenoic acid, gadoleic acid, henicosenoic acid, docosenoic acid, erucic acid, octadienoic acid, nonadienoic acid, decadienoic acid, undecadienoic acid, dodecadienoic acid, tridecadienoic acid, tetradecadienoic acid, pentadecadienoic acid, hexadecadienoic acid, heptadecadienoic acid, octadecadienoic acid, linoleic acid, nonadecadienoic acid, icosadienoic acid, henicosadienoic acid, docosadienoic acid, octatrienoic acid, nonatrienoic acid, decatrienoic acid, undecatrienoic acid, dodecatrienoic acid, trideca trienoic acid, tetradecatrienoic acid, pentadecatrienoic acid, hexadecatrienoic acid, undecatetraenoic acid, dodecatetraenoic acid, tridecatetraenoic acid, tetradecatetraenoic acid, pentadecatetraenoic acid, hexadecatetraenoic acid, hexadecatetraenoic acid, heptadecatetraenoic acid, octadecatetraenoic acid, stearidonic acid, nonadecatetraenoic acid, icosatetraenoic acid, arachidonic acid, henicosatetraenoic acid, docosatetraenoic acid, adrenic acid, undecapentaenoic acid, dodecapentaenoic acid, tridecapentaenoic acid, tetradecapentaenoic acid, pentadecapentaenoic acid, hexadecapentaenoic acid, heptadecapentaenoic acid, octadecapentaenoic acid, boseopentaenoic acid, nonadecapentaenoic acid, icosapentaenoic acid, henicosapentaenoic acid, docosapentaenoic acid, clupanodonic acid, tridecahexaenoic acid, tetradecahexaenoic acid, pentadecahexaenoic acid, hexadecahexaenoic acid, heptadecahexaenoic acid, octadeca hexaenoic acid, boseohexaenoic acid, nonadecahexaenoic acid, icosahexaenoic acid, henicosahexaenoic acid, docosahexaenoic acid and their salts (such as an alkali metal cation, alkaline earth metal cation or ammonium cation). Any one of them may be used alone, or two or more of them may be used in combination. Particularly, in terms of surfactant functionality, an unsaturated aliphatic acid having 8 to 18 carbon atoms is preferred, while in terms of safety and availability, more preferred are an oleic acid, linoleic acid, linolenic acid and docosahexaenoic acid, among which oleic acid and linoleic acid are even more preferred, and oleic acid is particularly preferred.

The unsaturated aliphatic acid having 8 to 18 carbon atoms may be either a saturated or unsaturated aliphatic acid having preferably 8 to 18, more preferably 12 to 18 carbon atoms. Specific examples thereof include, for example, an ethylhexanoic acid, propyl pentanoic acid, butylbutanoic acid, dimethylhexanoic acid, methylheptanoic acid, propylpentanoic acid, hexyldecanoic acid (isopalmitic acid) and heptylundecanoic acid (isostearic acid). Among them, preferred are saturated fatty acids, among which heptylundecanoic acid (isostearic acid) and hexyldecanoic acid (isopalmitic acid) are more preferred, and heptylundecanoic acid (isostearic acid) is even more preferred.

When the component (B) is an unsaturated or branched aliphatic acid, a surfactant layer whose membrane viscoelasticity is greater (deformable and uncollapsible) than that of a saturated linear aliphatic acid is formed. The layer has an enhanced membrane viscoelasticity, and therefore is excellent in the feeling of use such as stretchability, skin compatibility and moist feeling when it is applied to, for example, cosmetics. Further, when it is applied for the purpose of a lubricant, it has enhanced emulsion stability during operation and does not collapse its emulsion to form two layers, which therefore makes it favorably used for achieving lubricity.

The unsaturated aliphatic acid or the branched aliphatic may be in the form of a mixture with a linear saturated aliphatic acid. When a mixed aliphatic acid is used, it is preferred that the unsaturated and branched aliphatic acids constitute the main components of which the content percentage of the unsaturated and branched aliphatic acids may be 10 to 100 wt. %, preferably 20 to 100 wt. %, more preferably 30 to 100 wt. %.

Examples of the salts of the component (B) include a salt of an anion obtained by dissociating a proton from the above-listed aliphatic acids and a cation (such as an alkali metal cation, an alkali earth metal cation and an ammonium cation).

Preferable combinations of the components (A) and (B), in terms of the emulsification functionality, feeling of use of the emulsion composition and lubricity functionality of the lubricant composition, include a combination of a cation of a liner or branched saturated hydroxyaliphatic hydrocarbon ammonium cation and an anion of an unsaturated or branched aliphatic acid anion, among which a combination of a cation of a branched saturated hydroxyaliphatic hydrocarbon ammonium cation and an anion of an unsaturated or branched aliphatic acid is more preferred.

In terms of the emulsification functionality and feeling of use of the emulsion composition, preferable combinations of the components (A) and (B) that form an organic ammonium salt include, for example, a combination of a cation of an monoethanol ammonium cation, triethanol ammonium cation, 2-amino-2-hydroxymethyl-1,3-propanediol ammonium cation, 2-amino-1,3-propanediol ammonium cation, 2-amino-2-ethyl-1,3-propanediol ammonium cation, 2-amino-2-methyl-1,3-propanediol ammonium cation or 2-amino-2-methyl-1-propanol ammonium cation and an anion of olein acid anion or isostearic acid anion, among which a combination of a cation of a 2-amino-2-hydroxymethyl-1,3-propanediol ammonium cation, 2-amino-1,3-propanediol ammonium cation, 2-amino-2-ethyl-1,3-propanediol ammonium cation, 2-amino-2-methyl-1,3-propanediol ammonium cation or 2-amino-2-methyl-1-propanol ammonium cation and an anion of olein acid anion or isostearic acid anion of the components (A) and (B) that forms an organic ammonium salt is more preferred. In terms of the lubricating functionality of the lubricant composition, preferable combinations of the components (A) and (B) that form an organic ammonium salt include, for example, a combination of a cation of a triethanol ammonium cation, 2-amino-2-hydroxymethyl-1,3-propanediol ammonium cation, 2-amino-1,3-propanediol ammonium cation or 2-amino-2-ethyl-1,3-propanediol ammonium cation and an anion of olein acid or isostearic acid, among which a combination of a cation of an 2-amino-2-hydroxymethyl-1,3-propanediol ammonium cation, 2-amino-1,3-propanediol ammonium cation or 2-amino-2-ethyl-1,3-propanediol ammonium cation and an anion of olein acid or isostearic acid of the components (A) and (B) that forms an organic ammonium salt is more preferred.

As for the additive of the present invention, it may be provided that the mixture or salt of the components (A) and (B) is in an anhydrous state (anhydride) or a hydrate that has absorbed water in the air. A hydrate refers to a compound whose moisture rate has reached a saturated state after being left in the air at 25° C. and absorbing the water therein. A compound that does not absorb water when left in the air at 25° C. has no hydrate and is an anhydride.

As for the additive of the present invention, an anhydride and hydrate of the mixture or salt of the components (A) and (B) may be either a liquid or a solid at 25° C. However, it is preferred that the anhydride and/or hydrate thereof be liquid at 25° C. and that the coagulation point of the anhydride and/or hydrate be less than 25° C., more preferably be −5° C., even more preferably be −10° C. in terms of exhibiting their effects to the whole range of application when of the mixture or salt of the components (A) and (B) is liquid at near room temperature after a solvent such as water is evaporated.

As for the additive according to the present invention, the compounding molar ratio between the component (A) and the component (B) is not particularly limited but may be 1:99 to 99:1, preferably be 1:9 to 9:1, more preferably be 1:5 to 5:1, even more preferably be 1:2 to 2:1, and particularly preferably be 1:1.

The additive may take any form which is not particularly limited but examples of such include an additive composed only of the components (A) and (B), a solution in which the components (A) and (B) are dissolved into a solvent, a mixture with a further component(s), an emulsion composition and a dispersing liquid where the components (A) and (B) are used as dispersants.

Examples of the solvent for the above-mentioned solution include, but are not particularly limited to, water, methanol, ethanol, propanol, isopropanol, butanol, ethylene glycol, propylene glycol, 1,3-butylene glycol, diethylene glycol, dipropylene glycol, isoprene glycol, hexylene glycol, glycerin, benzyl alcohol, methyl acetate, ethyl acetate, isopropyl acetate, ethyl ether, acetone, toluene, hexane, heptane and acetonitrile of which any one of them may be used alone, or two or more of them may be used in combination.

Examples of the further component include not only a surfactant and an oil agent but also, although not limited to the following, for example, a polymer compound (such as a cationic polymer and a water-soluble polymer), a viscosity regulator, resin particles, a gloss imparting agent, a higher alcohol, a multivalent alcohol, a higher fatty acid, amidoamines, a hydrocarbon, a wax, esters, a silicone derivative, a physiologically active ingredient, extracts, an antioxidant, a sequestrant, a preservative, an ultraviolet absorber (organic and inorganic), a perfume, a moisturizer, carbons, metal oxides, minerals, salts, a neutralizer, a pH adjuster, a refrigerant, an insect repellent, an enzyme, a dye, an organic colorant, an inorganic colorant, a coloring agent, a pearling agent, a pearlizing agent, an anti-inflammatory agent, an antioxidant, a whitening agent, a wrinkle ameliorating agent, vitamins, amino acids, a hair growing agent, an antibacterial agent, a hormonal agent, a plant extract, a seaweed extract ingredient, a crude drug ingredient, an activator, a blood circulation promoter, organic modified clay minerals, an extreme pressure agent, an antiwear agent, a detergent dispersant, a viscosity index improver, a corrosion inhibitor, a rust inhibitor, a metal deactivator, an antifoaming agent, a fluid point depressant, a demulsifier, and an antifungal agent. Any one of them may be used alone, or two or more of them may be used in combination.

The content of the components (A) and (B) in the additive of the present invention may be, although not limited to the following, for example, 0.1 to 100 wt. %, 1 to 100 wt. %, 1 to 99 wt. % or 10 to 90 wt. % based on the total weight of the additive.

The additive of the present invention may be added thereto and suitably used as a component of the emulsion composition. The additive of the present invention may be added thereto for activating, for example, solid/liquid, gas/liquid, gas/solid, liquid/liquid and solid/solid interfaces.

Conventionally, surfactants have been often used for surface activation, which are used in combination by suitably selecting the HLB of the surfactant based on a ratio between water and oil agent of the subject emulsion composition. The additive of the present invention exhibits excellent emulsion stabilizing effects as the additive may be applied to a wide range of ratio between water and oil agent and contains the components (A) and (B) as ionizable compounds.

The additive of the present invention may be used for the proposes where a conventional surfactant is applied, and is excellent for obtaining a stable and uniform composition when the composition contains an oil agent. The additive is also excellent in emulsion stability in the case of an emulsion composition that contains water and an oil agent. The additive of the present invention may also be used for various proposes where an activation of an interface is required, and examples of such include, but are not particularly limited to, for example, a washing agent, a solubilizer, a softener for paper, cloth or hair, a hydrophilic agent, a penetrant, a bleach, an antiseptic/antibacterial agent, an insect repellent/antifungal agent, a surface treatment agent for organic/inorganic material, a dispersant, a suspension agent, a lubricant, an antifoaming agent, a foaming agent, a viscosity regulator, an antistatic agent, a dyeing auxiliary, a fluidity improving agent, a flow point depressant, a freezing-point depressant and a mold release agent.

Examples of the oil agents include, but are not particularly limited to, hydrocarbons, lipids, esters, fatty acids, higher alcohols, silicone oils, waxes, steroids, monomers, oligomers, fluid polymers (polymer compounds), silicone oils, alcohols, glycols, glycol ethers and cellosolves; these oil agents may be liquids, gels or solids at room temperature. Any one of them may be used alone, or two or more of them may be used in combination. Examples of the hydrocarbons include, but are not particularly limited to, mineral oils such as a liquid paraffin, paraffin, solid paraffin, light isoparaffin, light liquid isoparaffin, liquid isoparaffin, ceresin, microcrystalline wax, petroleum jelly, white petrolatum and mineral oil; and synthetic oils such as squalane, alkylbenzene, polyethylene wax, polypropylene wax, hydrogenated polyisobutene, ethylene-α-olefin-cooligomer and ethylene propylene polymer. Examples of the further synthetic oils include aromatic oils such as, although not limited to the following, for example, alkyl benzenes such as a monoalkyl benzene and a dialkyl benzene, and monoalkyl naphthalene, dialkyl naphthalene and polyalkyl naphthalene; and aliphatic oils such as, although not limited to the following, for example, a normal paraffin, isoparaffin, polybutene, polyisobutylene, polyalphaolefin (e.g. 1-octene oligomer, 1-decene oligomer, and ethylene-propylene oligomer) and its hydride, and a cooligomer of alphaolefin and ethylene. Examples of the ester-based oil include, but are not particularly limited to, diester oils such as dibutyl sebacate, di-2-ethylhexyl sebacate, dioctyl adipate, diisodecyl adipate, ditridecyl adipate, ditridecyl glutarate and methyl-acetylsinolate; or aromatic ester oils such as trioctyl trimellitate, tridecyl trimellitate and tetraoctyl pyromellitate; or even polyol ester oils such as trimethylolpropane caprylate, trimethylolpropane pelargonate, pentaerythritol-2-ethylhexanoate and pentaerythritol pelargonate; or yet a complex ester oil as an oligoester of a multivalent alcohol and a mixed fatty acid of dibasic acid-monobasic acid. Examples of the ether-based oil include, but are not particularly limited to, phenyl ether oils such as a monoalkyltriphenyl ether, alkyldiphenyl ether, dialkyldiphenyl ether, pentaphenyl ether, tetraphenyl ether, monoalkyl tetraphenyl ether and dialkyl tetraphenyl ether. Examples of the lipids include, but are not particularly limited to, an avocado oil, almond oil, flaxseed oil, olive oil, cacao oil, perilla oil, camellia oil, castor oil, sesame oil, wheat germ oil, rice germ oil, rice bran oil, sasanqua oil, safflower oil, soybean oil, evening primrose oil, camellia oil, corn oil, rapeseed oil, persic oil, palm kernel oil, coconut oil, palm oil, beef fat, pork fat, horse fat, sheep fat, shea fat, cacao fat, turtle oil, mink oil, egg yolk oil, purcellin oil, castor oil, jojoba oil, grape seed oil, macadamia nut oil, cotton seed oil, meadowfoam oil, coconut oil, peanut oil, cod liver oil, rose hip oil, hardened oil of beef fat, extremely hardened oil of beef fat, hardened castor oil and extremely hardened palm oil. Examples of the esters include, but are not particularly limited to, an alkyl ethylhexanoate esterstearic acid alkyl ester, palmitic acid alkyl ester, myristic acid alkyl ester, lauric acid alkyl ester, behenic acid alkyl ester, oleic acid alkyl ester, isostearic acid alkyl ester, 12-hydroxystearic acid alkyl ester, undecylenic acid alkyl ester, lanolin fatty acid alkyl ester, erucic acid alkyl ester, coconut oil fatty acid alkyl ester, stearoyloxystearic acid alkyl ester, isononanoic acid alkyl ester, dimethyloctanoic acid alkyl ester, octanoic acid alkyl ester, lactic acid alkyl ester, ethylhexanoic acid alkyl ester, neopentanoic acid alkyl ester, malic acid alkyl ester, phthalic acid alkyl ester, citric acid alkyl ester, malonic acid alkyl ester, adipic acid alkyl ester, ethylene glycol fatty acid ester, propanediol fatty acid ester, butanediol fatty acid ester, trimethylolpropane fatty acid ester, pentaerythritol fatty acid ester, polyglycerin fatty acid ester, trehalose fatty acid ester, pentylene glycol fatty acid ester and trimellitic acid tris(2-ethylhexyl). Examples of the fatty acids include, but are not particularly limited to, stearic acid, palmitic acid, myristic acid, lauric acid, behenic acid, oleic acid, isostearic acid, 12-hydroxystearic acid, undecylenic acid, lanolin fatty acid, erucic acid and stearoyloxystearic acid. Examples of the higher alcohols include, but are not particularly limited to, a lauryl alcohol, cetyl alcohol, cetostearyl alcohol, stearyl alcohol, oleyl alcohol, behenyl alcohol, lanolin alcohol, hexyldecanol, myristyl alcohol, aralkyl alcohol, phytosterol, isostearyl alcohol and octyldodecanol. Examples of the silicone oils include, but are not particularly limited to, an amino-modified silicone oil, epoxy-modified silicone oil, carboxyl-modified silicone oil, polyether-modified oil, polyglycerin-modified silicone oil, dimethylpolysiloxane, dimethylsilicone, polyether-modified silicone, methylphenylsilicone, alkyl-modified silicone, higher fatty acid-modified silicone, methylhydrogen silicone, fluorine-modified silicone, epoxy-modified silicone, carboxy-modified silicone, carbinol-modified silicone, amino-modified silicone, methylpolysiloxane, methylphenylpolysiloxane, silicone resin, dimethicone, methylhydrogenpolysiloxane, methylcyclopolysiloxane, octamethyltrisiloxane, tetramethylhexasiloxane and highly polymerized methylpolysiloxane. Examples of the waxes include, but are not particularly limited to, a Japan wax, beeswax, sumac wax, lacquer wax, sugarcane wax, palm wax, montan wax, carnauba wax, candelilla wax, rice bran wax, lanolin, spermaceti, reduced lanolin, liquid lanolin, hard lanolin, ceresin and ozokerite. Examples of the steroids include, but are not particularly limited to, a cholesterol, dihydrocholesterol and cholesterol fatty acid ester.

### (Emulsion Composition)

The emulsion composition according to the present invention contains the aforementioned additive of the present invention.

The emulsion composition according to the present invention encompasses not only a composition where water, an oil agent and the additive of the present invention in which the components (A) and (B) have been compounded beforehand are mixed during manufacturing, but also a composition in which the components (A) and (B) have been compounded before finally obtaining the emulsion composition.

The emulsion composition may be of any state which is not particularly limited but may be, for example, a water-in-oil (W/O type) emulsion composition, an oil-in-water (O/W type) emulsion composition, and a composite type emulsion (W/O/W type, O/W/O type). The emulsion composition may be of any exterior state which is not particularly limited but may be, for example, liquid or solid which may or may not have fluidity, and may be suitably selected according to an intended use or purpose.

The emulsion composition of the present invention includes the additive of the present invention, water and an oil agent as essential components to which a further component(s) may be added provided that the effects of the present invention will not be impaired by them. Examples of the oil agent include those as listed above.

Preferable examples of the emulsion composition include, for example, a cosmetic or a lubricant.

When the emulsion composition is used as a cosmetic, the additive according to the present invention contain not only the components (A) and (B) but also, although not limited to the following, for example, a solvent, a surfactant, an oil agent, a polymer (such as a cationic polymer and a water-soluble polymer), a water-soluble polymer, a viscosity regulator, resin particles, a gloss imparting agent, a higher alcohol, a multivalent alcohol, a higher fatty acid, amidoamines, a hydrocarbon, a wax, esters, a silicone derivative, a physiologically active ingredient, extracts, an antioxidant, a sequestrant, a preservative, an ultraviolet absorber (including those of organic and inorganic), a perfume, a moisturizer, carbons, metal oxides, minerals, salts, a neutralizer, a pH adjuster, a refrigerant, an insect repellent, an enzyme, a dye, an organic colorant, an inorganic colorant, a coloring agent, a pearling agent, a pearlizing agent, an anti-inflammatory agent, an antioxidant, a whitening agent, a wrinkle ameliorating agent, vitamins, amino acids, a hair growing agent, an antibacterial agent, a hormonal agent, a plant extract ingredient, a seaweed extract ingredient, a crude drug ingredient, an activator, a blood circulation promoter, and organic modified clay minerals. Any one of them may be used alone, or two or more of them may be used in combination.

When the emulsion composition is used as a lubricant, the additive according to the present invention contains not only the components (A) and (B) but also, although not limited to the following, for example, solvents such as water, alcohols, and organic solvents; further lubricants, and additives for improving or imparting functionalities, and specific examples of which include, but are not particularly limited to, a surfactant, an antioxidant, an oiliness agent, an extreme pressure agent, an antiwear agent, a detergent dispersant, a viscosity index improver, a corrosion inhibitor, a rust inhibitor, a metal deactivator, an antifoaming agent, a fluid point depressant, a demulsifier, and an antifungal agent. Any one of them may be used alone, or two or more of them may be used in combination.

Examples of the surfactant include, but are not particularly limited to, for example, an anionic surfactant, a non-ionic surfactant, a cationic surfactant and/or an ampholytic surfactant. Examples of the anionic surfactant include carboxylates such as a fatty acid soap, alkyl ether carboxylate, alkylene alkyl ether carboxylate, fatty acid amide ether carboxylate, acyl lactate, N-acylglutamate (e.g. triethanolamine cocoyl glutamate, sodium cocoyl glutamate), N-acylalaninate (e.g. sodium lauroylalaninate, sodium cocoylalaninate), N-acylmethyl-β-alaninate (e.g. sodium lauroylmethyl-β-alaninate), N-acylsarcosinate (e.g. sodium lauroylsarcosinate, triethanolamine lauroylsarcosinate), N-acylthreonine salt, N-acylglycine salt, N-acylaspartate, N-acylserine salt, N-acyl-@-amino acid salt, alkyl sulfoacetate and alkenyl sulfoacetate; sulfonates such as an alkane sulfonate, α-olefin sulfonate (e.g. sodium tetradecene sulfonate), α-sulfo fatty acid methyl ester salt, acyl isethionate, alkyl glycidyl ether sulfonate, alkyl sulfosuccinate, polyoxyalkylene alkyl sulfosuccinate (e.g. laureth sulfosuccinate 2Na, pareth sulfosuccinate 2Na), alkylbenzene sulfonate, alkylnaphthalene sulfonate, N-acyl taurate, N-acylmethyl taurate (e.g. sodium cocoyl methyl taurate), formalin condensation-based sulfonate, paraffin sulfonate, alkylamide sulfonate, alkenylamide sulfonate and alkylglyceryl ether sulfonate; sulfates such as an alkyl sulfate, alkenyl sulfate, alkyl ether sulfate, alkenyl ether sulfate, polyoxyalkylene alkyl ether sulfate (e.g. sodium polyoxyethylene lauryl ether sulfate), alkylaryl ether sulfate, fatty acid alkanolamide sulfate, fatty acid monoglyceride sulfate, polyoxyalkylene aliphatic amide ether sulfate and alkylglyceryl ether sulfate, and phosphates such as polyoxyalkylene alkyl ether phosphate (e.g. sodium polyoxyethylene lauryl ether phosphate, potassium polyoxyethylene lauryl ether phosphate), alkyl phosphate, alkylaryl ether phosphate and fatty acid amide ether phosphate. Although not particularly limited, examples of the nonionic surfactant include a glycerin fatty acid ester, polyoxyalkylene fatty acid ester, polyoxyalkylene alkyl ether, polyoxyalkylene alkyl phenyl ether, polyoxyalkylene (hardened) castor oil ester, sucrose fatty acid ester, polyglycerin alkyl ether, polyglycerin fatty acid ester, fatty acid alkanolamides such as alkyl alkanolamide, polyoxyethylene alkylalkanolamide and polyoxypropylene alkylalkanolamide, polyoxyalkylene glycerin fatty acid (mono/di/tri) ester, sorbitan fatty acid ester (such as sorbitan sesquiisostearate), polyoxyalkylene sorbitan fatty acid ester, and alkyl polyglycoside. Although not particularly limited, examples of the cationic surfactant include aliphatic amine salts and quaternary ammonium salts thereof, such as a primary amine salt, secondary amine salt, tertiary amine salt, aliphatic amide amine salt, aliphatic amide guanidium salt, quaternary ammonium salt (e.g. stearyltrimonium chloride, behentrimonium chloride), alkyl trialkylene glycol ammonium salt and alkyl ether ammonium salt; and cyclic quaternary ammonium salts such as benzalkonium salt, benzethonium salt, pyridinium salt and imidazolinium salt. Although not particularly limited, examples of the amphoteric surfactant include an alkylbetaine-type amphoteric surfactant, an amidebetaine-type amphoteric surfactant (e.g. coconut oil fatty acid amide propyl betaine, lauramidepropyl betaine, myristamidepropyl betaine, and palm kernel oil fatty acid amide propyl betaine), a sulfobetaine-type amphoteric surfactant (e.g. lauryl hydroxysulfobetaine), a phosphobetaine-type amphoteric surfactant, an imidazolinium betaine-type amphoteric surfactant (e.g. 2-alkyl-N-carboxymethyl-N-hydroxyethyl imidazolinium betaine), an alkylamine oxide-type amphoteric surfactant, an amino acid-type amphoteric surfactant and a phosphate ester-type amphoteric surfactant.

As for the emulsion composition of the present invention, a content of the additive of the present invention may be, although not limited to the following, 0.01 to 99.99 wt. %, preferably 0.1 to 99.9 wt. %, more preferably 0.1 to 99 wt. %, and even more preferably 0.01 to 20 wt. % based on the total weight of the emulsion composition. A surfactant may be parallelly used in addition to the additive of the present invention.

As for the emulsion composition of the present invention, a content of water may be, although not limited to the following, 0.1 to 99.9 wt. %, preferably 1 to 99 wt. %, more preferably 10 to 90 wt. %, and even more preferably 10 to 80 wt. % based on the total weight of the emulsion composition.

As for the emulsion composition of the present invention, a content of an oil agent may be, although not limited to the following, 0.1 to 99.9 wt. %, preferably 1 to 99 wt. %, more preferably 10 to 90 wt. %, and even more preferably 10 to 80 wt. % based on the total weight of the emulsion composition.

As for the emulsion composition of water-in-oil type (W/O type), a content of the additive of the present invention to water may be, although not limited to the following, 0.01 to 99.99 wt. %, preferably 0.1 to 99.9 wt. %, more preferably 0.1 to 99 wt. %, and even more preferably 1 to 20 wt. % based on the total weight of water.

As for the emulsion composition of oil-in-water type (O/W type), a content of the additive of the present invention to water may be, although not limited to the following, 0.01 to 99.99 wt. %, preferably 0.1 to 99.9 wt. %, more preferably 0.1 to 99 wt. %, and even more preferably 1 to 20 wt. % based on the total weight of water.

The emulsion composition containing the surfactant of the present invention may be used for, although not limited to the following, the application to skin such as cosmetics, a fragrance, a lubricant, a washing agent, a softener for paper, cloth or hair, a pharmaceutical, a pesticide, a paint, a food, a dye, a papermaking chemical, a swelling agent, an ink printing agent, a wetting agent, a surface treatment agent for organic/inorganic materials and a suspending agent.

When the composition is applied to the skin, it is excellent in stretchability, moist feeling and skin compatibility. When the composition is used as a lubricant, it is excellent in reducing the friction between metals.

### (Cosmetics)

The Cosmetic of the present invention is an emulsion composition to which the additive of the present invention is added. The form of the emulsion composition in the present invention is not particularly limited but may, for example, be a water-in-oil type (W/O type) emulsion composition, an oil-in-water (O/W type) emulsion composition, or a composite type emulsion (W/O/W type, O/W/O type), which is excellent in the stability of the emulsion composition, and useful since the composition is excellent in the stretchability on the skin or hair, moist feeling, and skin compatibility. These advantages are effectively taken when the composition is of W/O or O/W type, particularly of W/O type.

### (Lubricant)

The lubricant of the present invention is an emulsion composition to which the additive of the present invention is added. The form of the emulsion composition in the present invention is not particularly limited but may, for example, be a water-in-oil type (W/O type) emulsion composition, an oil-in-water (O/W type) emulsion composition, or a composite type emulsion (W/O/W type, O/W/O type), which is excellent in the stability of the emulsion composition, and has an advantage of reducing friction between metal materials when used as a lubricant. These advantages are effectively taken when the composition is of W/O or O/W type, particularly of W/O type.

Further, the emulsion composition may contain water to reduce the heat, flame and/or smoke which are/is generated during metallic processing, and also to reduce the viscosity to thereby improve the workability. Particularly, since the additive according to the present invention has a hydrogen-bonding group(s), it has a thermal conductivity that is superior to the water alone. Further, the additive may contain water to enhance the specific heat to thereby form an emulsion composition that is particularly excellent in cooling efficiency. Further, the hydrogen-bonding group has a good affinity (coordination) with, for example, metals, which contributes to the improvement in the lubricity.

Although the embodiments of the present invention have been described above, the present invention is not limited to these embodiments, and various modifications can be made without departing from the scope of the invention. Further, as for the respective preferable examples as shown in the formula (I), respective preferable examples as shown in the formula (II) and respective preferable examples as shown in the formula (III), at least one of these combinations may be a more preferred embodiment(s). As for the respective preferable examples as shown in the formulae (I) to (III), at least any of the combinations from all of them may be a more preferred embodiment(s) based on the results of the working examples. In addition, combinations thereof with the respective preferred examples shown in the anions, combinations thereof with the properties of mixtures or salts of components (A) and (B), and combinations thereof with the respective preferable examples of the polymer compound can be a more preferable embodiment based on the results of the working examples.

### Working Examples

The present invention will be described in more detail below with reference to working examples, but the present invention shall not be limited to these examples.

### (Additive)

Additives 1 to 17, 28, 29, 31 and 32 as shown in Tables 1 to 28 were prepared by mixing the components (A) and (B) as shown in the tables at the molar ratios as shown in the tables into water or alcohol solvent, and then removing the solvent, which were found to be liquid at 25° C.

As for the component (A), a reagent manufactured by KANTO CHEMICAL CO., INC (monoethanolamine, diethanolamine, triethanolamine, triethylamine, 2-amino-2-hydroxymethyl-1,3-propanediol, 2-amino-1,3-propanediol, 2-amino-2-ethyl-1,3-propanediol, 2-amino-2-methyl-1,3-propanediol, 2-amino-2-methyl-1-propanol) was used. As for the component (B), a reagent manufactured by Tokyo Chemical Industry Co., Ltd. (oleic acid, isostearic acid, lauric acid, stearic acid, behenic acid) was used.

As for the additive 18, a reagent manufactured by KANTO CHEMICAL CO., INC (sodium oleate) was used.

As for the additive 19, a reagent manufactured by KANTO CHEMICAL CO., INC (dodecyl sodium sulfate) was used.

As for the additive 20, a reagent manufactured by KANTO CHEMICAL CO., INC (Tween 20) was used.

As for the additive 21, a reagent manufactured by KANTO CHEMICAL CO., INC (Tween 40) was used.

As for the additive 22, a reagent manufactured by KANTO CHEMICAL CO., INC (Tween 60) was used.

As for the additive 23, a reagent manufactured by KANTO CHEMICAL CO., INC (Tween 85) was used.

As for the additive 24, a reagent manufactured by KANTO CHEMICAL CO., INC (Span 20) was used.

As for the additive 25, the one manufactured by Nikko Chemicals Co., Ltd. (Sorbitan Sesquiisostearate, NIKKOLSI-15RV) was used.

As for the additive 26, a reagent manufactured by KANTO CHEMICAL CO., INC (Span 80) was used.

As for the additive 27, the one manufactured by MIYOSHI OIL & FAT CO., LTD. (Non-ionic Surfactant based on polyoxyethylene alkylether, E-451D) was used.

As for the additive 30, the one manufactured by MIYOSHI OIL & FAT CO., LTD. (Glycerol Fatty Acid Ester) was used.

As for the oil agent, a reagent manufactured by FUJIFILM Wako Pure Chemical Corporation (Cetyl Ethylhexanoate) was used, a reagent manufactured by FUJIFILM Wako Pure Chemical Corporation (petrolatum) was used, and an ester oil manufactured by MIYOSHI OIL & FAT CO., LTD. (OTMP-300) was used.

The working examples and comparative examples using these additives were subjected to the following evaluations. The results are as shown in Tables 1 to 25.

### 1. Evaluation 1 of Composition Using Additive for Imparting Interfacial Activity.

The compositions of the working examples 1 to 16 and the comparative examples 1 to 11 were prepared. The components (A) and (B) of the present invention and a solvent were mixed with each other at the composition ratios as listed in Tables 1A, 1B, 2A, 2B, 3A and 3B, and the additives having the solvent distilled away and an oil agent were mixed at 80° C. and stirred, after which water was added thereinto and heated for 5 minutes while stirring the same to obtain an emulsion composition.

Moreover, a similar emulsion composition was obtained by adding the components (A) and (B) as the additives of the present invention in a separate manner into an oil agent such that the composition ratios thereof were as listed in Tables 1A and 1B, Tables 2A and 2B, and Tables 3A and 3B, mixing and stirring them at 80° C., after which water was added thereinto and heated for 5 minutes while stirring the same.

The emulsified states of the obtained emulsion compositions were identified by their appearance or the presence (absence) of fluidity. The presence (absence) of phase separation was identified by their appearance by which evaluation was performed by giving “O” to uniform compositions exhibiting no phase separation; and “X” to compositions exhibiting phase separations. It was determined in accordance with dye method that the emulsified states were of O/W type.

The obtained emulsion compositions were subjected to stability tests that were performed on the basis of the following evaluations 1 and 2. The evaluation 1 (centrifugalization) was performed with respect to the stabilities after performing centrifugations at (1) 16000 rpm/5 minutes, (2) 13500 rpm/5 minutes and (3) 13500 rpm/30 minutes on the bases of the following criteria.

### <Evaluation Criteria>

⊚: No separation was found at (1) to (3)
O: No separation was found at (1) and (2), and a separation was observed at (3)
Δ: No separation was found at (1), and separations were observed at (2) and (3)
X: Separations were observed at (1) to (3)

As for the evaluation 2. the resultant compositions were each left in a thermostatic bath controlled at 80° C. for 14 days to evaluate the changes in their emulsified states on the bases of the following criteria.

### <Criteria>

O: The emulsified state is maintained favourably.
X: The emulsified state is collapsed and the emulsion is in a separated state.

It was found from Tables 1A, 1B, 2A and 2B that the working examples 1 to 14 of the present invention resulted in emulsion compositions with reference to those of the comparative examples 1 to 4, 7, 8 and 10. Further, it was found that the compositions were excellent in emulsification stability compared to those of the comparative examples 5, 6, 9 and 11, which therefore indicates that the additive containing the components (A) and (B) exhibits an enhanced interfacial activity (emulsification) in the O/W type emulsified state.

When comparing the working examples 1 and 8 and the comparative examples 1 to 3 having a shared component (A) with a branched hydroxyaliphatic hydrocarbon group, the working examples 1 and 8 having the component (B) had an emulsion stability which is superior to the comparative examples 1 to 3 that do not have the component (B). This result suggests that the unsaturated or branched aliphatic acid having 8 to 22 carbon atoms in the component (B) contributed to the interfacial activity (emulsification) effect.

When comparing the working examples 1 and 6, the working examples 8 and 13 having the same number of (or three) hydroxy groups and a shared component (B), the working examples 1 and 8 having a branched hydroxyaliphatic hydrocarbon group in the component (A) had an emulsion stability which is superior to the working examples 6 and 13 that do not have a branched hydroxyaliphatic hydrocarbon group in the component (A). This result suggests that the branched hydroxyaliphatic hydrocarbon group in the component (A) contributed to the interfacial activity (emulsification) effect.

Among the working examples having a branched hydroxyaliphatic hydrocarbon group in the component (A), the examples 1 to 4 and 8 to 11 having two or more hydroxy groups in the branched hydroxyaliphatic hydrocarbon group had an emulsion stability which is superior to the working examples 5 and 12 having one hydroxy group. This result suggests that the number of hydroxy groups in the branched hydroxyaliphatic hydrocarbon group in the component (A) contributed to the interfacial activity (emulsification) effect, which suggests that those having two or more hydroxy groups are preferable.

Composition ratios of the components (A) and (B) were studied in Tables 3A and 3B. The results shows that the examples 15 and 16 having an additive composition ratio of 5 wt. % which is less than those of the working examples 1 and 8 resulted in emulsion compositions, which in turn suggests that the additives of the present invention exhibit an enhanced interfacial activity (emulsification) effect.

TABLE 1A

| | Additive | | | | | | | |
|---|---|---|---|---|---|---|---|---|
| | | 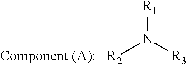 | | | | Com- pounding molar ratio | Moisture percent- age in hydrate | States of hydrates |
| | No. | R₁ | R₂ | R₃ | Component (B) | (A):(B) | (wt. %) | at 25° C. |
| Working example 1 | 1 | 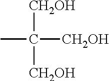 | H | H | CH₃(CH₂)₇ CH═CH(CH₂)₇COOH | 1:1 | 5.0 | Liquid |
| Working example 2 | 2 | 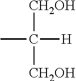 | H | H | CH₃(CH₂)₇ CH═CH(CH₂)₇COOH | 1:1 | 6.2 | Liquid |
| Working example 3 | 3 | 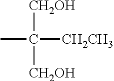 | H | H | CH₃(CH₂)₇ CH═CH(CH₂)₇COOH | 1:1 | 4.7 | Liquid |
| Working example 4 | 4 | 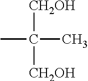 | H | H | CH₃(CH₂)₇ CH═CH(CH₂)₇COOH | 1:1 | 3.8 | Liquid |
| Working example 5 | 5 | 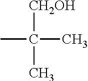 | H | H | CH₃(CH₂)₇ CH═CH(CH₂)₇COOH | 1:1 | 0.3 | Liquid |
| Working | 6 | (CH₂)₂OH | (CH₂)₂OH | (CH₂)₂OH | CH₃(CH₂)₇ CH═CH(CH₂)₇COOH | 1:1 | 5.6 | Liquid |
| example | | | | | | | | |
| 6 | | | | | | | | |
| Working | 7 | (CH₂)₂OH | H | H | CH₃(CH₂)₇ CH═CH(CH₂)₇COOH | 1:1 | 7.8 | Liquid |
| example | | | | | | | | |
| 7 | | | | | | | | |

TABLE 1B

| | Emulsion composition | | | | | | |
|---|---|---|---|---|---|---|---|
| | Composition ratio (wt. %) | | | | | Emulsion stability | |
| | | | | Surfactant | | | Evaluation 2 |
| | | | Oil agent | Sorbitan | Emulsified | | Left to stand |
| | | | Cetyl | sesqui- | state | Evaluation 1 | still at 50° C. |
| | Additive | Water | ethylhexanoate | isostearate | Appearance | Centrifugation | for 14 days |
| Working | 10 | 40 | 50 | 0 | ◯ | ⊚ | ◯ |
| example 1 | | | | | | | |
| Working | 10 | 40 | 50 | 0 | ◯ | ⊚ | ◯ |
| example 2 | | | | | | | |
| Working | 10 | 40 | 50 | 0 | ◯ | ⊚ | ◯ |
| example 3 | | | | | | | |
| Working | 10 | 40 | 50 | 0 | O | ⊚ | ◯ |
| example 4 | | | | | | | |
| Working | 10 | 40 | 50 | 0 | ◯ | ◯ | ◯ |
| example 5 | | | | | | | |
| Working | 10 | 40 | 50 | 0 | ◯ | ◯ | ◯ |
| example 6 | | | | | | | |
| Working | 10 | 40 | 50 | 0 | ◯ | ◯ | ◯ |
| example 7 | | | | | | | |

TABLE 2A

| | Additive | | | | | | | |
|---|---|---|---|---|---|---|---|---|
| | | 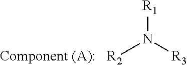 | | | | Com- pounding molar ratio | Moisture percent- age in hydrate | States of hy- drates |
| | No. | R₁ | R₂ | R₃ | Component (B) | (A):(B) | (wt. %) | at 25° C. |
| Working example 8 | 8 | 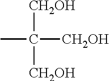 | H | H | CH₃(CH₂)₈CH((CH₂)₆CH₃)COOH | 1:1 | 15.6 | Liquid |
| Working example 9 | 9 | 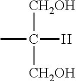 | H | H | CH₃(CH₂)₈CH((CH₂)₆CH₃)COOH | 1:1 | 6.7 | Liquid |
| Working example 10 | 10 | 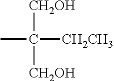 | H | H | CH₃(CH₂)₈CH((CH₂)₆CH₃)COOH | 1:1 | 4.9 | Liquid |
| Working example 11 | 11 | 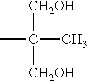 | H | H | CH₃(CH₂)₈CH((CH₂)₆CH₃)COOH | 1:1 | 4.8 | Liquid |
| Working example 12 | 12 | 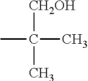 | H | H | CH₃(CH₂)₈CH((CH₂)₆CH₃)COOH | 1:1 | 0.3 | Liquid |
| Working | 13 | (CH₂)₂OH | (CH₂)₂OH | (CH₂)₂OH | CH₃(CH₂)₈CH((CH₂)₆CH₃)COOH | 1:1 | 7.0 | Liquid |
| example | | | | | | | | |
| 13 | | | | | | | | |
| Working | 14 | (CH₂)₂OH | H | H | CH₃(CH₂)₈CH((CH₂)₆CH₃)COOH | 1:1 | 10.3 | Liquid |
| example | | | | | | | | |
| 14 | | | | | | | | |

TABLE 2B

| | Emulsion composition | | | | | | |
|---|---|---|---|---|---|---|---|
| | Composition ratio (wt. %) | | | | | Emulsion stability | |
| | | | | Surfactant | | | Evaluation 2 |
| | | | Oil agent | Sorbitan | Emulsified | | Left to stand |
| | | | Cetyl | sesqui- | state | Evaluation 1 | still at 50° C. |
| | Additive | Water | ethylhexanoate | isostearate | Appearance | Centrifugation | for 14 days |
| Working | 10 | 40 | 50 | 0 | ◯ | ⊚ | ◯ |
| example 8 | | | | | | | |
| Working | 10 | 40 | 50 | 0 | ◯ | ⊚ | ◯ |
| example 9 | | | | | | | |
| Working | 10 | 40 | 50 | 0 | ◯ | ⊚ | ◯ |
| example 10 | | | | | | | |
| Working | 10 | 40 | 50 | 0 | ◯ | ⊚ | ◯ |
| example 11 | | | | | | | |
| Working | 10 | 40 | 50 | 0 | ◯ | ◯ | ◯ |
| example 12 | | | | | | | |
| Working | 10 | 40 | 50 | 0 | ◯ | ◯ | ◯ |
| example 13 | | | | | | | |
| Working | 10 | 40 | 50 | 0 | ◯ | ◯ | ◯ |
| example 14 | | | | | | | |

TABLE 3A

| | Additive | | | | | |
|---|---|---|---|---|---|---|
| | | 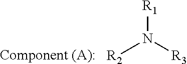 | | | | Com- pounding molar ratio |
| | No. | R₁ | R₂ | R₃ | Component (B) | (A):(B) |
| Working example 15 | 1 | 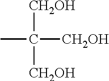 | H | H | CH₃(CH₂)₇ CH═CH(CH₂)₇COOH | 1:1 |
| Working example 16 | 8 | 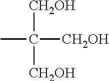 | H | H | CH₃(CH₂)₈CH((CH₂)₆CH₃)COOH | 1:1 |
| Com- parative example 1 | 15 | 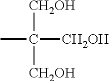 | H | H | CH₃(CH₂)₁₀COOH | 1:1 |
| Com- parative example 2 | 16 | 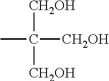 | H | H | CH₃(CH₂)₁₆COOH | 1:1 |
| Com- parative example 3 | 17 | 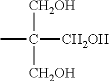 | H | H | CH₃(CH₂)₂₀COOH | 1:1 |

| | | |
|---|---|---|
| Comparative | 18 | Sodium oleate |
| example 4 | | |
| Comparative | 19 | Dodecyl sodium sulfate |
| example 5 | | |
| Comparative | 20 | Tween 20 |
| example 6 | | |
| Comparative | 21 | Tween 40 |
| example 7 | | |
| Comparative | 22 | Tween 60 |
| example 8 | | |
| Comparative | 23 | Tween 85 |
| example 9 | | |
| Comparative | 24 | Span 20 |
| example 10 | | |
| Comparative | 25 | Sorbitan sesquiisostearate |
| example 11 | | |

TABLE 3B

| | Emulsion composition | | | | | | |
|---|---|---|---|---|---|---|---|
| | Composition ratio (wt. %) | | | | | Emulsion stability | |
| | | | | Surfactant | | | Evaluation 2 |
| | | | Oil agent | Sorbitan | Emulsified | | Left to stand |
| | | | Cetyl | sesqui- | state | Evaluation 1 | still at 50° C. |
| | Additive | Water | ethylhexanoate | isostearate | Appearance | Centrifugation | for 14 days |
| Working | 5 | 40 | 55 | 0 | ◯ | ⊚ | ◯ |
| example 15 | | | | | | | |
| Working | 5 | 40 | 55 | 0 | ◯ | ⊚ | ◯ |
| example 16 | | | | | | | |
| Comparative | 10 | 40 | 50 | 0 | X | X | X |
| example 1 | | | | | | | |
| Comparative | 10 | 40 | 50 | 0 | X | X | X |
| example 2 | | | | | | | |
| Comparative | 10 | 40 | 50 | 0 | X | X | X |
| example 3 | | | | | | | |
| Comparative | 10 | 40 | 50 | 0 | X | X | X |
| example 4 | | | | | | | |
| Comparative | 10 | 40 | 50 | 0 | ◯ | X | X |
| example 5 | | | | | | | |
| Comparative | 10 | 40 | 50 | 0 | ◯ | X | X |
| example 6 | | | | | | | |
| Comparative | 10 | 40 | 50 | 0 | X | X | X |
| example 7 | | | | | | | |
| Comparative | 10 | 40 | 50 | 0 | X | X | X |
| example 8 | | | | | | | |
| Comparative | 10 | 40 | 50 | 0 | ◯ | Δ | X |
| example 9 | | | | | | | |
| Comparative | 10 | 40 | 50 | 0 | X | X | X |
| example 10 | | | | | | | |
| Comparative | 10 | 40 | 50 | 0 | ◯ | Δ | X |
| example 11 | | | | | | | |

### 2. Evaluation 1 of Composition in Parallel Use of Surfactant and Additive for Imparting Interfacial Activity

The compositions of the working examples 17 to 30 and the comparative examples 12 to 27 were prepared. The components (A) and (B) of the present invention and a solvent were mixed with each other at the composition ratios as listed in Tables 4A and 4B and Tables 5A and 5B, and the additive, having the solvent distilled away, an oil agent and a surfactant were mixed at 80° C. and stirred, after which water was added thereinto and heated for 5 minutes while stirring the same to obtain an emulsion composition.

A similar emulsion composition was obtained by adding the components (A) and (B) as the additives of the present invention in a separate manner into an oil agent and a surfactant such that the composition ratios thereof were as listed in Tables 4A and 4B and Tables 5A and 5B, mixing and stirring them at 80° C., after which water was added thereinto and heated for 5 minutes while stirring the same. The obtained emulsion compositions were observed with respect to their emulsified states and emulsion stabilities in a similar manner as in the section 1.

As shown from the results in Tables 4A, 4B, 5A and 5B, the working examples 17 to 30 of the present invention resulted in emulsion compositions with reference to the comparative examples 12 to 16, 18 and 20 to 24, and it was further found that these working examples were superior in terms of emulsion stability to the comparative examples 17, 19, 23 and 23 to 25. This result suggests that the additive of the present invention, containing the components (A) and (B), exhibits an enhanced interfacial activity (emulsification) effect.

Further, a tendency similar to the section 1 was observed in the correlation between the structure of the components (A) and (B) and the interfacial activity (emulsification) effects.

TABLE 4A

| | Additive | | | | | | Emulsion composition | | | | | | |
|---|---|---|---|---|---|---|---|---|---|---|---|---|---|
| | | | | | | Com- | Composition ratio (wt. %) | | | | | Emulsion stability | |
| | | 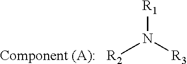 | | | | pound- ing molar ratio | | | Oil agent Petro- | Surfac- tant Sorbitan sesqui- | Emulsi- fied state Appear- | Evaluation 1 Cen- trifu- | Evaluation 2 Left to stand still at 50° C. for |
| | No. | R₁ | R₂ | R₃ | Component (B) | (A):(B) | Additive | Water | latum | isostearate | ance | gation | 14 days |
| Working example 17 | 1 | 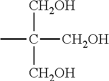 | H | H | CH₃(CH₂)₇ CH═CH(CH₂)₇COOH | 1:1 | 10 | 40 | 48 | 2 | ◯ | ⊚ | ◯ |
| Working example 18 | 2 | 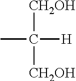 | H | H | CH₃(CH₂)₇ CH═CH(CH₂)₇COOH | 1:1 | 10 | 40 | 48 | 2 | ◯ | ⊚ | ◯ |
| Working example 19 | 3 | 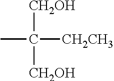 | H | H | CH₃(CH₂)₇ CH═CH(CH₂)₇COOH | 1:1 | 10 | 40 | 48 | 2 | ◯ | ⊚ | ◯ |
| Working example 20 | 4 | 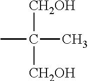 | H | H | CH₃(CH₂)₇ CH═CH(CH₂)₇COOH | 1:1 | 10 | 40 | 48 | 2 | ◯ | ⊚ | ◯ |
| Working example 21 | 5 | 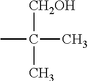 | H | H | CH₃(CH₂)₇ CH═CH(CH₂)₇COOH | 1:1 | 10 | 40 | 48 | 2 | ◯ | ◯ | ◯ |
| Working | 6 | (CH₂)₂OH | (CH₂)₂OH | (CH₂)₂OH | CH₃(CH₂)₇ CH═CH(CH₂)₇COOH | 1:1 | 10 | 40 | 48 | 2 | ◯ | ◯ | ◯ |
| example | | | | | | | | | | | | | |
| 22 | | | | | | | | | | | | | |
| Working | 7 | (CH₂)₂OH | H | H | CH₃(CH₂)₇ CH═CH(CH₂)₇COOH | 1:1 | 10 | 40 | 48 | 2 | ◯ | ◯ | ◯ |
| example | | | | | | | | | | | | | |
| 23 | | | | | | | | | | | | | |

TABLE 4B

| | | | | | | | | | | | Emulsion stability | |
|---|---|---|---|---|---|---|---|---|---|---|---|---|
| | | | | | | Emulsion composition | | | | | | Evalua- |
| Additive | | | | | | Composition ratio (wt. %) | | | | | | tion 2 |
| | 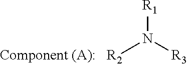 | | | Com- ponent | Com- pounding molar ratio | Addi- | | Oil agent Petro- | Surfac- tant Sorbitan sesqui- | Emulsi- fied state Appear- | Eval- uation 1 Centrifu- | Left to stand still at 50° C. for |
| No. | R₁ | R₂ | R₃ | (B) | (A):(B) | tive | Water | latum | isostearate | ance | gation | 14 days |

| | | | | | | | | | |
|---|---|---|---|---|---|---|---|---|---|
| Com- | 18 | Sodium oleate | 10 | 40 | 48 | 2 | X | X | X |
| parative | | | | | | | | | |
| example | | | | | | | | | |
| 12 | | | | | | | | | |
| Com- | 19 | Dodecyl sodium sulfate | 10 | 40 | 48 | 2 | X | X | X |
| parative | | | | | | | | | |
| example | | | | | | | | | |
| 13 | | | | | | | | | |
| Com- | 20 | Tween 20 | 10 | 40 | 48 | 2 | X | X | X |
| parative | | | | | | | | | |
| example | | | | | | | | | |
| 14 | | | | | | | | | |
| Com- | 21 | Tween 40 | 10 | 40 | 48 | 2 | X | X | X |
| parative | | | | | | | | | |
| example | | | | | | | | | |
| 15 | | | | | | | | | |
| Com- | 22 | Tween 60 | 10 | 40 | 48 | 2 | X | X | X |
| parative | | | | | | | | | |
| example | | | | | | | | | |
| 16 | | | | | | | | | |
| Com- | 23 | Tween 85 | 10 | 40 | 48 | 2 | ◯ | Δ | X |
| parative | | | | | | | | | |
| example | | | | | | | | | |
| 17 | | | | | | | | | |
| Com- | 24 | Span 20 | 10 | 40 | 48 | 2 | X | X | X |
| parative | | | | | | | | | |
| example | | | | | | | | | |
| 18 | | | | | | | | | |
| Com- | 25 | Sorbitan sesquiisostearate | 10 | 40 | 48 | 2 | ◯ | Δ | Δ |
| parative | | | | | | | | | |
| example | | | | | | | | | |
| 19 | | | | | | | | | |

TABLE 5A

| | | | | | | | Emulsion composition | | | | | | |
|---|---|---|---|---|---|---|---|---|---|---|---|---|---|
| | Additive | | | | | | Composition ratio (wt. %) | | | | | Emulsion stability | |
| | | 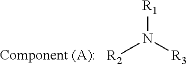 | | | | Com- pounding molar ratio | | | Oil agent Petro- | Surfac- tant Sorbitan sesqui- | Emulsi- fied state Appear- | Evalua- tion 1 Centrif- | Evaluation 2 Left to stand still at 50° C. for |
| | No. | R₁ | R₂ | R₃ | Component (B) | (A):(B) | Additive | Water | latum | isostearate | ance | ugation | 14 days |
| Working example 24 | 8 | 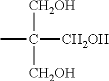 | H | H | CH₃(CH₂)₈CH((CH₂)₆CH₃)COOH | 1:1 | 10 | 60 | 28 | 2 | ◯ | ⊚ | ◯ |
| Working example 25 | 9 | 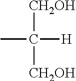 | H | H | CH₃(CH₂)₈CH((CH₂)₆CH₃)COOH | 1:1 | 10 | 60 | 28 | 2 | ◯ | ⊚ | ◯ |
| Working example 26 | 10 | 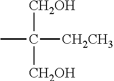 | H | H | CH₃(CH₂)₈CH((CH₂)₆CH₃)COOH | 1:1 | 10 | 60 | 28 | 2 | ◯ | ⊚ | ◯ |
| Working example 27 | 11 | 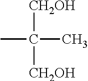 | H | H | CH₃(CH₂)₈CH((CH₂)₆CH₃)COOH | 1:1 | 10 | 60 | 28 | 2 | ◯ | ⊚ | ◯ |
| Working example 28 | 12 | 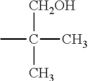 | H | H | CH₃(CH₂)₈CH((CH₂)₆CH₃)COOH | 1:1 | 10 | 60 | 28 | 2 | ◯ | ◯ | ◯ |
| Working | 13 | (CH₂)₂OH | (CH₂)₂OH | (CH₂)₂OH | CH₃(CH₂)₈CH((CH₂)₆CH₃)COOH | 1:1 | 10 | 60 | 28 | 2 | ◯ | ◯ | ◯ |
| example | | | | | | | | | | | | | |
| 29 | | | | | | | | | | | | | |
| Working | 14 | (CH₂)₂OH | H | H | CH₃(CH₂)₈CH((CH₂)₆CH₃)COOH | 1:1 | 10 | 60 | 28 | 2 | ◯ | ◯ | ◯ |
| example | | | | | | | | | | | | | |
| 30 | | | | | | | | | | | | | |

TABLE 5B

| | | | | Emulsion composition | | | | Emulsified | Emulsion | |
|---|---|---|---|---|---|---|---|---|---|---|
| Additive | | | | Composition ratio (wt. %) | | | | state | stability | |
| No. | 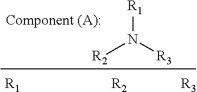 | Component (B) | Com- pounding molar ratio (A):(B) | Addi- tive | Water | Oil agent Pertro- latum | Surfactant Sorbitan sesqi- isostearate | Appear- ance | Evalua- tion 1 Centrif- ugation | Evalua- tion 2 Left to stand still at 50° C. for 14 days |

| | | | | | | | | | |
|---|---|---|---|---|---|---|---|---|---|
| Comparative | 18 | Sodium oleate | 10 | 60 | 28 | 2 | X | X | X |
| example | | | | | | | | | |
| 20 | | | | | | | | | |
| Comparative | 19 | Dodecyl sodium sulfate | 10 | 60 | 28 | 2 | X | X | X |
| example | | | | | | | | | |
| 21 | | | | | | | | | |
| Comparative | 20 | Tween 20 | 10 | 60 | 28 | 2 | X | X | X |
| example | | | | | | | | | |
| 22 | | | | | | | | | |
| Comparative | 21 | Tween 40 | 10 | 60 | 28 | 2 | X | X | X |
| example | | | | | | | | | |
| 23 | | | | | | | | | |
| Comparative | 22 | Tween 60 | 10 | 60 | 28 | 2 | X | X | X |
| example | | | | | | | | | |
| 24 | | | | | | | | | |
| Comparative | 23 | Tween 85 | 10 | 60 | 28 | 2 | ◯ | Δ | X |
| example | | | | | | | | | |
| 25 | | | | | | | | | |
| Comparative | 24 | Span 20 | 10 | 60 | 28 | 2 | ◯ | X | X |
| example | | | | | | | | | |
| 26 | | | | | | | | | |
| Comparative | 25 | Sorbitan sesquiisostearate | 10 | 60 | 28 | 2 | ◯ | Δ | Δ |
| example | | | | | | | | | |
| 27 | | | | | | | | | |

### 3. Feeling of Use 1 of Composition Utilizing Additive for Imparting Interfacial Activity

The emulsion composition of the working examples 31 to 44 and the comparative examples 28 to 31, having composition ratios as listed in Tables 6 and 7 and obtained by the prescription similar to the section 1, were each applied to the skin in a given amount to evaluate spreadability during the application, moist feeling and skin compatibility.

As for the spreadability during the application, evaluation was performed on a scale of 4 levels, in which ⊚ was given to examples where the emulsion composition had spread significantly, O was given to examples where the emulsion composition had spread, Δ was given to examples where the emulsion composition had spread less satisfactorily, X was given to examples where the emulsion composition had spread unsatisfactorily.

As for the moist feeling after the application, each composition was applied to the skin, and a feeling of the skin after spreading the composition was then evaluated on a scale of 4 levels, in which ⊚ was given to examples where a moist feeling was felt significantly, O was given to examples where a moist feeling was felt, Δ was given to examples where a moist feeling was less felt, and x was given to examples where no moist feeling was felt.

As for the skin compatibility after application, the evaluation was carried out based on a scale of 4 levels, in which ⊚ was given to examples where a favorable compatibility was observed, O was given to examples where a compatibility was observed, Δ was given to examples where a less favorable compatibility was observed, and X was given to examples where a poor compatibility was observed.

As can be seen from the results shown in Tables 6 and 7, it was observed that the working examples 31 to 44 of the present invention were superior in feeling of use compared to the comparative examples 28 to 31.

When comparing the working examples 31 to 35, 36 to 37, 38 to 42 and 43 to 44 having a shared component (B) of an unsaturated or branched aliphatic acid having 8 to 22 carbon atoms, the working examples 31 to 35 and 38 to 42 having a branched hydroxyaliphatic hydrocarbon group in the component (A) had an emulsion stability which is superior to the working examples 36, 37, 43 and 44 that do not have a branched hydroxyaliphatic hydrocarbon group in the component (A). This result suggests a contribution of the branched hydroxyaliphatic hydrocarbon group in the component (A).

Among the working examples having a branched hydroxyaliphatic hydrocarbon group in the component (A), the working examples 31 to 34 and 38 to 41 having two or more hydroxy groups in the branched hydroxyaliphatic hydrocarbon group in the component (A) had a feeling of use which is superior to the working examples 35 and 42 having one hydroxy group. This result suggests a contribution of the hydroxy group in the branched hydroxyaliphatic hydrocarbon group in the component (A), and it is also suggested that those having two or more hydroxy groups are excellent in feeling of use.

Composition ratios of the components (A) and (B) were studied in Table 8. The results shows that the examples 45 and 46 having an additive composition ratio of 5 wt. % which is less than the composition ratios of the working examples 31 and 38 resulted in feelings of use that were comparable to those having the ratio of 10 wt. %.

TABLE 6

| | | | | | | | Emulsion composition | | | | | | |
|---|---|---|---|---|---|---|---|---|---|---|---|---|---|
| | Additive | | | | | | Composition ratio (wt. %) | | | | Feeling of use | | |
| | | 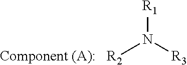 | | | | Com- pounding molar ratio | | | Oil agent Cetyl ethylhex- | Surfac- tant Sorbitan sesqui- | Stretch | Moist | Skin com- pat- ibil- |
| | No. | R₁ | R₂ | R₃ | Component (B) | (A):(B) | Additive | Water | anoate | isostearate | ability | feeling | ity |
| Working example 31 | 1 | 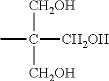 | H | H | CH₃(CH₂)₇ CH═CH(CH₂)₇COOH | 1:1 | 10 | 40 | 50 | 0 | ⊚ | ⊚ | ⊚ |
| Working example 32 | 2 | 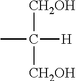 | H | H | CH₃(CH₂)₇ CH═CH(CH₂)₇COOH | 1:1 | 10 | 40 | 50 | 0 | ⊚ | ⊚ | ⊚ |
| Working example 33 | 3 | 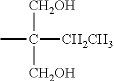 | H | H | CH₃(CH₂)₇ CH═CH(CH₂)₇COOH | 1:1 | 10 | 40 | 50 | 0 | ⊚ | ⊚ | ⊚ |
| Working example 34 | 4 | 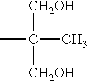 | H | H | CH₃(CH₂)₇ CH═CH(CH₂)₇COOH | 1:1 | 10 | 40 | 50 | 0 | ⊚ | ⊚ | ⊚ |
| Working example 35 | 5 | 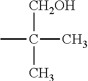 | H | H | CH₃(CH₂)₇ CH═CH(CH₂)₇COOH | 1:1 | 10 | 40 | 50 | 0 | ◯ | ◯ | ◯ |
| Working | 6 | (CH₂)₂OH | (CH₂)₂OH | (CH₂)₂OH | CH₃(CH₂)₇ CH═CH(CH₂)₇COOH | 1:1 | 10 | 40 | 50 | 0 | Δ | ◯ | ◯ |
| example | | | | | | | | | | | | | |
| 36 | | | | | | | | | | | | | |
| Working | 7 | (CH₂)₂OH | H | H | CH₃(CH₂)₇ CH═CH(CH₂)₇COOH | 1:1 | 10 | 40 | 50 | 0 | Δ | ◯ | ◯ |
| example | | | | | | | | | | | | | |
| 37 | | | | | | | | | | | | | |

| | | | | | | | | | |
|---|---|---|---|---|---|---|---|---|---|
| Comparative | 19 | Dodecyl sodium sulfate | 10 | 40 | 50 | 0 | Δ | X | X |
| example 28 | | | | | | | | | |
| Comparative | 20 | Tween 20 | 10 | 40 | 50 | 0 | Δ | Δ | Δ |
| example 29 | | | | | | | | | |
| Comparative | 23 | Tween 85 | 10 | 40 | 50 | 0 | Δ | Δ | Δ |
| example 30 | | | | | | | | | |
| Comparative | 25 | Sorbitan sesquiisostearate | 10 | 40 | 50 | 0 | Δ | Δ | Δ |
| example 31 | | | | | | | | | |

TABLE 7

| | | | | | | | Emulsion composition | | | | | | |
|---|---|---|---|---|---|---|---|---|---|---|---|---|---|
| | Additive | | | | | | Composition ratio (wt. %) | | | | Feeling of use | | |
| | | 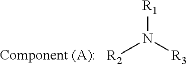 | | | | Com- pounding molar ratio | | | Oil agent Cetyl ethylhex- | Surfac- tant Sorbitan sesqui- | Stretch | Moist | Skin com- pat- ibil- |
| | No. | R₁ | R₂ | R₃ | Component (B) | (A):(B) | Additive | Water | anoate | isostearate | ability | feeling | ity |
| Working example 38 | 8 | 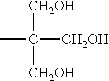 | H | H | CH₃(CH₂)₈CH((CH₂)₆CH₃)COOH | 1:1 | 10 | 40 | 50 | 0 | ⊚ | ⊚ | ⊚ |
| Working example 39 | 9 | 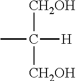 | H | H | CH₃(CH₂)₈CH((CH₂)₆CH₃)COOH | 1:1 | 10 | 40 | 50 | 0 | ⊚ | ⊚ | ⊚ |
| Working example 40 | 10 | 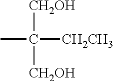 | H | H | CH₃(CH₂)₈CH((CH₂)₆CH₃)COOH | 1:1 | 10 | 40 | 50 | 0 | ⊚ | ⊚ | ⊚ |
| Working example 41 | 11 | 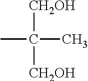 | H | H | CH₃(CH₂)₈CH((CH₂)₆CH₃)COOH | 1:1 | 10 | 40 | 50 | 0 | ⊚ | ⊚ | ⊚ |
| Working example 42 | 12 | 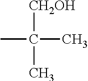 | H | H | CH₃(CH₂)₈CH((CH₂)₆CH₃)COOH | 1:1 | 10 | 40 | 50 | 0 | ◯ | ◯ | ◯ |
| Working | 13 | (CH₂)₂OH | (CH₂)₂OH | (CH₂)₂OH | CH₃(CH₂)₈CH((CH₂)₆CH₃)COOH | 1:1 | 10 | 40 | 50 | 0 | Δ | ◯ | ◯ |
| example | | | | | | | | | | | | | |
| 43 | | | | | | | | | | | | | |
| Working | 14 | (CH₂)₂OH | H | H | CH₃(CH₂)₈CH((CH₂)₆CH₃)COOH | 1:1 | 10 | 40 | 50 | 0 | Δ | ◯ | ◯ |
| example | | | | | | | | | | | | | |
| 44 | | | | | | | | | | | | | |

| | | | | | | | | | |
|---|---|---|---|---|---|---|---|---|---|
| Comparative | 19 | Dodecyl sodium sulfate | 10 | 40 | 50 | 0 | Δ | X | X |
| example 28 | | | | | | | | | |
| Comparative | 20 | Tween 20 | 10 | 40 | 50 | 0 | Δ | Δ | Δ |
| example 29 | | | | | | | | | |
| Comparative | 23 | Tween 85 | 10 | 40 | 50 | 0 | Δ | Δ | Δ |
| example 30 | | | | | | | | | |
| Comparative | 25 | Sorbitan sesquiisostearate | 10 | 40 | 50 | 0 | Δ | Δ | Δ |
| example 31 | | | | | | | | | |

TABLE 8

| | Additive | | | | | | | | | | | | |
|---|---|---|---|---|---|---|---|---|---|---|---|---|---|
| | | | | | | Com- | Emulsion composition | | | | | | |
| | | | | | | pound- | Composition ratio (wt. %) | | | | | | |
| | | | | | | ing | | | Oil agent | Surfactant | Feeling of use | | |
| | | Component (A): | | | | molar | | | Cetyl | Sorbitan | | | Skin |
| | | 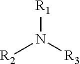 | | | | ratio | Addi- | Wa- | ethylhexa- | sesqui- | Stretch | Moist | compat- |
| | No. | R₁ | R₂ | R₃ | Component (B) | (A):(B) | tive | ter | noate | isostearate | ability | feeling | ibility |
| Working example 45 | 1 | 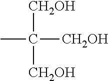 | H | H | CH₃(CH₂)₇ CH═CH(CH₂)₇COOH | 1:1 | 5 | 40 | 55 | 0 | ⊚ | ⊚ | ⊚ |
| Working example 46 | 8 | 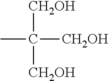 | H | H | CH₃(CH₂)₈CH ((CH₂)₆CH₃₎COOH | 1:1 | 5 | 40 | 55 | 0 | ⊚ | ⊚ | ⊚ |

### 4. Feeling of Use 1 of Composition in Parallel Use of Surfactant and Additive for Interfacial Surface Activation

Feelings of use for the emulsion compositions of the working examples 47 to 60 and the comparative examples 32 to 36 which were prepared in the same manner as those of the section 1 were evaluated on the basis of the criteria which were similar to those of the section 3.

As shown from the results in Tables 9 and 10, it was found that the working examples 47 to 60 were superior in terms of feeling of use to the comparative examples 32 to 36.

Further, a tendency similar to the section 3 was observed in the correlation between the structure of the components (A) and (B) and the feeling of use.

TABLE 9

| | | | | | | | Emulsion composition | | | | | | |
|---|---|---|---|---|---|---|---|---|---|---|---|---|---|
| | Additive | | | | | | Composition ratio (wt. %) | | | | Feeling of use | | |
| | | 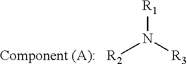 | | | | Compounding molar ratio | | | Oil agent | Surfac- tant Sorbitan sesqui- | Stretch | Moist | Skin com- pat- ibil- |
| | No. | R₁ | R₂ | R₃ | Component (B) | (A):(B) | Additive | Water | Petrolatum | isostearate | ability | feeling | ity |
| Working example 47 | 1 | 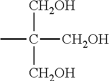 | H | H | CH₃(CH₂)₇ CH═CH(CH₂)₇COOH | 1:1 | 10 | 40 | 48 | 2 | ⊚ | ⊚ | ⊚ |
| Working example 48 | 2 | 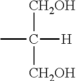 | H | H | CH₃(CH₂)₇ CH═CH(CH₂)₇COOH | 1:1 | 10 | 40 | 48 | 2 | ⊚ | ⊚ | ⊚ |
| Working example 49 | 3 | 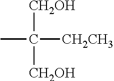 | H | H | CH₃(CH₂)₇ CH═CH(CH₂)₇COOH | 1:1 | 10 | 40 | 48 | 2 | ⊚ | ⊚ | ⊚ |
| Working example 50 | 4 | 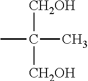 | H | H | CH₃(CH₂)₇ CH═CH(CH₂)₇COOH | 1:1 | 10 | 40 | 48 | 2 | ⊚ | ⊚ | ⊚ |
| Working example 51 | 5 | 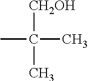 | H | H | CH₃(CH₂)₇ CH═CH(CH₂)₇COOH | 1:1 | 10 | 40 | 48 | 2 | ◯ | ◯ | ◯ |
| Working | 6 | (CH₂)₂OH | (CH₂)₂OH | (CH₂)₂OH | CH₃(CH₂)₇ CH═CH(CH₂)₇COOH | 1:1 | 10 | 40 | 48 | 2 | Δ | ◯ | ◯ |
| example | | | | | | | | | | | | | |
| 52 | | | | | | | | | | | | | |
| Working | 7 | (CH₂)₂OH | H | H | CH₃(CH₂)₇ CH═CH(CH₂)₇COOH | 1:1 | 10 | 40 | 48 | 2 | Δ | ◯ | ◯ |
| example | | | | | | | | | | | | | |
| 53 | | | | | | | | | | | | | |

| | | | | | | | | | |
|---|---|---|---|---|---|---|---|---|---|
| Compar- | 23 | Tween 85 | 10 | 40 | 48 | 2 | Δ | Δ | Δ |
| ative | | | | | | | | | |
| example | | | | | | | | | |
| 32 | | | | | | | | | |
| Compar- | 25 | Sorbitan sesquiisostearate | 10 | 40 | 48 | 2 | Δ | Δ | Δ |
| ative | | | | | | | | | |
| example | | | | | | | | | |
| 33 | | | | | | | | | |

TABLE 10

| | | | | | | | Emulsion composition | | | | | | |
|---|---|---|---|---|---|---|---|---|---|---|---|---|---|
| | Additive | | | | | | Composition ratio (wt. %) | | | | Feeling of use | | |
| | | 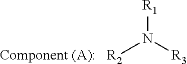 | | | | Compounding molar ratio | | | Oil agent Petro- | Surfac- tant Sorbitan sesqui- | Stretch | Moist | Skin com- pat- ibil- |
| | No. | R₁ | R₂ | R₃ | Component (B) | (A):(B) | Additive | Water | latum | isostearate | ability | feeling | ity |
| Working example 54 | 8 | 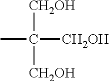 | H | H | CH₃(CH₂)₈CH((CH₂)₆CH₃)COOH | 1:1 | 10 | 60 | 28 | 2 | ⊚ | ⊚ | ⊚ |
| Working example 55 | 9 | 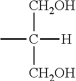 | H | H | CH₃(CH₂)₈CH((CH₂)₆CH₃)COOH | 1:1 | 10 | 60 | 28 | 2 | ⊚ | ⊚ | ⊚ |
| Working example 56 | 10 | 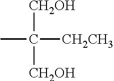 | H | H | CH₃(CH₂)₈CH((CH₂)₆CH₃)COOH | 1:1 | 10 | 60 | 28 | 2 | ⊚ | ⊚ | ⊚ |
| Working example 57 | 11 | 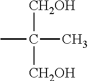 | H | H | CH₃(CH₂)₈CH((CH₂)₆CH₃)COOH | 1:1 | 10 | 60 | 28 | 2 | ⊚ | ⊚ | ⊚ |
| Working example 58 | 12 | 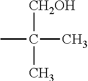 | H | H | CH₃(CH₂)₈CH((CH₂)₆CH₃)COOH | 1:1 | 10 | 60 | 28 | 2 | ◯ | ◯ | ◯ |
| Working | 13 | (CH₂)₂OH | (CH₂)₂OH | (CH₂)₂OH | CH₃(CH₂)₈CH((CH₂)₆CH₃)COOH | 1:1 | 10 | 60 | 28 | 2 | Δ | ◯ | ◯ |
| example | | | | | | | | | | | | | |
| 59 | | | | | | | | | | | | | |
| Working | 14 | (CH₂)₂OH | H | H | CH₃(CH₂)₈CH((CH₂)₆CH₃)COOH | 1:1 | 10 | 60 | 28 | 2 | Δ | ◯ | ◯ |
| example | | | | | | | | | | | | | |
| 60 | | | | | | | | | | | | | |

| | | | | | | | | | |
|---|---|---|---|---|---|---|---|---|---|
| Comparative | 23 | Tween 85 | 10 | 60 | 28 | 2 | Δ | Δ | Δ |
| example | | | | | | | | | |
| 34 | | | | | | | | | |
| Comparative | 24 | Span 20 | 10 | 60 | 28 | 2 | Δ | Δ | Δ |
| example | | | | | | | | | |
| 35 | | | | | | | | | |
| Comparative | 25 | Sorbitan sesquiisostearate | 10 | 60 | 28 | 2 | Δ | Δ | Δ |
| example | | | | | | | | | |
| 36 | | | | | | | | | |

### 5. Evaluation 2 of Composition Using Additive for Imparting Interfacial Activity.

The compositions of the working examples 61 to 74 and the comparative examples 37 to 40 were prepared. The components (A) and (B) of the present invention and a solvent were mixed with each other at the composition ratios as listed in Tables 11 and 12, and the additives, having the solvent distilled away, and an oil agent were mixed at 80° C. and stirred, after which water was added thereinto and heated for 5 minutes while stirring the same to obtain an emulsion composition. It was determined in accordance with the dye method that the emulsified states of the resultant compositions were of W/O type.

A similar emulsion composition was also obtained by adding the components (A) and (B) as the additives of the present invention in a separate manner into an oil agent such that the composition ratios thereof were as listed in Tables 11 and 12, mixing and stirring them at 80° C., after which water was added thereinto and heated for 5 minutes while stirring the same.

The emulsified state and the stability of the emulsion compositions were evaluated on the basis of the appearance of the resultant emulsion compositions, and the presence or absence of fluidity using the same criteria of the section 1.

As shown from the results in Tables 11 and 12, the working examples 61 to 74 resulted in emulsion compositions which were found to have emulsion stabilities that were superior to those of the comparative examples 37 to 40. This result suggests that the additive containing the components (A) and (B) of the present invention exhibits an enhanced interfacial activity (emulsification) effect even in a W/O type emulsified state.

TABLE 11

| | | | | | | | Emulsion composition | | | | | |
|---|---|---|---|---|---|---|---|---|---|---|---|---|
| | Additive | | | | | | Composition ratio (wt. %) | | | | | Emulsion |
| | | 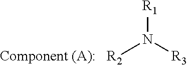 | | | | Compounding molar ratio | | | Oil agent Petro- | Surfac- tant Sorbitan sesqui- | Emulsified state | stability Evaluation 1 Centrifu- |
| | No. | R₁ | R₂ | R₃ | Component (B) | (A):(B) | Additive | Water | latum | isostearate | Appearance | gation |
| Working example 61 | 1 | 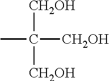 | H | H | CH₃(CH₂)₇ CH═CH(CH₂)₇COOH | 1:1 | 10 | 10 | 80 | 0 | ◯ | ◯ |
| Working example 62 | 2 | 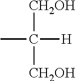 | H | H | CH₃(CH₂)₇ CH═CH(CH₂)₇COOH | 1:1 | 10 | 10 | 80 | 0 | ◯ | ◯ |
| Working example 63 | 3 | 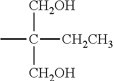 | H | H | CH₃(CH₂)₇ CH═CH(CH₂)₇COOH | 1:1 | 10 | 10 | 80 | 0 | ◯ | ◯ |
| Working example 64 | 4 | 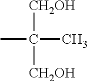 | H | H | CH₃(CH₂)₇ CH═CH(CH₂)₇COOH | 1:1 | 10 | 10 | 80 | 0 | ◯ | ◯ |
| Working example 65 | 5 | 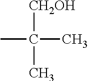 | H | H | CH₃(CH₂)₇ CH═CH(CH₂)₇COOH | 1:1 | 10 | 10 | 80 | 0 | ◯ | ◯ |
| Working | 6 | (CH₂)₂OH | (CH₂)₂OH | (CH₂)₂OH | CH₃(CH₂)₇ CH═CH(CH₂)₇COOH | 1:1 | 10 | 10 | 80 | 0 | ◯ | ◯ |
| example | | | | | | | | | | | | |
| 66 | | | | | | | | | | | | |
| Working | 7 | (CH₂)₂OH | H | H | CH₃(CH₂)₇ CH═CH(CH₂)₇COOH | 1:1 | 10 | 10 | 80 | 0 | ◯ | ◯ |
| example | | | | | | | | | | | | |
| 67 | | | | | | | | | | | | |

| | | | | | | | | |
|---|---|---|---|---|---|---|---|---|
| Comparative | 25 | Sorbitan sesquiisostearate | 10 | 10 | 80 | 0 | ◯ | X |
| example | | | | | | | | |
| 37 | | | | | | | | |
| Comparative | 26 | Span 80 | 10 | 10 | 80 | 0 | ◯ | X |
| example | | | | | | | | |
| 38 | | | | | | | | |

TABLE 12

| | | | | | | | Emulsion composition | | | | Emulsified | Emulsion |
|---|---|---|---|---|---|---|---|---|---|---|---|---|
| | Additive | | | | | | Composition ratio (wt. %) | | | | state | stability |
| | No. | 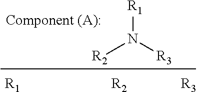 | | | Component (B) | Compounding molar ratio (A):(B) | Additive | Water | Oil agent Cetyl ethylhexanoate | Surfactant Sorbitan sesqiisostearate | Appearance | Evaluation 1 Centrifugation |
| Working Example 68 | 8 | 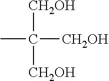 | H | H | CH₃(CH₂)₈CH((CH₂)₆CH₃)COOH | 1:1 | 10 | 10 | 80 | 0 | ◯ | ◯ |
| Working Example 69 | 9 | 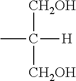 | H | H | CH₃(CH₂)₈CH((CH₂)₆CH₃)COOH | 1:1 | 10 | 10 | 80 | 0 | ◯ | ◯ |
| Working Example 70 | 10 | 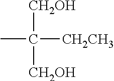 | H | H | CH₃(CH₂)₈CH((CH₂)₆CH₃)COOH | 1:1 | 10 | 10 | 80 | 0 | ◯ | ◯ |
| Working Example 71 | 11 | 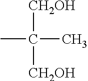 | H | H | CH₃(CH₂)₈CH((CH₂)₆CH₃)COOH | 1:1 | 10 | 10 | 80 | 0 | ◯ | ◯ |
| Working Example 72 | 12 | 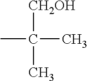 | H | H | CH₃(CH₂)₈CH((CH₂)₆CH₃)COOH | 1:1 | 10 | 10 | 80 | 0 | ◯ | ◯ |
| Working Example | 13 | (CH₂)₂OH | (CH₂)₂OH | (CH₂)₂OH | CH₃(CH₂)₈CH((CH₂)₆CH₃)COOH | 1:1 | 10 | 10 | 80 | 0 | ◯ | ◯ |
| 73 | | | | | | | | | | | | |
| Working Example | 14 | (CH₂)₂OH | H | H | CH₃(CH₂)₈CH((CH₂)₆CH₃)COOH | 1:1 | 10 | 10 | 80 | 0 | ◯ | ◯ |
| 74 | | | | | | | | | | | | |

| | | | | | | | | |
|---|---|---|---|---|---|---|---|---|
| Comparative | 25 | Sorbitan sesquiisostearate | 10 | 10 | 80 | 0 | ◯ | X |
| example | | | | | | | | |
| 39 | | | | | | | | |
| Comparative | 26 | Span 80 | 10 | 10 | 80 | 0 | ◯ | X |
| example | | | | | | | | |
| 40 | | | | | | | | |

### 6. Evaluation 2 of Compostions in Parallel Use of Surfactant and Additive for Imparting Interfacial Activity

The compositions of the working examples 75 to 88 and the comparative examples 41 to 44 were prepared. The components (A) and (B) of the present invention and a solvent were mixed with each other at the composition ratios as listed in Tables 13 and 14, and the additive, having the solvent distilled away, an oil agent and a surfactant were mixed at 80° C. and stirred, after which water was added thereinto and heated for 5 minutes while stirring the same to obtain an emulsion composition. It was determined in accordance with the dye method that the emulsified states of the resultant compositions were of W/O type.

A similar emulsion composition was also obtained by adding the components (A) and (B) as the additives of the present invention in a separate manner into an oil agent and a surfactant such that the composition ratios thereof were as listed in Tables 13 and 14, mixing and stirring them at 80° C., after which water was added thereinto and heated for 5 minutes while stirring the same.

As shown from the results in Tables 13 and 14, the working examples 75 to 88 of the present invention resulted in emulsion compositions which were found to have emulsion stabilities that were superior to those of the comparative examples 41 to 44. This result suggests that the additive containing the components (A) and (B) of the present invention exhibits an enhanced interfacial activity (emulsification) effect even in a W/O type emulsified state.

TABLE 13

| | | | | | | | Emulsion composition | | | | Emulsified | Emulsion |
|---|---|---|---|---|---|---|---|---|---|---|---|---|
| | Additive | | | | | | Composition ratio (wt. %) | | | | state | stability |
| | No. | 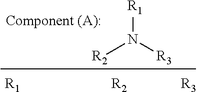 | | | Component (B) | Compounding molar ratio (A):(B) | Additive | Water | Oil agent Pertrolatum | Surfactant Sorbitan sesqiisostearate | Appearance | Evaluation 1 Centrifugation |
| Working Example 75 | 1 | 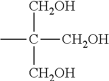 | H | H | CH₃(CH₂)₇CH═CH(CH₂)₇COOH | 1:1 | 10 | 10 | 78 | 2 | ◯ | ◯ |
| Working Example 76 | 2 | 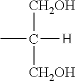 | H | H | CH₃(CH₂)₇CH═CH(CH₂)₇COOH | 1:1 | 10 | 10 | 78 | 2 | ◯ | ◯ |
| Working Example 77 | 3 | 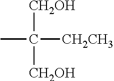 | H | H | CH₃(CH₂)₇CH═CH(CH₂)₇COOH | 1:1 | 10 | 10 | 78 | 2 | ◯ | ◯ |
| Working Example 78 | 4 | 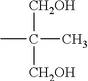 | H | H | CH₃(CH₂)₇CH═CH(CH₂)₇COOH | 1:1 | 10 | 10 | 78 | 2 | ◯ | ◯ |
| Working Example 79 | 5 | 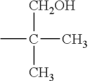 | H | H | CH₃(CH₂)₇CH═CH(CH₂)₇COOH | 1:1 | 10 | 10 | 78 | 2 | ◯ | ◯ |
| Working example | 6 | (CH₂)₂OH | (CH₂)₂OH | (CH₂)₂OH | CH₃(CH₂)₇CH═CH(CH₂)₇COOH | 1:1 | 10 | 10 | 78 | 2 | ◯ | ◯ |
| 80 | | | | | | | | | | | | |
| Working example | 7 | (CH₂)₂OH | H | H | CH₃(CH₂)₇CH═CH(CH₂)₇COOH | 1:1 | 10 | 10 | 78 | 2 | ◯ | ◯ |
| 81 | | | | | | | | | | | | |

| | | | | | | | | |
|---|---|---|---|---|---|---|---|---|
| Comparative | 25 | Sorbitan sesquiisostearate | 10 | 10 | 78 | 2 | ◯ | X |
| example | | | | | | | | |
| 41 | | | | | | | | |
| Comparative | 26 | Span 80 | 10 | 10 | 78 | 2 | ◯ | X |
| example | | | | | | | | |
| 42 | | | | | | | | |

TABLE 14

| | | | | | | | Emulsion composition | | | | Emulsified | Emulsion |
|---|---|---|---|---|---|---|---|---|---|---|---|---|
| | Additive | | | | | | Composition ratio (wt. %) | | | | state | stability |
| | No. | 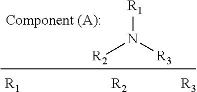 | | | Component (B) | Compounding molar ratio (A):(B) | Additive | Water | Oil agent Cetyl ethylhexa- noate | Surfactant Sorbitan sesqiisostearate | Appearance | Evaluation 1 Centrifugation |
| Working example 82 | 8 | 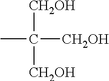 | H | H | CH₃(CH₂)₈CH((CH₂)₆CH₃)COOH | 1:1 | 10 | 10 | 78 | 2 | ◯ | ◯ |
| Working example 83 | 9 | 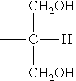 | H | H | CH₃(CH₂)₈CH((CH₂)₆CH₃)COOH | 1:1 | 10 | 10 | 78 | 2 | ◯ | ◯ |
| Working example 84 | 10 | 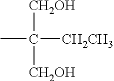 | H | H | CH₃(CH₂)₈CH((CH₂)₆CH₃)COOH | 1:1 | 10 | 10 | 78 | 2 | ◯ | ◯ |
| Working example 85 | 11 | 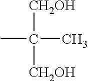 | H | H | CH₃(CH₂)₈CH((CH₂)₆CH₃)COOH | 1:1 | 10 | 10 | 78 | 2 | ◯ | ◯ |
| Working example 86 | 12 | 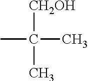 | H | H | CH₃(CH₂)₈CH((CH₂)₆CH₃)COOH | 1:1 | 10 | 10 | 78 | 2 | ◯ | ⊚ |
| Working example | 13 | (CH₂)₂OH | (CH₂)₂OH | (CH₂)₂OH | CH₃(CH₂)₈CH((CH₂)₆CH₃)COOH | 1:1 | 10 | 10 | 78 | 2 | ◯ | ⊚ |
| 87 | | | | | | | | | | | | |
| Working example | 11 | (CH₂)₂OH | H | H | CH₃(CH₂)₈CH((CH₂)₆CH₃)COOH | 1:1 | 10 | 10 | 78 | 2 | ◯ | ◯ |
| 85 | | | | | | | | | | | | |

| | | | | | | | | |
|---|---|---|---|---|---|---|---|---|
| Comparative | 23 | Sorbitan sesquiisostearate | 10 | 10 | 78 | 2 | ◯ | X |
| example | | | | | | | | |
| 43 | | | | | | | | |
| Comparative | 26 | Span 80 | 10 | 10 | 78 | 2 | ◯ | X |
| example | | | | | | | | |
| 44 | | | | | | | | |

### 7. Feeling of Use 2 of Composition Using Additive for Imparting Interfacial Activity

The compositions of the working examples 89 to 102 and the comparative examples 45 to 48, having composition ratios as listed in Tables 15 and 16 and prepared by the prescription similar to those in the section 5, were evaluated with respect to the feelings of use using the same criteria as those of the section 3.

As can be seen from the results shown in Tables 15 and 16, the working examples 89 to 102 of the present invention were superior in terms of feeling of use compared to the comparative examples 45 to 48 since these working examples had evaluations of O or greater with respect to the spreadability, non-stickiness and skin compatibility, which shows that the additives containing the components (A) and (B) of the present invention are excellent in feeling of use even in a W/O type emulsified state.

TABLE 15

| | | | | | | | Emulsion composition | | | | Feeling of | | |
|---|---|---|---|---|---|---|---|---|---|---|---|---|---|
| | Additive | | | | | | Composition ratio (wt. %) | | | | use | | |
| | No. | 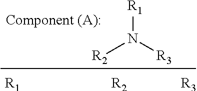 | | | Component (B) | Compounding molar ratio (A):(B) | Additive | Water | Oil agent Pertrolatum | Surfactant Sorbitan sesqiisostearate | Stretch ability | non- stickiness | Skin compatibility |
| Working example 89 | 1 | 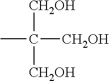 | H | H | CH₃(CH₂)₇CH═CH(CH₂)₇COOH | 1:1 | 10 | 10 | 80 | 0 | ◯ | ◯ | ◯ |
| Working example 90 | 2 | 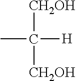 | H | H | CH₃(CH₂)₇CH═CH(CH₂)₇COOH | 1:1 | 10 | 10 | 80 | 0 | ◯ | ◯ | ◯ |
| Working example 91 | 3 | 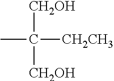 | H | H | CH₃(CH₂)₇CH═CH(CH₂)₇COOH | 1:1 | 10 | 10 | 80 | 0 | ◯ | ◯ | ◯ |
| Working example 92 | 4 | 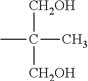 | H | H | CH₃(CH₂)₇CH═CH(CH₂)₇COOH | 1:1 | 10 | 10 | 80 | 0 | ◯ | ◯ | ◯ |
| Working example 93 | 5 | 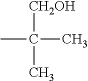 | H | H | CH₃(CH₂)₇CH═CH(CH₂)₇COOH | 1:1 | 10 | 10 | 80 | 0 | ◯ | ◯ | ◯ |
| Working example | 6 | (CH₂)₂OH | (CH₂)₂OH\| | (CH₂)₂OH | CH₃(CH₂)₇CH═CH(CH₂)₇COOH | 1:1 | 10 | 10 | 80 | 0 | ◯ | ◯ | ◯ |
| 94 | | | | | | | | | | | | | |
| Working example | 7 | (CH₂)₂OH | H | H | CH₃(CH₂)₇CH═CH(CH₂)₇COOH | 1:1 | 10 | 10 | 80 | 0 | ◯ | ◯ | ◯ |
| 95 | | | | | | | | | | | | | |

| | | | | | | | | | |
|---|---|---|---|---|---|---|---|---|---|
| Comparative | 25 | Sorbitan sesquiisostearate | 10 | 10 | 80 | 0 | Δ | Δ | Δ |
| example | | | | | | | | | |
| 45 | | | | | | | | | |
| Comparative | 26 | Span 80 | 10 | 10 | 80 | 0 | Δ | Δ | Δ |
| example | | | | | | | | | |
| 46 | | | | | | | | | |

TABLE 16

| | | |
|---|---|---|
| | Emulsion composition | Feeling of |
| Additive | Composition ratio (wt. %) | use |

| | No. | 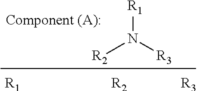 | | | Component (B) | Com- pounding molar ratio (A):(B) | Additive | Water | Oil agent Cetyl ethylhexa- noate | Surfactant Sorbitan sesqiisostearate | Stretch ability | non- stickiness | Skin compatibility |
|---|---|---|---|---|---|---|---|---|---|---|---|---|---|
| Working example 96 | 8 | 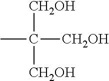 | H | H | CH₃(CH₂)₈CH((CH₂)₆CH₃)COOH | 1:1 | 10 | 10 | 80 | 0 | ◯ | ◯ | ◯ |
| Working example 97 | 9 | 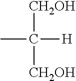 | H | H | CH₃(CH₂)₈CH((CH₂)₆CH₃)COOH | 1:1 | 10 | 10 | 80 | 0 | ◯ | ◯ | ◯ |
| Working example 98 | 10 | 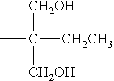 | H | H | CH₃(CH₂)₈CH((CH₂)₆CH₃)COOH | 1:1 | 10 | 10 | 80 | 0 | ◯ | ◯ | ◯ |
| Working example 99 | 11 | 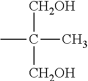 | H | H | CH₃(CH₂)₈CH((CH₂)₆CH₃)COOH | 1:1 | 10 | 10 | 80 | 0 | ◯ | ◯ | ◯ |
| Working example 100 | 12 | 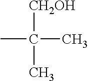 | H | H | CH₃(CH₂)₈CH((CH₂)₆CH₃)COOH | 1:1 | 10 | 10 | 80 | 0 | ◯ | ◯ | ◯ |
| Working example | 13 | (CH₂)₂OH | (CH₂)₂OH | (CH₂)₂OH | CH₃(CH₂)₈CH((CH₂)₆CH₃)COOH | 1:1 | 10 | 10 | 80 | 0 | ◯ | ◯ | ◯ |
| 101 | | | | | | | | | | | | | |
| Working example | 14 | (CH₂)₂OH | H | H | CH₃(CH₂)₈CH((CH₂)₆CH₃)COOH | 1:1 | 10 | 10 | 80 | 0 | ◯ | ◯ | ◯ |
| 102 | | | | | | | | | | | | | |

| | | | | | | | | | |
|---|---|---|---|---|---|---|---|---|---|
| Comparative | 25 | Sorbitan sesquiisostearate | 10 | 10 | 80 | 0 | Δ | Δ | Δ |
| example | | | | | | | | | |
| 47 | | | | | | | | | |
| Comparative | 26 | Span 80 | 10 | 10 | 80 | 0 | Δ | Δ | Δ |
| example | | | | | | | | | |
| 48 | | | | | | | | | |

### 8. Feeling of Use 2 of Composition in Parallel Use of Surfactant and Additive for Imparting Interfacial Activity

The compositions of the working examples 103 to 116 and the comparative examples 49 to 52, having composition ratios as listed in Tables 17 and 18 and prepared by the prescription similar to those in section 6, were evaluated with respect to the feelings of use using the same criteria as those of the section 3.

As can be seen from the results shown in Tables 17 and 18, the working examples 103 to 116 of the present invention had evaluations of O or greater with respect to the spreadability, non-stickiness and skin compatibility, which therefore shows the superiority of the additives containing the components (A) and (B) of the present invention compared to the comparative examples 49 to 52.

TABLE 17

| | | | | | | | Emulsion composition | | | | Feeling of | | |
|---|---|---|---|---|---|---|---|---|---|---|---|---|---|
| | Additive | | | | | | Composition ratio (wt. %) | | | | use | | |
| | No. | 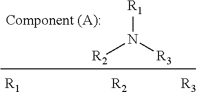 | | | Component (B) | Com- pounding molar ratio (A):(B) | Additive | Water | Oil agent Pertrolatum | Surfactant Sorbitan sesqiisostearate | Stretch ability | non- stickiness | Skin compatibility |
| Working example 103 | 1 | 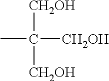 | H | H | CH₃(CH₂)₇CH═CH(CH₂)₇COOH | 1:1 | 10 | 10 | 78 | 2 | ◯ | ◯ | ◯ |
| Working example 104 | 2 | 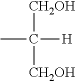 | H | H | CH₃(CH₂)₇CH═CH(CH₂)₇COOH | 1:1 | 10 | 10 | 78 | 2 | ◯ | ◯ | ◯ |
| Working example 105 | 3 | 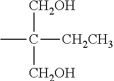 | H | H | CH₃(CH₂)₇CH═CH(CH₂)₇COOH | 1:1 | 10 | 10 | 78 | 2 | ◯ | ◯ | ◯ |
| Working example 106 | 4 | 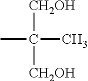 | H | H | CH₃(CH₂)₇CH═CH(CH₂)₇COOH | 1:1 | 10 | 10 | 78 | 2 | ◯ | ◯ | ◯ |
| Working example 107 | 5 | 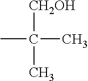 | H | H | CH₃(CH₂)₇CH═CH(CH₂)₇COOH | 1:1 | 10 | 10 | 78 | 2 | ◯ | ◯ | ◯ |
| Working example | 6 | (CH₂)₂OH | (CH₂)₂OH | (CH₂)₂OH | CH₃(CH₂)₇CH═CH(CH₂)₇COOH | 1:1 | 10 | 10 | 78 | 2 | ◯ | ◯ | ◯ |
| 108 | | | | | | | | | | | | | |
| Working example | 7 | (CH₂)₂OH | H | H | CH₃(CH₂)₇CH═CH(CH₂)₇COOH | 1:1 | 10 | 10 | 78 | 2 | ◯ | ◯ | ◯ |
| 109 | | | | | | | | | | | | | |

| | | | | | | | | | |
|---|---|---|---|---|---|---|---|---|---|
| Comparative | 25 | Sorbitan sesquiisostearate | 10 | 10 | 78 | 2 | Δ | Δ | Δ |
| example | | | | | | | | | |
| 49 | | | | | | | | | |
| Comparative | 26 | Span 80 | 10 | 10 | 78 | 2 | Δ | Δ | Δ |
| example | | | | | | | | | |
| 50 | | | | | | | | | |

TABLE 18

| | | | | | | | Emulsion composition | | | | Feeling of | | |
|---|---|---|---|---|---|---|---|---|---|---|---|---|---|
| | Additive | | | | | | Composition ratio (wt. %) | | | | use | | |
| | No. | 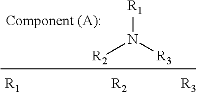 | | | Component (B) | Com- pounding molar ratio (A):(B) | Additive | Water | Oil agent Cetyl ethylhexa- noate | Surfactant Sorbitan sesqiisostearate | Stretch ability | non- stickiness | Skin compatibility |
| Working example 110 | 8 | 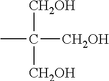 | H | H | CH₃(CH₂)₈CH((CH₂)₆CH₃)COOH | 1:1 | 10 | 10 | 78 | 2 | ◯ | ◯ | ◯ |
| Working example 111 | 9 | 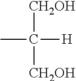 | H | H | CH₃(CH₂)₈CH((CH₂)₆CH₃)COOH | 1:1 | 10 | 10 | 78 | 2 | ◯ | ◯ | ◯ |
| Working example 112 | 10 | 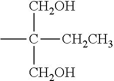 | H | H | CH₃(CH₂)₈CH((CH₂)₆CH₃)COOH | 1:1 | 10 | 10 | 78 | 2 | ◯ | ◯ | ◯ |
| Working example 113 | 11 | 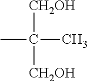 | H | H | CH₃(CH₂)₈CH((CH₂)₆CH₃)COOH | 1:1 | 10 | 10 | 78 | 2 | ◯ | ◯ | ◯ |
| Working example 114 | 12 | 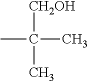 | H | H | CH₃(CH₂)₈CH((CH₂)₆CH₃)COOH | 1:1 | 10 | 10 | 78 | 2 | ◯ | ◯ | ◯ |
| Working example | 13 | (CH₂)₂OH | (CH₂)₂OH | (CH₂)₂OH | CH₃(CH₂)₈CH((CH₂)₆CH₃)COOH | 1:1 | 10 | 10 | 78 | 2 | — | — | — |
| 115 | | | | | | | | | | | | | |
| Working example | 14 | (CH₂)₂OH | H | H | CH₃(CH₂)₈CH((CH₂)₆CH₃)COOH | 1:1 | 10 | 10 | 78 | 2 | — | — | — |
| 116 | | | | | | | | | | | | | |

| | | | | | | | | | |
|---|---|---|---|---|---|---|---|---|---|
| Comparative | 25 | Sorbitan sesquiisostearate | 10 | 10 | 78 | 2 | Δ | Δ | Δ |
| example | | | | | | | | | |
| 51 | | | | | | | | | |
| Comparative | 26 | Span 80 | 10 | 10 | 78 | 2 | Δ | Δ | Δ |
| example | | | | | | | | | |
| 52 | | | | | | | | | |

### 9. Composition Evaluation and Lubricity Evaluation of Composition Using Additive for Imparting Interfacial Activity.

### 9-1 Composition Evaluation

The compositions of the working examples 117 to 124 and 141 to 148 and the comparative examples 53 and 55 to 58 were prepared. The components (A) and (B) of the present invention and a solvent were mixed with each other at the composition ratios (1) to (3) as listed in Tables 19A, 19B, 19C, 20A, 20B and 20C, and the additives, having the solvent distilled away, water and an oil agent were mixed with each other, and stirred for three hours at room temperature to obtain an emulsion composition.

A similar emulsion composition was also obtained by mixing the components (A) and (B) as the additives of the present invention in a separate manner into an oil agent and water such that the composition ratios thereof were as listed in Tables 19A, 19B, 20A and 20B, and stirring them at room temperature for three hours.

The resultant emulsion compositions had a low electric conductivity as a result of electric conductance measurement, and the continuous phase was an oil phase, which lead to the conclusion that the resultant emulsion compositions were of W/O type emulsion.

As for the stability of the resultant emulsion composition, the obtained compositions were left to stand still in a thermostatic bath controlled at 25° C. to evaluate a change in the emulsified state over time based on the following criteria.

### <Evaluation Criteria>

⊚: No phase separation was observed for 30 days.
O: Phase separation was observed within 14 days.
x: Phase separation was observed within 7 days.

As shown from the results in Tables 19A, 19B, 19C, 20A, 20B and 21C, the working examples 117 to 124 and 141 to 148 resulted in emulsion compositions with reference to the comparative examples 53 and 55 to 58, and it was found that these working examples were superior in terms of emulsion stability compared to the comparative examples 53 and 55 to 58. This result suggests that the additive containing the components (A) and (B) of the present invention exhibits an enhanced interfacial activity (emulsification) effect.

Among the working examples, the examples 117 and 121 having three hydroxy groups in the branched hydroxyaliphatic hydrocarbon group in the component (A) had an emulsion stability which is superior to the working examples 118, 119, 122, 123, 141 and 145 having two hydroxy groups and to the working examples 142 and 146 having one hydroxy group. This result indicated that the hydroxy groups in the branched hydroxyaliphatic hydrocarbon group in the component (A) contributed to the interfacial activity (emulsification) effect, suggesting that those having three or more hydroxy groups are preferable.

### 9-2. Lubricity Evaluation

Dynamic friction coefficients of the composition of the working examples 117 to 124 and 141 to 148 and the comparative examples 53 and 55 to 58 as prepared in accordance with the section 9-1 were measured using pendulum type oiliness friction tester (KOBELCO MACHINERY ENGINEERING Co., Ltd.).

As shown from the results in Tables 19A, 19B, 19C, 20A, 20B and 21C, the respective emulsion compositions (at the composition ratios (1) to (3)) of the working examples 117 to 124 and 141 to 148 had smaller dynamic friction coefficients compared to those of the comparative examples 53 and 55 to 58. Accordingly, it is to be suggested that the additives containing the components (A) and (B) according to the present invention contributed to the formation of highly-stable emulsion compositions, which causes the emulsion compositions to have an enhanced affinity to the target friction material to thereby exhibit an enhanced lubrication effect.

Further, among the components (A), the working examples 117 to 119, 141, 142 had dynamic friction coefficients that were smaller than those of the working examples 120, 143, 144 and that the working examples 121 to 123, 145 and 146 had dynamic friction coefficients that were smaller than those of the working examples 124, 147 and 148, which shows that the additives having a branched hydroxyaliphatic hydrocarbon group in the component (A) have enhanced lubrication effects. Furthermore, dynamic friction coefficients of the working examples 117 and 121 were relatively small among them, which suggests that those having three or more hydroxy groups in a branched hydroxyaliphatic hydrocarbon group are preferable.

Moreover, among compounds where the component (A) is a compound having three or more hydroxy groups in a branched hydroxyaliphatic hydrocarbon group, the working examples 117 and 121 in which the component (B) has an unsaturated fatty acid having 8 to 22 carbon atoms or a branched fatty acid having 8 to 22 carbon atoms had smaller dynamic friction coefficients compared to those of the comparative example 55 having a linear fatty acid having 8 to 22 carbon atoms. It was therefore found that the additives having an unsaturated fatty acid having 8 to 22 carbon atoms or a branched fatty acid having 8 to 22 carbon atoms in the component (B) have enhanced lubrication effects.

Examples 117 to 124 and 141 to 148 containing a hydroxyaliphatic hydrocarbon group in the component (A) had smaller dynamic friction coefficients compared to those of the comparative example 56 and 58 having no hydroxyaliphatic hydrocarbon group in the component (A). It was found from these results that the additives containing a hydroxyaliphatic hydrocarbon group in the component (A) have enhanced lubrication effects.

TABLE 19A

| | Additive | | | | | |
|---|---|---|---|---|---|---|
| | No. | 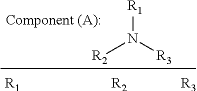 | | | Component (B) | Compounding molar ratio (A):(B) |
| Working example 117 | 1 | 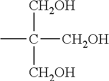 | H | H | CH₃(CH₂)₇CH═CH(CH₂)₇COOH | 1:1 |
| Working example 118 | 2 | 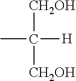 | H | H | CH₃(CH₂)₇CH═CH(CH₂)₇COOH | 1:1 |
| Working example 119 | 3 | 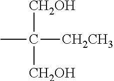 | H | H | CH₃(CH₂)₇CH═CH(CH₂)₇COOH | 1:1 |
| Working example 141 | 4 | 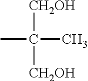 | H | H | CH₃(CH₂)₇CH═CH(CH₂)₇COOH | 1:1 |
| Working example 142 | 5 | 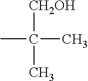 | H | H | CH₃(CH₂)₇CH═CH(CH₂)₇COOH | 1:1 |
| Working example | 6 | (CH₂)₂OH | (CH₂)₂OH | (CH₂)₂OH | CH₃(CH₂)₇CH═CH(CH₂)₇COOH | 1:1 |
| 120 | | | | | | |
| Working example | 28 | (CH₂)₂OH | (CH₂)₂OH | H | CH₃(CH₂)₇CH═CH(CH₂)₇COOH | 1:1 |
| 143 | | | | | | |
| Working example | 7 | (CH₂)₂OH | H | H | CH₃(CH₂)₇CH═CH(CH₂)₇COOH | 1:1 |
| 144 | | | | | | |
| Comparative example 55 | 16 | 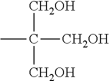 | H | H | CH₃(CH₂)₁₆COOH | 1:1 |
| Comparative | 29 | CH₂CH₃ | CH₂CH₃ | CH₂CH₃ | CH₃(CH₂)₇CH═CH(CH₂)₇COOH | 1:1 |
| example | | | | | | |
| 56 | | | | | | |

| | | |
|---|---|---|
| Comparative | 27 | E-451D (Polyether-based non-ionic surfactant) |
| example | | |
| 53 | | |
| Comparative | 30 | Glycerin fatty acid ester |
| example | | |
| 57 | | |

TABLE 19B

| | Emulsion composition | | | | Emulsion composition | | | | Emulsion composition | | | |
|---|---|---|---|---|---|---|---|---|---|---|---|---|
| | Composition ratio (1) (wt. %) | | | | Composition ratio (2) (wt. %) | | | | Composition ratio (3) (wt. %) | | | |
| | | | Oil | | | | Oil | | | | Oil | |
| | | | agent | | | | agent | | | | agent | |
| | | | OTMP- | Surfactant | | | OTMP- | Surfactant | | | OTMP- | Surfactant |
| | Additive | Water | 300 | E-451D | Additive | Water | 300 | E-451D | Additive | Water | 300 | E-451D |
| Working | 10 | 30 | 60 | 0 | 10 | 50 | 40 | 0 | 10 | 70 | 20 | 0 |
| example 117 | | | | | | | | | | | | |
| Working | 10 | 30 | 60 | 0 | 10 | 50 | 40 | 0 | 10 | 70 | 20 | 0 |
| example 118 | | | | | | | | | | | | |
| Working | 10 | 30 | 60 | 0 | 10 | 50 | 40 | 0 | 10 | 70 | 20 | 0 |
| example 119 | | | | | | | | | | | | |
| Working | 10 | 30 | 60 | 0 | 10 | 50 | 40 | 0 | 10 | 70 | 20 | 0 |
| example 141 | | | | | | | | | | | | |
| Working | 10 | 30 | 60 | 0 | 10 | 50 | 40 | 0 | 10 | 70 | 20 | 0 |
| example 142 | | | | | | | | | | | | |
| Working | 10 | 30 | 60 | 0 | 10 | 50 | 40 | 0 | 10 | 70 | 20 | 0 |
| example 120 | | | | | | | | | | | | |
| Working | 10 | 30 | 60 | 0 | 10 | 50 | 40 | 0 | 10 | 70 | 20 | 0 |
| example 143 | | | | | | | | | | | | |
| Working | 10 | 30 | 60 | 0 | 10 | 50 | 40 | 0 | 10 | 70 | 20 | 0 |
| example 144 | | | | | | | | | | | | |
| Comparative | 10 | 30 | 60 | 0 | 10 | 50 | 40 | 0 | 10 | 70 | 20 | 0 |
| example 55 | | | | | | | | | | | | |
| Comparative | 10 | 30 | 60 | 0 | 10 | 50 | 40 | 0 | 10 | 70 | 20 | 0 |
| example 56 | | | | | | | | | | | | |
| Comparative | 10 | 30 | 60 | 0 | 10 | 50 | 40 | 0 | 10 | 70 | 20 | 0 |
| example 53 | | | | | | | | | | | | |
| Comparative | 10 | 30 | 60 | 0 | 10 | 50 | 40 | 0 | 10 | 70 | 20 | 0 |
| example 57 | | | | | | | | | | | | |

TABLE 19C

| | Emulsion stability (at 25° C.) | | | Dynamic friction coefficient | | |
|---|---|---|---|---|---|---|
| | Emulsion | Emulsion | Emulsion | Emulsion | Emulsion | Emulsion |
| | ratio (1) | ratio (2) | ratio (3) | ratio (1) | ratio (2) | ratio (3) |
| | composition | composition | composition | composition | composition | composition |
| | Composition | Composition | Composition | Composition | Composition | Composition |
| Working | ⊚ | ⊚ | ⊚ | 0.113 | 0.119 | 0.123 |
| example 117 | | | | | | |
| Working | ⊚ | ◯ | ◯ | 0.114 | 0.121 | 0.124 |
| example 118 | | | | | | |
| Working | ⊚ | ◯ | ◯ | 0.115 | 0.122 | 0.128 |
| example 119 | | | | | | |
| Working | ⊚ | ◯ | ◯ | 0.115 | 0.122 | 0.129 |
| example 141 | | | | | | |
| Working | ⊚ | ◯ | ◯ | 0.118 | 0.125 | 0.129 |
| example 142 | | | | | | |
| Working | ⊚ | ◯ | ◯ | 0.119 | 0.130 | 0.129 |
| example 120 | | | | | | |
| Working | ⊚ | ◯ | ◯ | 0.122 | 0.133 | 0.135 |
| example 143 | | | | | | |
| Working | ⊚ | ◯ | ◯ | 0.125 | 0.135 | 0.138 |
| example 144 | | | | | | |
| Comparative | ◯ | X | X | 0.128 | 0.140 | 0.145 |
| example 55 | | | | | | |
| Comparative | ◯ | X | X | 0.128 | 0.140 | 0.145 |
| example 56 | | | | | | |
| Comparative | X | x | X | 0.130 | 0.142 | 0.153 |
| example 53 | | | | | | |
| Comparative | X | X | X | 0.135 | 0.145 | 0.158 |
| example 57 | | | | | | |

TABLE 20A

| | Additive | | | | | |
|---|---|---|---|---|---|---|
| | No. | 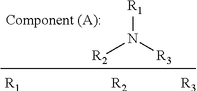 | | | Component (B) | Compounding molar ratio (A):(B) |
| Working example 121 | 8 | 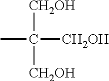 | H | H | CH₃(CH₂)₈CH((CH₂)₆CH₃)COOH | 1:1 |
| Working example 122 | 9 | 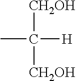 | H | H | CH₃(CH₂)₈CH((CH₂)₆CH₃)COOH | 1:1 |
| Working example 123 | 10 | 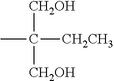 | H | H | CH₃(CH₂)₈CH((CH₂)₆CH₃)COOH | 1:1 |
| Working example 145 | 11 | 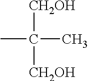 | H | H | CH₃(CH₂)₈CH((CH₂)₆CH₃)COOH | 1:1 |
| Working example 146 | 12 | 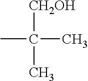 | H | H | CH₃(CH₂)₈CH((CH₂)₆CH₃)COOH | 1:1 |
| Working example | 13 | (CH₂)₂OH | (CH₂)₂OH | (CH₂)₂OH | CH₃(CH₂)₈CH((CH₂)₆CH₃)COOH | 1:1 |
| 124 | | | | | | |
| Working example | 31 | (CH₂)₂OH | (CH₂)₂OH | H | CH₃(CH₂)₈CH((CH₂)₆CH₃)COOH | 1:1 |
| 147 | | | | | | |
| Working example | 14 | (CH₂)₂OH | H | H | CH₃(CH₂)₈CH((CH₂)₆CH₃)COOH | 1:1 |
| 148 | | | | | | |
| Comparative example 55 | 16 | 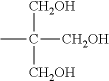 | H | H | CH₃(CH₂)₁₆COOH | 1:1 |
| Comparative | 32 | CH₂CH₃ | CH₂CH₃ | CH₂CH₃ | CH₃(CH₂)₈CH((CH₂)₆CH₃)COOH | 1:1 |
| example | | | | | | |
| 58 | | | | | | |

| Comparative | 27 | E-451D (Polyether-based non-ionic surfactant) |
|---|---|---|
| example | | |
| 53 | | |
| Comparative | 30 | Glycerin fatty acid ester |
| example | | |
| 57 | | |

TABLE 20B

| | Emulsion composition | | | | Emulsion composition | | | | Emulsion composition | | | |
|---|---|---|---|---|---|---|---|---|---|---|---|---|
| | Composition ratio (1) (wt. %) | | | | Composition ratio (2) (wt. %) | | | | Composition ratio (3) (wt. %) | | | |
| | | | Oil | | | | Oil | | | | Oil | |
| | | | agent | | | | agent | | | | agent | |
| | | | OTMP- | Surfactant | | | OTMP- | Surfactant | | | OTMP- | Surfactant |
| | Additive | Water | 300 | E-451D | Additive | Water | 300 | E-451D | Additive | Water | 300 | E-451D |
| Working | 10 | 30 | 60 | 0 | 10 | 50 | 40 | 0 | 10 | 70 | 20 | 0 |
| example 121 | | | | | | | | | | | | |
| Working | 10 | 30 | 60 | 0 | 10 | 50 | 40 | 0 | 10 | 70 | 20 | 0 |
| example 122 | | | | | | | | | | | | |
| Working | 10 | 30 | 60 | 0 | 10 | 50 | 40 | 0 | 10 | 70 | 20 | 0 |
| example 123 | | | | | | | | | | | | |
| Working | 10 | 30 | 60 | 0 | 10 | 50 | 40 | 0 | 10 | 70 | 20 | 0 |
| example 145 | | | | | | | | | | | | |
| Working | 10 | 30 | 60 | 0 | 10 | 50 | 40 | 0 | 10 | 70 | 20 | 0 |
| example 146 | | | | | | | | | | | | |
| Working | 10 | 30 | 60 | 0 | 10 | 50 | 40 | 0 | 10 | 70 | 20 | 0 |
| example 124 | | | | | | | | | | | | |
| Working | 10 | 30 | 60 | 0 | 10 | 50 | 40 | 0 | 10 | 70 | 20 | 0 |
| example 147 | | | | | | | | | | | | |
| Working | 10 | 30 | 60 | 0 | 10 | 50 | 40 | 0 | 10 | 70 | 20 | 0 |
| example 148 | | | | | | | | | | | | |
| Comparative | 10 | 30 | 60 | 0 | 10 | 50 | 40 | 0 | 10 | 70 | 20 | 0 |
| example 55 | | | | | | | | | | | | |
| Comparative | 10 | 30 | 60 | 0 | 10 | 50 | 40 | 0 | 10 | 70 | 20 | 0 |
| example 58 | | | | | | | | | | | | |
| Comparative | 10 | 30 | 60 | 0 | 10 | 50 | 40 | 0 | 10 | 70 | 20 | 0 |
| example 53 | | | | | | | | | | | | |
| Comparative | 10 | 30 | 60 | 0 | 10 | 50 | 40 | 0 | 10 | 70 | 20 | 0 |
| example 57 | | | | | | | | | | | | |

TABLE 20C

| | Emulsion stability (at 25° C.) | | | Dynamic friction coefficient | | |
|---|---|---|---|---|---|---|
| | Emulsion | Emulsion | Emulsion | Emulsion | Emulsion | Emulsion |
| | ratio (1) | ratio (2) | ratio (3) | ratio (1) | ratio (2) | ratio (3) |
| | composition | composition | composition | composition | composition | composition |
| | Composition | Composition | Composition | Composition | Composition | Composition |
| Working | ⊚ | ⊚ | ⊚ | 0.115 | 0.117 | 0.130 |
| example 121 | | | | | | |
| Working | ⊚ | ◯ | ◯ | 0.116 | 0.122 | 0.131 |
| example 122 | | | | | | |
| Working | ⊚ | ◯ | ◯ | 0.119 | 0.127 | 0.132 |
| example 123 | | | | | | |
| Working | ⊚ | ◯ | ◯ | 0.119 | 0.127 | 0.132 |
| example 145 | | | | | | |
| Working | ⊚ | ◯ | ◯ | 0.120 | 0.128 | 0.133 |
| example 146 | | | | | | |
| Working | ⊚ | ◯ | ◯ | 0.122 | 0.139 | 0.134 |
| example 124 | | | | | | |
| Working | ⊚ | ◯ | ◯ | 0.123 | 0.139 | 0.138 |
| example 147 | | | | | | |
| Working | ⊚ | ◯ | ◯ | 0.124 | 0.139 | 0.140 |
| example 148 | | | | | | |
| Comparative | ◯ | X | X | 0.128 | 0.140 | 0.145 |
| example 55 | | | | | | |
| Comparative | ◯ | X | X | 0.128 | 0.140 | 0.145 |
| example 58 | | | | | | |
| Comparative | X | X | X | 0.130 | 0.142 | 0.153 |
| example 53 | | | | | | |
| Comparative | X | X | X | 0.135 | 0.145 | 0.158 |
| example 57 | | | | | | |

### 10. Composition Evaluation and Lubricity Evaluation 1 of Composition in Parallel Use of Surfactant and Additive for Imparting Interfacial Activity.

### 10-1. Composition Evaluation

The compositions of the working examples 125 to 132 and the comparative example 54 were prepared. The components (A) and (B) of the present invention and a solvent were mixed with each other at the emulsion composition ratios (1) to (3) as listed in Tables 21A, 21B, 22A and 22B, and the additives. having the solvent distilled away, water, an oil agent and a surfactant were mixed with each other and stirred for three hours at room temperature to obtain an emulsion composition.

A similar emulsion composition was also obtained by mixing the components (A) and (B) as the additives of the present invention in a separate manner into an oil agent and water such that the composition ratios thereof were as listed in Tables 21A, 21B, 22A and 22B, and stirring them at room temperature for three hours. The resultant emulsion compositions had a low electric conductivity as a result of electric conductance measurement, and the continuous phase was an oil phase, which lead to the conclusion that the resultant emulsion compositions were of W/O type emulsion.

Stabilities of the resultant emulsion compositions were evaluated on the basis of the same criteria of the section 9-1.

It was found from the results in Tables 21A, 21B, 22A and 22B that the working examples 125 to 132 of the present invention resulted in emulsion compositions with reference to those of the comparative example 54.

Further, as for the correlation between the structure of the components (A) and (B) and the interfacial activity (emulsification) effects, a tendency similar to the correlation between the structure of the components (A) and (B) and the interfacial activity (emulsification) effects as set forth in the section 9 was observed.

10-2. Lubricity Evaluation

Dynamic friction coefficients of the compositions of the working examples 125 to 132 and the comparative example 54 as prepared for the composition evaluation described above were measured using pendulum type oiliness friction tester (KOBELCO MACHINERY ENGINEERING Co., Ltd.).

As shown from the results in Tables 21A, 21B, 22A and 22B, the respective emulsion compositions (1) to (3) of the working examples 125 to 132 had smaller dynamic friction coefficients compared to that of the comparative example 54. Accordingly, it is to be suggested that the additives containing the components (A) and (B) according to the present invention contribute to the formations of the highly stable emulsion compositions, which causes the emulsion compositions to have an enhanced affinity to the friction target material to thereby exhibit an enhanced lubrication effect.

Further, as for the correlation between the structure of the components (A) and (B) and the dynamic friction coefficient, a tendency similar to the correlation between the structure of the components (A) and (B) and the interfacial activity (emulsification) effects as set forth in the section 9-2 was observed.

Meanwhile, when comparing the additives having the same components of (A) and (B) (between the working example 117 and the working example 125 or between the working example 121 and the working example 129) and further comparing the working examples 117 and 121 (10 wt. % of the additives of the present invention) with the working examples 125 and 129 (8 wt % of the additives of the present invention plus 2 wt. % of surfactant=10 wt. % in total), the working examples 117 and 121, having been emulsified with the additives of the present invention alone, had favorable emulsification stabilities and reduced dynamic friction coefficients, which therefore suggests that the additives of the present invention are excellent in emulsification capability (surfactant functionality) and lubricating efficacy.

TABLE 21A

| | | | | | | | Emulsion composition | | | | Emulsion composition | | | | Emulsion composition | | | |
|---|---|---|---|---|---|---|---|---|---|---|---|---|---|---|---|---|---|---|
| | Additive | | | | | | Composition ratio (1) (wt. %) | | | | Composition ratio (2) (wt. %) | | | | Composition ratio (3) (wt. %) | | | |
| | No. | 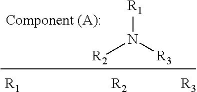 | | | Component (B) | Com- pound- ing molar ratio (A):(B) | Addi- tive | Water | Oil agent OTMP- 300 | Sur- factant E-451D | Addi- tive | Water | Oil agent OTMP- 300 | Sur- factant E-451D | Addi- tive | Water | Oil agent OTMP- 300 | Sur- factant E-451D |
| Work- ing ex- ample 125 | 1 | 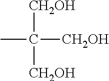 | H | H | CH₃(CH₂)₇CH═CH(CH₂)₇COOH | 1:1 | 8 | 30 | 60 | 2 | 8 | 50 | 40 | 2 | 8 | 70 | 20 | 2 |
| Work- ing ex- ample 126 | 2 | 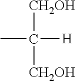 | H | H | CH₃(CH₂)₇CH═CH(CH₂)₇COOH | 1:1 | 8 | 30 | 60 | 2 | 8 | 50 | 40 | 2 | 8 | 70 | 20 | 2 |
| Work- ing ex- ample 127 | 3 | 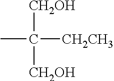 | H | H | CH₃(CH₂)₇CH═CH(CH₂)₇COOH | 1:1 | 8 | 30 | 60 | 2 | 8 | 50 | 40 | 2 | 8 | 70 | 20 | 2 |
| Work- | 6 | (CH₂)₂OH | (CH₂)₂OH | (CH₂)₂OH | CH₃(CH₂)₇CH═CH(CH₂)₇COOH | 1:1 | 8 | 30 | 60 | 2 | 8 | 50 | 40 | 2 | 8 | 70 | 20 | 2 |
| ing | | | | | | | | | | | | | | | | | | |
| ex- | | | | | | | | | | | | | | | | | | |
| ample | | | | | | | | | | | | | | | | | | |
| 128 | | | | | | | | | | | | | | | | | | |

| | | | | | | | | | | | | | | |
|---|---|---|---|---|---|---|---|---|---|---|---|---|---|---|
| Compar- | 27 | E-451D (Polyether-based non-ionic surfactant) | 8 | 30 | 60 | 2 | 8 | 50 | 40 | 2 | 8 | 70 | 20 | 2 |
| ative | | | | | | | | | | | | | | |
| ex- | | | | | | | | | | | | | | |
| ample | | | | | | | | | | | | | | |

54

TABLE 21B

| | Additive | | | | | | Stability (at 25° C.) | | | Dynamic friction coefficient | | |
|---|---|---|---|---|---|---|---|---|---|---|---|---|
| | No. | 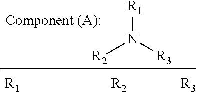 | | | Component (B) | Compounding molar ratio (A):(B) | Emulsion composition Composition ratio (1) (wt. %) | Emulsion composition Composition ratio (2) (wt. %) | Emulsion composition Composition ratio (3) (wt. %) | Emulsion composition Composition ratio (1) (wt. %) | Emulsion composition Composition ratio (2) (wt. %) | Emulsion composition Composition ratio (3) (wt. %) |
| Working example 125 | 1 | 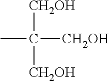 | H | H | CH₃(CH₂)₇CH═CH(CH₂)₇COOH | 1:1 | ⊚ | ⊚ | ⊚ | 0.117 | 0.122 | 0.124 |
| Working example 126 | 2 | 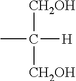 | H | H | CH₃(CH₂)₇CH═CH(CH₂)₇COOH | 1:1 | ⊚ | ◯ | ◯ | 0.116 | 0.123 | 0.128 |
| Working example 127 | 3 | 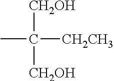 | H | H | CH₃(CH₂)₇CH═CH(CH₂)₇COOH | 1:1 | ⊚ | ◯ | ◯ | 0.116 | 0.129 | 0.131 |
| Working | 6 | (CH₂)₂OH | (CH₂)₂OH | (CH₂)₂OH | CH₃(CH₂)₇CH═CH(CH₂)₇COOH | 1:1 | ⊚ | ◯ | ◯ | 0.120 | 0.133 | 0.135 |
| example | | | | | | | | | | | | |
| 128 | | | | | | | | | | | | |
| Comparative | 27 | E-451D (Polyether-based non-ionic surfactant) | | | | | X | X | X | 0.130 | 0.142 | 0.153 |
| example | | | | | | | | | | | | |
| 54 | | | | | | | | | | | | |

TABLE 22A

| | Additive | | | | | | Emulsion composition | | | | Emulsion composition | | | | Emulsion composition | | | |
|---|---|---|---|---|---|---|---|---|---|---|---|---|---|---|---|---|---|---|
| | | | | | | Com- | Composition ratio (1) | | | | Composition ratio (2) | | | | Composition ratio (3) | | | |
| | | | | | | pound- | (wt. %) | | | | (wt. %) | | | | (wt. %) | | | |
| | | | | | | ing | | | Oil | | | | Oil | | | | Oil | |
| | | Component (A): | | | | molar | | | agent | Sur- | | | agent | Sur- | | | agent | Sur- |
| | | 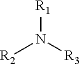 | | | | ratio | Addi- | Wa- | OTMP- | factant | Addi- | Wa- | OTMP | factant | Addi- | Wa- | OTMP- | factant |
| | No. | R₁ | R₂ | R₃ | Component (B) | (A):(B) | tive | ter | 300 | E-451D | tive | ter | 300 | E-451D | tive | ter | 300 | E-451D |
| Working example 129 | 8 | 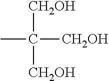 | H | H | CH₃(CH₂)₈CH((CH₂)₆CH₃₎COOH | 1:1 | 8 | 30 | 60 | 2 | 8 | 50 | 40 | 2 | 8 | 70 | 20 | 2 |
| Working example 130 | 9 | 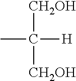 | H | H | CH₃(CH₂)₈CH((CH₂)₆CH₃₎COOH | 1:1 | 8 | 30 | 60 | 2 | 8 | 50 | 40 | 2 | 8 | 70 | 20 | 2 |
| Working example 131 | 10 | 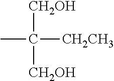 | H | H | CH₃(CH₂)₈CH((CH₂)₆CH₃₎COOH | 1:1 | 8 | 30 | 60 | 2 | 8 | 50 | 40 | 2 | 8 | 70 | 20 | 2 |
| Working | 13 | (CH₂)₂OH | (CH₂)₂OH | (CH₂)₂OH | CH₃(CH₂)₈CH((CH₂)₆CH₃₎COOH | 1:1 | 8 | 30 | 60 | 2 | 8 | 50 | 40 | 2 | 8 | 70 | 20 | 2 |
| example | | | | | | | | | | | | | | | | | | |
| 132 | | | | | | | | | | | | | | | | | | |

| | | | | | | | | | | | | | | |
|---|---|---|---|---|---|---|---|---|---|---|---|---|---|---|
| Compar- | 27 | E-451D (Polyether-based non-ionic surfactant | 8 | 30 | 60 | 2 | 8 | 50 | 40 | 2 | 8 | 70 | 20 | 2 |
| ative | | | | | | | | | | | | | | |
| example | | | | | | | | | | | | | | |
| 54 | | | | | | | | | | | | | | |

TABLE 22B

| | | | | | | | | | | Dynamic | | |
|---|---|---|---|---|---|---|---|---|---|---|---|---|
| | | | | | | | Stability (at 25° C.) | | | friction coefficient | | |
| | | | | | | | Emul- | Emul- | Emul- | Emul- | Emul- | Emul- |
| | | | | | | | sion | sion | sion | sion | sion | sion |
| | | | | | | | com- | com- | com- | com- | com- | com- |
| | Additive | | | | | | posi- | posi- | posi- | posi- | posi- | posi- |
| | | | | | | Com- | tion | tion | tion | tion | tion | tion |
| | | | | | | pounding | Compo- | Compo- | Compo- | Compo- | Compo- | Compo- |
| | | Component (A): | | | | molar | sition | sition | sition | sition | sition | sition |
| | | 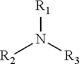 | | | | ratio | ratio (1) | ratio (2) | ratio (3) | ratio (1) | ratio (2) | ratio (3) |
| | No. | R₁ | R₂ | R₃ | Component (B) | (A):(B) | (wt. %) | (wt. %) | (wt. %) | (wt. %) | (wt.%) | (wt. %) |
| Work- ing exam- ple 129 | 8 | 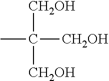 | H | H | CH₃(CH₂)₈CH ((CH₂)₆CH₃₎COOH | 1:1 | ⊚ | ⊚ | ⊚ | 0.116 | 0.121 | 0.134 |
| Work- ing exam- ple 130 | 9 | 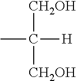 | H | H | CH₃(CH₂)₈CH ((CH₂)₆CH₃₎COOH | 1:1 | ⊚ | ◯ | ◯ | 0.119 | 0.126 | 0.136 |
| Work- ing exam- ple 131 | 10 | 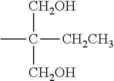 | H | H | CH₃(CH₂)₈CH ((CH₂)₆CH₃₎COOH | 1:1 | ⊚ | ◯ | ◯ | 0.121 | 0.133 | 0.136 |
| Work- | 13 | (CH₂)₂OH | (CH₂)₂OH | (CH₂)₂OH | CH₃(CH₂)₈CH | 1:1 | ⊚ | ◯ | ◯ | 0.127 | 0.135 | 0.137 |
| ing | | | | | ((CH₂)₆CH₃₎COOH | | | | | | | |
| exam- | | | | | | | | | | | | |
| ple | | | | | | | | | | | | |
| 132 | | | | | | | | | | | | |

| | | | | | | | | |
|---|---|---|---|---|---|---|---|---|
| Com- | 27 | E-451D (Polyether-based non-ionic surfactant) | x | x | x | 0.130 | 0.142 | 0.153 |
| par- | | | | | | | | |
| ative | | | | | | | | |
| exam- | | | | | | | | |
| ple | | | | | | | | |
| 54 | | | | | | | | |

### 11. Evaluation 3 of Composition Using Additive for Imparting Interfacial Activity

Composition ratios of the components (A) and (B) in the compositions that were employed in in Tables 19A, 19B to 22A and 22B were studied in Table 23. The results shows that the working examples 133 and 134 having an additive composition ratio of 5 wt. % which is less than those of the working examples 117 and 121 archived emulsion compositions, which in turn suggests that the additive of the present invention exhibits an enhanced interfacial activity even in the system of Table 23.

TABLE 23

| | | | | | | | | | | | | | Emulsion stability | |
|---|---|---|---|---|---|---|---|---|---|---|---|---|---|---|
| | | | | | | | | | | | | | | Evalua- |
| | Additive | | | | | | Emulsion composition | | | | | | | tion 2 |
| | | | | | | Com- | Composition ratio (wt. %) | | | | Emulsified | | Evalua- | Left to |
| | | | | | | pounding | | | Oil | | state | | tion 1 | stand |
| | | Component (A): | | | | molar | | | agent | Surfac- | Ap- | | Cen- | still at |
| | | 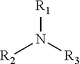 | | | | ratio | Addi- | | OTMP- | tant | pear- | Flu- | trifu- | 50° C. |
| | No. | R₁ | R₂ | R₃ | Component (B) | (A):(B) | tive | Water | 300 | E-451D | ance | idity | gation | for 14 days |
| Work- ing exam- ple 133 | 1 | 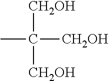 | H | H | CH₃(CH₂)₇ CH═CH (CH₂)₇COOH | 1:1 | 5 | 30 | 65 | 0 | ◯ | ◯ | ⊚ | ◯ |
| Work- ing exam- ple 134 | 8 | 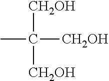 | H | H | CH₃(CH₂)₈CH ((CH₂)₆CH₃₎COOH | 1:1 | 5 | 30 | 65 | 0 | ◯ | ◯ | ⊚ | ◯ |

### 12. Metal Corrosion Evaluation of Additive

Corrosiveness was evaluated based on a testing method as set forth in JIS K2241 (cutting oil agent). SPCC steel plate (10 mm×10 mm×1 mm) as set forth in JIS G 3141, C1100P copper plate (10 mm×10 mm×1 mm) as set forth in JIS H 3100, A1050P aluminum plate (10 mm×10 mm×1 mm) as set forth in JIS H 4000 are respectively placed into 10 mL test tubes, and 3 g of the compositions formulated with the additives 1 to 3 and 8 to 10 (the working examples 135 to 140) prepared in the same manner as in section 9-1 were added to the respective test tubes in which the metal pieces were immersed, and they were left for 48 hours at room temperature (25° C.). The metal strips were then washed by the method as set forth in JIS to evaluate the presence or absence of discoloration on the metal pieces by visually comparing them with the corresponding untreated metal strips. As can be seen from the results in Tables 24 and 25, it was confirmed that the working examples 135 to 140 caused no discoloration on all of the metal strips of steel, copper, and aluminum, which shows that they exhibited no metal corrosiveness.

### 13. Thermal Stability Evaluation of Additives

The tests were carried out with reference to a testing method as set forth in “Petroleum products-Lubricating oils-Determination of thermal stability (JISK 2540)”. To 30 mL test tubes, there were respectively added 20 g of the samples which were same as in the section 9-1 (working examples 135 to 140), which were then left to stand still in a thermostatic bath of 170° C. for 12 hours. Then, the presence or absence of precipitate was visually confirmed to evaluate their thermal stabilities (Tables 24 and 25).

From the results in Tables 24 and 25, no precipitations were found for all of the working examples 130 to 140 after the heat application, and it was found that they were excellent in thermal stability. That is, as the additive or composition of the present invention is excellent in thermal stability and corrosiveness, it can be inferred that the additive or composition of the present invention is greatly of practical use in a variety of fields such as lubricants.

TABLE 24

| | Additive | | | | | | | | | | | | | |
|---|---|---|---|---|---|---|---|---|---|---|---|---|---|---|
| | | | | | | Com- | Emulsion composition | | | | Metal corrosiveness | | | Thermal |
| | | | | | | pound- | Composition ratio (1) (wt. %) | | | | Emulsion | | | stability |
| | | | | | | ing | | | Oil | Surfac- | composition | | | Emulsion |
| | | Component (A): | | | | molar | | | agent | tant | ratio \textcircled{1} | | | composition |
| | | 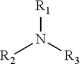 | | | Component | ratio | Addi- | | OTMP- | E- | | | Alu- | Composition |
| | No. | R₁ | R₂ | R₃ | (B) | (A):(B) | tive | Water | 300 | 451D | Steel | Copper | minium | ratio (1) |
| Work- ing exam- ple 135 | 1 | 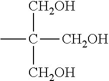 | H | H | CH₃(CH₂)₇ CH═CH (CH₂)₇COOH | 1:1 | 10 | 30 | 60 | 0 | No dis- color- ation was ob- served | No discolor- ation was observed | No discolor- ation was observed | No precipitate was observed |
| Work- ing exam- ple 136 | 2 | 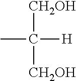 | H | H | CH₃(CH₂)₇ CH═CH (CH₂)₇COOH | 1:1 | 10 | 30 | 60 | 0 | No dis- color- ation was ob- served | No discolor- ation was observed | No discolor- ation was observed | No precipitate was observed |
| Work- ing exam- ple 137 | 3 | 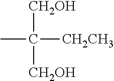 | H | H | CH₃(CH₂)₇ CH═CH (CH₂)₇COOH | 1:1 | 10 | 30 | 60 | 0 | No dis- color- ation was ob- served | No discolor- ation was observed | observed No discolor- ation was | No precipitate was observed |

TABLE 25

| | | | | | | | | | | | | | | Thermal |
|---|---|---|---|---|---|---|---|---|---|---|---|---|---|---|
| | Additive | | | | | | Emulsion composition | | | | | | | stability |
| | | | | | | Com- | Composition | | | | Metal corrosiveness | | | Emulsion |
| | | | | | | pound- | ratio (1) (wt. %) | | | | Emulsion | | | compo- |
| | | | | | | ing | | | Oil | Surfac- | composition | | | sition |
| | | Component (A): | | | | molar | | | agent | tant | ratio \textcircled{1} | | | Compo- |
| | | 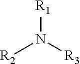 | | | | ratio | Addi- | Wa- | OTMP- | E- | | | Alu- | sition |
| | No. | R₁ | R₂ | R₃ | Component (B) | (A):(B) | tive | ter | 300 | 451D | Steel | Copper | minium | ratio (1) |
| Work- ing exam- ple 138 | 8 | 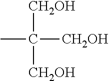 | H | H | CH₃(CH₂)₈CH ((CH₂)₆CH₃₎COOH | 1:1 | 10 | 30 | 60 | 0 | No dis- color- ation was ob- served | No dis- color- ation was ob- served | No dis- color- ation was ob- served | No precip- itate was observed |
| Work- ing exam- ple 139 | 9 | 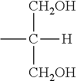 | H | H | CH₃(CH₂)₈CH ((CH₂)₆CH₃₎COOH | 1:1 | 10 | 30 | 60 | 0 | No dis- color- ation was ob- served | No dis- color- ation ob- served | No dis- color- ation was ob- served | No precip- itate was observed |
| Work- ing exam- ple 140 | 10 | 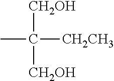 | H | H | CH₃(CH₂)₈CH ((CH₂)₆CH₃₎COOH | 1:1 | 10 | 30 | 60 | 0 | No disc- olor- ation was ob- served | No dis- color- ation was ob- ob- served | No dis- color- ation was ob- served | No precip- itate was observed |

### 14. Composition Evaluation and Lubricity Evaluation 2 of Composition in Parallel Use of Surfactant and Additive for Imparting Interfacial Activity.

### 14-1. Composition Evaluation

The compositions of the working examples 149 to 154 and the comparative examples 58 to 61 were prepared. The components (A) and (B) of the present invention and a solvent were mixed with each other at the composition ratios (1) to (3) as listed in Tables 26A, 26B, 26C, 27A, 27B, 27C, 28A, 28B and 28C, and the additives, having the solvent distilled away, water, an oil agent and a surfactant were mixed and stirred for three hours at room temperature to obtain an emulsion composition. A similar emulsion composition was obtained by mixing the components (A) and (B) as the additives of the present invention in a separate manner into an oil agent and water such that the composition ratios thereof were as listed in Tables 26A, 26B, 26C, 27A, 27B, 27C, 28A, 28B and 28C, and stirring them at room temperature for three hours.

The resultant emulsion compositions had low electric conductivities as a result of electric conductance measurement, and the continuous phase was of an oil phase, which lead to the conclusion that the resultant emulsion compositions were of W/O type emulsion.

Stabilities of the resultant emulsion compositions were evaluated on the basis of the same criteria of the section 9-1. It was found from the results in Tables 26A, 26B, 26C, 27A, 27B, 27C, 28A, 28B and 28C that the working examples 149 to 154 of the present invention resulted in emulsion compositions with reference to those of the comparative examples 58 to 61.

Further, as for the correlation between the structure of the components (A) and (B) and the interfacial activity (emulsification) effects, a tendency similar to the correlation between the structure of the components (A) and (B) and the interfacial activity (emulsification) effects as set forth in the section 9 was observed.

### 14-2. Lubricity Evaluation

Dynamic friction coefficients of the compositions of the working examples 149 to 154 and the comparative examples 58 to 61 as prepared for the composition evaluation mentioned above were measured using pendulum type oiliness friction tester (KOBELCO MACHINERY ENGINEERING Co., Ltd.).

As shown from the results in Tables 26A, 26B, 26C, 27A, 27B, 27C, 28A, 28B and 28C, the respective emulsion compositions (at the composition ratios (1) to (3)) of the working examples 149 to 154 had smaller dynamic friction coefficients compared to those of the comparative examples 58 to 61, which therefore suggests that the additives containing the components (A) and (B) according to the present invention contributed to the formations of the highly stable emulsion compositions, which I turn caused the emulsion compositions to have an enhanced affinity to the friction target material to thereby exhibit an enhanced lubrication effect. Further, as for the correlation between the structure of the components (A) and (B) and the dynamic friction coefficient, a tendency similar to the correlation between the structure of the components (A) and (B) and the interfacial activity (emulsification) effects as set forth in the section 9-2 was observed.

TABLE 26A

| | | | | | | | Emulsion composition | | | |
|---|---|---|---|---|---|---|---|---|---|---|
| | | | | | | | Composition ratio (1) (wt %) | | | |
| | Additive | | | | | | | | | Surfac- |
| | | | | | | Com- | | | Oil | tant |
| | | Component (A): | | | | pounding | | | agent | Glycerin |
| | | 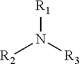 | | | | molar ratio | | | OTMP- | fatty acid |
| | No. | R₁ | R₂ | R₃ | Component (B) | (A): (B) | Additive | Water | 300 | ester |
| Working example 149 | 1 | 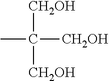 | H | H | CH₃(CH₂)₇ CH═CH(CH₂)₇COOH | 1:1 | 8 | 30 | 60 | 2 |
| Working | 6 | (CH₂)₂OH | (CH₂)₂OH | (CH₂)₂OH | CH₃(CH₂)₇ | 1:1 | 8 | 30 | 60 | 2 |
| example | | | | | CH═CH(CH₂)₇COOH | | | | | |
| 150 | | | | | | | | | | |
| Compar- | 27 | E-451D (Polyether-based non-ionic surfactant) | | | | | 8 | 30 | 60 | 2 |
| ative | | | | | | | | | | |
| example | | | | | | | | | | |
| 58 | | | | | | | | | | |
| Compar- | 30 | Glycerin fatty acid ester | | | | | 8 | 30 | 60 | 2 |
| ative | | | | | | | | | | |
| example | | | | | | | | | | |
| 59 | | | | | | | | | | |

| | Emulsion composition | | | | Emulsion composition | | | |
|---|---|---|---|---|---|---|---|---|
| | Composition ratio (2) (wt %) | | | | Composition ratio (3) (wt %) | | | |
| | | | | Surfactant | | | | Surfactant |
| | | | | Glycerin | | | | Glycerin |
| | | | Oil agent | fatty acid | | | Oil agent | fatty acid |
| | Additive | Water | OTMP-300 | ester | Additive | Water | OTMP-300 | ester |
| Working | 8 | 50 | 40 | 2 | 8 | 70 | 20 | 2 |
| example 149 | | | | | | | | |
| Working | 8 | 50 | 40 | 2 | 8 | 70 | 20 | 2 |
| example 150 | | | | | | | | |
| Comparative | 8 | 50 | 40 | 2 | 8 | 70 | 20 | 2 |
| example 58 | | | | | | | | |
| Comparative | 8 | 50 | 40 | 2 | 8 | 70 | 20 | 2 |
| example 59 | | | | | | | | |

TABLE 26C

| | | | | | | | Stability (at 25° C.) | | | Dynamic friction coefficient | | |
|---|---|---|---|---|---|---|---|---|---|---|---|---|
| | | | | | | | Emul- | Emul- | Emul- | Emul- | Emul- | Emul- |
| | Additive | | | | | | sion | sion | sion | sion | sion | sion |
| | | | | | | Com- | compo- | compo- | compo- | compo- | compo- | compo- |
| | | | | | | pounding | sition | sition | sition | sition | sition | sition |
| | | Component (A): | | | | molar | Compo- | Compo- | Compo- | Compo- | Compo- | Compo- |
| | | 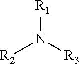 | | | Component | ratio | sition | sition | sition | sition | sition | sition |
| | No. | R₁ | R₂ | R₃ | (B) | (A):(B) | ratio (1) | ratio (2) | ratio (3) | ratio (1) | ratio (2): | ratio (3) |
| Work- ing exam- ple 149 | 1 | 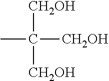 | H | H | CH₃(CH₂)₇ CH═CH (CH₂)₇COOH | 1:1 | ⊚ | ⊚ | ⊚ | 0.113 | 0.120 | 0.123 |
| Work- | 6 | (CH₂)₂OH | (CH₂)₂OH | (CH₂)₂OH | CH₃(CH₂)₇ | 1:1 | ⊚ | ◯ | ◯ | 0.123 | 0.126 | 0.128 |
| ing | | | | | CH═CH | | | | | | | |
| exam- | | | | | (CH₂)₇COOH | | | | | | | |
| ple | | | | | | | | | | | | |
| 150 | | | | | | | | | | | | |
| Com- | 27 | E-451D (Polyether-based non-ionic surfactant) | | | | | x | x | x | 0.135 | 0.148 | 0.155 |
| par- | | | | | | | | | | | | |
| ative | | | | | | | | | | | | |
| exam- | | | | | | | | | | | | |
| ple | | | | | | | | | | | | |
| 58 | | | | | | | | | | | | |
| Com- | 30 | Glycerin fatty acid ester | | | | | x | x | x | 0.135 | 0.145 | 0.158 |
| par- | | | | | | | | | | | | |
| ative | | | | | | | | | | | | |
| exam- | | | | | | | | | | | | |
| ple | | | | | | | | | | | | |
| 59 | | | | | | | | | | | | |

TABLE 26D

| | | | | | | | Emulsion composition | | | |
|---|---|---|---|---|---|---|---|---|---|---|
| | | | | | | | Composition ratio (1) (wt %) | | | |
| | Additive | | | | | | | | | Surfac- |
| | | | | | | Com- | | | Oil | tant |
| | | Component (A): | | | | pounding | | | agent | Glycerin |
| | | 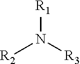 | | | | molar ratio | | | OTMP- | fatty acid |
| | No. | R₁ | R₂ | R₃ | Component (B) | (A):(B) | Additive | Water | 300 | ester |
| Working example 151 | 8 | 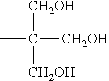 | H | H | CH₃(CH₂)₈CH((CH₂)₆CH₃₎COOH | 1:1 | 8 | 30 | 60 | 2 |
| Working | 13 | (CH₂)₂OH | (CH₂)₂OH | (CH₂)₂OH | CH₃(CH₂)₈CH((CH₂)₆CH₃₎COOH | 1:1 | 8 | 30 | 60 | 2 |
| example | | | | | | | | | | |
| 152 | | | | | | | | | | |
| Compar- | 27 | E-451D (Polyether-based non-ionic surfactant) | | | | | 8 | 30 | 60 | 2 |
| ative | | | | | | | | | | |
| example | | | | | | | | | | |
| 58 | | | | | | | | | | |
| Compar- | 30 | Glycerin fatty acid ester | | | | | 8 | 30 | 60 | 2 |
| ative | | | | | | | | | | |
| example | | | | | | | | | | |
| 59 | | | | | | | | | | |

| | Emulsion composition | | | | Emulsion composition | | | |
|---|---|---|---|---|---|---|---|---|
| | Composition ratio (2) (wt %) | | | | Composition ratio (3) (wt %) | | | |
| | | | | Surfactant | | | | Surfactant |
| | | | | Glycerin | | | | Glycerin |
| | | | Oil agent | fatty acid | | | Oil agent | fatty acid |
| | Additive | Water | OTMP-300 | ester | Additive | Water | OTMP-300 | ester |
| Working | 8 | 50 | 40 | 2 | 8 | 70 | 20 | 2 |
| example 151 | | | | | | | | |
| Working | 8 | 50 | 40 | 2 | 8 | 70 | 20 | 2 |
| example 152 | | | | | | | | |
| Comparative | 8 | 50 | 40 | 2 | 8 | 70 | 20 | 2 |
| example 58 | | | | | | | | |
| Comparative | 8 | 50 | 40 | 2 | 8 | 70 | 20 | 2 |
| example 59 | | | | | | | | |

TABLE 27C

| | | | | | | | Stability (at 25° C.) | | | Dynamic friction coefficient | | |
|---|---|---|---|---|---|---|---|---|---|---|---|---|
| | | | | | | | Emul- | Emul- | Emul- | Emul- | Emul- | Emul- |
| | Additive | | | | | | sion | sion | sion | sion | sion | sion |
| | | | | | | Com- | compo- | compo- | compo- | compo- | compo- | compo- |
| | | | | | | pounding | sition | sition | sition | sition | sition | sition |
| | | Component (A): | | | | molar | Compo- | Compo- | Compo- | Compo- | Compo- | Compo- |
| | | 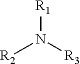 | | | | ratio | sition | sition | sition | sition | sition | sition |
| | No. | R₁ | R₂ | R₃ | Component (B) | (A):(B) | ratio (1) | ratio (2) | ratio (3) | ratio (1) | ratio (2) | ratio (3) |
| Working example 151 | 8 | 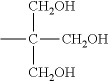 | H | H | CH₃(CH₂)₈CH ((CH₂)₆CH₃₎COOH | 1:1 | ⊚ | ⊚ | ⊚ | 0.123 | 0.129 | 0.135 |
| Working | 13 | (CH₂)₂OH | (CH₂)₂OH | (CH₂)₂OH | CH₃(CH₂)₈CH | 1:1 | ⊚ | ◯ | ◯ | 0.124 | 0.127 | 0.129 |
| example | | | | | ((CH₂)₆CH₃₎COOH | | | | | | | |
| 152 | | | | | | | | | | | | |
| Compar- | 27 | E-451D (Polyether-based non-ionic surfactant) | | | | | x | x | x | 0.135 | 0.148 | 0.155 |
| ative | | | | | | | | | | | | |
| example | | | | | | | | | | | | |
| 58 | | | | | | | | | | | | |
| Compar- | 30 | Glycerin fatty acid ester | | | | | x | x | x | 0.135 | 0.145 | 0.158 |
| ative | | | | | | | | | | | | |
| example | | | | | | | | | | | | |
| 59 | | | | | | | | | | | | |

TABLE 28A

| | | | | | | | Emulsion composition | | | |
|---|---|---|---|---|---|---|---|---|---|---|
| | | | | | | | Composition ratio (1) (wt %) | | | |
| | | | | | | | | | | Surfac- |
| | Additive | | | | | | | | | tant |
| | | | | | | Com- | | | Oil | Glycerin |
| | | Component (A): | | | | pounding | | | agent | fatty |
| | | 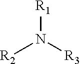 | | | | molar ratio | Addi- | Wa- | OTMP- | acid |
| | No. | R₁ | R₂ | R₃ | Component (B) | (A):(B) | tive | ter | 300 | ester |
| Working example 153 | 1 | 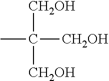 | H | H | CH₃(CH₂)₇ CH═CH(CH₂)₇COOH | 1:1 | 4 | 30 | 60 | 6 |
| Working | 6 | (CH₂)₂OH | (CH₂)₂OH | (CH₂)₂OH | CH₃(CH₂)₇ | 1:1 | 4 | 30 | 60 | 6 |
| example | | | | | CH═CH(CH₂)₇COOH | | | | | |
| 154 | | | | | | | | | | |
| Compar- | 27 | E-451D (Polyether-based non-ionic surfactant) | | | | | 4 | 30 | 60 | 6 |
| ative | | | | | | | | | | |
| example | | | | | | | | | | |
| 60 | | | | | | | | | | |
| Compar- | 30 | Glycerin fatty acid ester | | | | | 4 | 30 | 60 | 6 |
| ative | | | | | | | | | | |
| example | | | | | | | | | | |
| 61 | | | | | | | | | | |

| | Emulsion composition | | | | Emulsion composition | | | |
|---|---|---|---|---|---|---|---|---|
| | Composition ratio (2) (wt %) | | | | Composition ratio (3) (wt %) | | | |
| | | | | Surfactant | | | | Surfactant |
| | | | | Glycerin | | | | Glycerin |
| | | | Oil agent | fatty acid | | | Oil agent | fatty acid |
| | Additive | Water | OTMP-300 | ester | Additive | Water | OTMP-300 | ester |
| Working | 4 | 50 | 40 | 6 | 4 | 70 | 20 | 6 |
| example 153 | | | | | | | | |
| Working | 4 | 50 | 40 | 6 | 4 | 70 | 20 | 6 |
| example 154 | | | | | | | | |
| Comparative | 4 | 50 | 40 | 6 | 4 | 70 | 20 | 6 |
| example 60 | | | | | | | | |
| Comparative | 4 | 50 | 40 | 6 | 4 | 70 | 20 | 6 |
| example 61 | | | | | | | | |

TABLE 28C

| | | | | | | | Stability (at 25° C.) | | | Dynamic friction coefficient | | |
|---|---|---|---|---|---|---|---|---|---|---|---|---|
| | | | | | | | Emul- | Emul- | Emul- | Emul- | Emul- | Emul- |
| | Additive | | | | | | sion | sion | sion | sion | sion | sion |
| | | | | | | Com- | compo- | compo- | compo- | compo- | compo- | compo- |
| | | | | | | pounding | sition | sition | sition | sition | sition | sition |
| | | Component (A): | | | | molar | Compo- | Compo- | Compo- | Compo- | Compo- | Compo- |
| | | 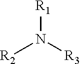 | | | Component | ratio | sition | sition | sition | sition | sition | sition |
| | No. | R₁ | R₂ | R₃ | (B) | (A):(B) | ratio (1) | ratio (2) | ratio (3) | ratio (1) | ratio (2) | ratio (3) |
| Work- ing exam- ple 153 | 1 | 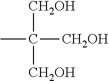 | H | H | CH₃(CH₂)₇ CH═CH (CH₂)₇COOH | 1:1 | ⊚ | ⊚ | ⊚ | 0.112 | 0.115 | 0.116 |
| Work- | 6 | (CH₂)₂OH | (CH₂)₂OH | (CH₂)₂OH | CH₃(CH₂)₇ | 1:1 | ⊚ | ◯ | ◯ | 0.124 | 0.127 | 0.129 |
| ing | | | | | CH═CH | | | | | | | |
| exam- | | | | | (CH₂)₇COOH | | | | | | | |
| ple | | | | | | | | | | | | |
| 154 | | | | | | | | | | | | |
| Com- | 27 | E-451D (Polyether-based non-ionic surfactant) | | | | | x | x | x | 0.135 | 0.148 | 0.155 |
| par- | | | | | | | | | | | | |
| ative | | | | | | | | | | | | |
| exam- | | | | | | | | | | | | |
| ple | | | | | | | | | | | | |
| 60 | | | | | | | | | | | | |
| Com- | 30 | Glycerin fatty acid ester | | | | | x | x | x | 0.135 | 0.145 | 0.158 |
| par- | | | | | | | | | | | | |
| ative | | | | | | | | | | | | |
| exam- | | | | | | | | | | | | |
| ple | | | | | | | | | | | | |
| 61 | | | | | | | | | | | | |

1. An additive for surface activation, wherein the additive is a formulation comprising the following components:
(A) an amine or ammonium compound having a hydroxyaliphatic hydrocarbon group that contains one or more hydroxy groups and optionally contains an oxygen atom unassociated with the hydroxy groups; and
(B) an unsaturated or branched aliphatic acid having 8 to 22 carbon atoms or a salt thereof.

2. The additive for surface activation according to claim 1, wherein the component (A) is an amine or ammonium compound comprising a branched hydroxyaliphatic hydrocarbon group having one or more hydroxy groups and optionally containing an oxygen atom unassociated with the hydroxy groups.

3. The additive for surface activation according to claim 1,
wherein the component (A) is represented by the formula (I)
[Chemical formula 1]
N[R¹]ₘ[R²]₃₋ₘ (I)
wherein R¹ independently represents a hydroxyaliphatic hydrocarbon group having one or more hydroxy groups in which a hydrocarbon moiety thereof has 1 to 22 carbon atoms, said hydrocarbon moiety optionally containing an oxygen atom, R² independently represents a hydrogen atom or an organic group having 1 to 22 carbon atoms, and m represents an integer of 1 to 3; or
wherein the component (A) is represented by the formula (II)
[Chemical formula 2]
N⁺[R³]ₙ[R⁴]₄₋ₙX⁻ (II)
wherein R³ independently represents a hydroxyaliphatic hydrocarbon group having one or more hydroxy groups in which a hydrocarbon moiety thereof has 1 to 22 carbon atoms, said hydrocarbon moiety optionally containing an oxygen atom, R⁴ independently represents a hydrogen atom or an organic group having 1 to 22 carbon atoms, n represents an integer of 1 to 4, and X represents an anion.

4. The additive for surface activation according to claim 3, wherein all of the R²s in the formula (I) are hydrogen atoms, and all of the R⁴s in the formula (II) are hydrogen atoms.

5. The additive for surface activation according to claim 3, wherein the hydroxyaliphatic hydrocarbon group in the formula (I) or (II) is a saturated hydroxyaliphatic hydrocarbon group whose hydrocarbon moiety has 1 to 22 carbon atoms, said hydrocarbon moiety optionally containing an oxygen atom.

6. The additive for surface activation according to claim 3,
wherein at least one of the R¹s in the formula (I) is a saturated hydroxyaliphatic hydrocarbon group having 1 to 3 hydroxy groups in which a hydrocarbon moiety thereof has 1 to 22 carbon atoms, said hydrocarbon moiety optionally containing an oxygen atom, and wherein at least one of the R³s in the formula (II) is a saturated hydroxyaliphatic hydrocarbon group having 1 to 3 hydroxy groups in which a hydrocarbon moiety thereof has 1 to 22 carbon atoms, said hydrocarbon moiety optionally containing an oxygen atom.

7. The additive for surface activation according to claim 3,
wherein at least one of the R¹s in the formula (I) is a linear saturated hydroxyaliphatic hydrocarbon group having one hydroxy group in which a hydrocarbon moiety thereof has 1 to 22 carbon atoms, and
wherein at least one of the R³s in the formula (II) is a linear saturated hydroxyaliphatic hydrocarbon group having one hydroxy group in which a hydrocarbon moiety thereof has 1 to 22 carbon atoms.

8. The additive for surface activation according to claim 3,
wherein at least one of the R¹s in the formula (I) is a branched hydroxyaliphatic hydrocarbon group having one or more hydroxy groups in which a hydrocarbon moiety thereof has 3 to 12 carbon atoms, and
wherein at least one of the R³s in the formula (II) is a branched hydroxyaliphatic hydrocarbon group having one or more hydroxy groups in which a hydrocarbon moiety thereof has 3 to 12 carbon atoms.

9. The additive for surface activation according to claim 3, wherein X⁻ in the formula (II) is a hydroxide ion.

10. The additive for surface activation according to claim 3, wherein the component (A) is represented by the formula (I).

11. The additive for surface activation according to claim 1, wherein the additive comprises an organic salt formed by: a cation originated from the component (A); and an anion originated from an anionic residue of the component (B), wherein said cation optionally contains a cationic residue of the component (B).

12. The additive for surface activation according to claim 11, wherein the cation is an ammonium cation.

13. The additive for surface activation according to claim 11, wherein the cation of the organic ammonium salt formed by the components (A) and (B) is represented by the formula (II):
[Chemical formula 3]
N⁺[R⁵]ₒ[R⁶]₄₋ₒ (III)
wherein R⁵ independently represents a hydroxyaliphatic hydrocarbon group having one or more hydroxy groups in which a hydrocarbon moiety thereof has 1 to 22 carbon atoms, said hydrocarbon moiety optionally containing an oxygen atom, R⁶ independently represents a hydrogen atom or an organic group having 1 to 22 carbon atoms, and o represents an integer of 1 to 4.

14. The additive for surface activation according to claim 13, wherein all of the R¹s in the formula (III) are hydrogen atoms.

15. The additive for surface activation according to claim 13, wherein the hydroxyaliphatic hydrocarbon group in the formula (III) is a saturated hydroxyaliphatic hydrocarbon group whose hydrocarbon moiety has 1 to 22 carbon atoms, said hydrocarbon moiety optionally containing an oxygen atom.

16. The additive for surface activation according to claim 13, wherein at least one of the R¹s in the formula (I) is a saturated hydroxyaliphatic hydrocarbon group having 1 to 3 hydroxy groups in which a hydrocarbon moiety thereof has 1 to 22 carbon atoms, said hydrocarbon moiety optionally containing an oxygen atom.

17. The additive for surface activation according to claim 13, wherein at least one of the R¹s in the formula (III) is a linear saturated hydroxyaliphatic hydrocarbon group having one hydroxy group in which a hydrocarbon moiety thereof has 1 to 22 carbon atoms.

18. The additive for surface activation according to claim 13, wherein at least one of the R¹s in the formula (III) is a branched hydroxyaliphatic hydrocarbon group having one or more hydroxy groups in which a hydrocarbon moiety thereof has 3 to 12 carbon atoms.

19. The additive for surface activation according to claim 13, wherein when at least one of the R⁶s in the cation expressed by the formula (III) is a hydrogen atom, a proton corresponding to said hydrogen atom is originated from the component (B).

20. The additive for surface activation according to claim 1, wherein the component (B) is an unsaturated aliphatic acid having 8 to 22 carbon atoms or a salt thereof.

21. The additive for surface activation according to claim 1, wherein the component (B) is a branched saturated aliphatic acid having 8 to 22 carbon atoms or a salt thereof.

22. The additive for surface activation according to claim 1, wherein an anhydride and/or hydrate of a salt of the components (A) and (B) or a mixture of the components (A) and (B) is liquid at 25° C.

23. An emulsion composition comprising the additive according to claim 1.

24. A cosmetic as an emulsion composition comprising the additive according to claim 1.

25. A lubricant as an emulsion composition comprising the additive according to claim 1.
